SEQUENCE LISTING (68 sequences)

SEQ ID NO: 1 - DNA, 24 bp, Artificial Sequence
  Oligonucleotide primer for amplification of R1877 EcoRI marker sequence.
cattcctgcttccatggaaacgtc

SEQ ID NO: 2 - DNA, 33 bp, Artificial Sequence
  Oligonucleotide primer for amplification of R1877 EcoRI marker sequence.
ctctttctgtatacttgagctttgacatctgac

SEQ ID NO: 3 - DNA, 20 bp, Artificial Sequence
  Oligonucleotide primer for amplification of G4003 HindIII marker sequence.
gatcgacgagtacctgaacg

SEQ ID NO: 4 - DNA, 24 bp, Artificial Sequence
  Oligonucleotide primer for amplification of G4003 HindIII marker sequence.
aatagttggattgtcctcaaaggg

SEQ ID NO: 5 - DNA, 27 bp, Artificial Sequence
  Oligonucleotide primer for amplification of C1361 MwoI marker sequence.
aaagcaaccgacttcagtggcatcacc

SEQ ID NO: 6 - DNA, 24 bp, Artificial Sequence
  Oligonucleotide primer for amplification of C1361 MwoI marker sequence.
ctggacttcatttccctgcagagc

SEQ ID NO: 7 - DNA, 27 bp, Artificial Sequence
  Oligonucleotide primer for amplification of G2155 MwoI marker sequence.
gaccaccaattaactgattaagctggc

SEQ ID NO: 8 - DNA, 27 bp, Artificial Sequence
  Oligonucleotide primer for amplification of G2155 MwoI marker sequence.
tttctggctccaataatcagctgtagc

SEQ ID NO: 9 - DNA, 27 bp, Artificial Sequence
  Oligonucleotide primer for amplification of G291 MspI marker sequence.
ctgctgcagcaagctgcaccgaaccgg

SEQ ID NO: 10 - DNA, 24 bp, Artificial Sequence
  Oligonucleotide primer for amplification of G291 MspI marker sequence.
acattttttcttccgaaacttccg

SEQ ID NO: 11 - DNA, 24 bp, Artificial Sequence
  Oligonucleotide primer for amplification of R2303 BslI marker sequence.
atggaaagatacactagaatgagc

SEQ ID NO: 12 - DNA, 24 bp, Artificial Sequence
  Oligonucleotide primer for amplification of R2303 BslI marker sequence.
atcttatatagtggcaggaaagcc

SEQ ID NO: 13 - DNA, 24 bp, Artificial Sequence
  Oligonucleotide primer for amplification of S10019 BstUI marker sequence.
aacaatcttatcctgcacagactg

SEQ ID NO: 14 - DNA, 24 bp, Artificial Sequence
  Oligonucleotide primer for amplification of S10019 BstUI marker sequence.
gtcacatagaagcagatgggttcc

SEQ ID NO: 15 - DNA, 24 bp, Artificial Sequence
  Oligonucleotide primer for amplification of S10602 KpnI marker sequence.
agctgttgagagttctatgccacc

SEQ ID NO: 16 - DNA, 24 bp, Artificial Sequence
  Oligonucleotide primer for amplification of S10602 KpnI marker sequence.
tagccatgcaacaagatgtcatac

SEQ ID NO: 17 - DNA, 26 bp, Artificial Sequence
  Oligonucleotide primer for amplification of S12564 Tsp509I marker sequence.
ctagttagaccgaataactgaggttc

SEQ ID NO: 18 - DNA, 27 bp, Artificial Sequence
  Oligonucleotide primer for amplification of S12564 Tsp509I marker sequence.
tttgtgggtttgtggcattgagaaaat

SEQ ID NO: 19 - DNA, 2240 bp, Oryza sativa L.
  PCR marker G4003 HindIII
gcggccgctccgggaagtcgagcgagtagacgcccctgacgccgtacgcgtcggcgagccgcagcggcgtctctggcggtgtgaaggacagcccgttcagcgtcgcgcggcgccgcccgttgatcgtcaccggcgccgtgctccgcagcaggtacgcctgcgtcacgttgatcgacgagtacctgaacgatccctgtgggttcggcctcgccgctccggcactcaggttccacctgcccaatgcaaaaaaccaaaacccaaaagcttaatgcgaataatacatcattccacgtatttaaaaaaataatttataggtaaaatttttataatgtattttagcgacgtaaatgtcaatgctgagaaataaacgataatactttaaatgaagttctaaaatttaaattttggcatcggttgatgttggataaagaaaacgatggaggctagtaatttttcttcttttttaagtatctagattgtcatatattgaatttttcagtttttcatccctttgaggacaatccaactattattttccttttcttatgtaaaaggttgaacaacatattcaaacataaaaaaataaaattaaatgaaataaatttacaattcataaaatttacagaatttatgttaagaaaatattcaaacttagataataataaagcaacaaaatcgtactaaaaagaagtataattgtacattgtatactactactcctacaattttagacttagaatttttaatttcctgaaatctagtaatgccatttttttctttctagttgaaccagacagtaagtttaactcgaaacttataagctaatgagcgaagtcgggcaattcactcgtacctgacggagcgagcttggttcatggagaaggacttgtcgaactggtcctggggagggtcggggagcgggccggaggcccgcccccgggagttggagtagcggaggacggcgacgccggcgacgcggcgccacacggtgtcgttcaccatgcgcgcgctggcgacgacgtagtagtcggagctcgcgttctggtcggtggtgacgaggaaggagtaggactggccgacgtggacgtccaggttggtgtagttctgctgcgtcgtgtaggagccctccgtctccaccagcaccatgttgtgcccctggatcctgaagttgaggctcgtcgacgtccccacgttgtgcactcggatcctgtacgtcttgcctgtgtccccacaccgacgtcgccgacacacgcgcaaaagataatagactcattgtaagtaggtagtaaccttctccgtttcatattataaatcgtttgattatatttttgttagttaaacttctttaagttttttttctataaacttaattaaatctaaagaattttaataaaaaaaatcaaacgacttataatataaaatggatggagtagttgcatcaatttgtggatgaagcaaacaagattatatccttttcatgagggtgaaagtattcagtgaacaattcgtcagtttcaagtttcatgaaatcggacagggtctctgaaagtctgtatttttggtactgttggattgactactctggcttctgttgtcacatcttttgtatcctagtttcggtaaaaaaaattttggcatttttactcctatcgttgatctgtttaactgaaaccattgcatgatatactactagcagacaaaactggtgaaaattcacgagaatgaactttttgtcagttaagcattagcggacagcttcagtaagcagagcaggctgccttaaggcttaaagcactatcttccacaacactttgtcctacaatcaaattccaaatttactatcacaaaaagcgaaggaactaactaaaccttactcctactagtactactgctatgactatgaaacaagattccaatccaaagaaaacacagtgctcgatcagcatgataaaagcaacgaaacctgctcatccagctgccaaaatgccaccccactgactctacgtacgtactacgtattgacgctgtaaaaaactagccgtagtacagagaagaggacccaaagtttcgtcaaaaattttattttacccggatccacattgatggtctcgtactcgatgccggccgggacaaggctgtcgttgtacctgtacgggcccttgccgttaatcagcacgccgtccggcatcccgaggtccttgccactgtccagcatcttcctcagatcctgcaacgaattc

SEQ ID NO: 20 - DNA, 2601 bp, Oryza sativa L.
  PCR marker C1361 MwoI
tcttgctgagatccaagttgcggtaactttgcccttttctttttttcttctcttctgaattttttcatggtttttgggagagattttcgtaacttgattacagttctaggaaaaggccaccttgttcaaacagggctttcttgaaagggatcaatttgctaggagtacatgattctaaaagcgatttcgaaataaaacacagttctcgatctcatacctgaaaacaaaaggcccatactgtgtaaactgtgattatgcttctgttaaatgggatatttgtacaaaattgacgccaaccacctataaacagattgtgagcttttatcttagtaaaataaaatgtgacattctactcagtgttcagtgatccgatgtcgtctcttctgcgtacaacttctaacagccgttttcggtagtacaaactagcgaaacaccaaaaacgcagcatttgagttctggaatacgctgaaattgttagaatcaaccacgaaaccaaaatcattgttcagaaacgttgcaacgagataaaacacaagaacttgttttaacaaagcatacggacagtacatatacggttacaacacccagtctttatacagttctgctggagttccatctactggctgtcattgtatctcaggacagacaggttaacataggtacaacacaattacaggctaaaccgaagcgaactacactgtcagcatctctaacagtatcgtcaagcaagcttatttacagctgctctagtaaatttacaacgtccctggcagaatccctctcgtttctggcagcgacgaggcacggtccatggccttagcaggacatctcacccgtcagctgcatagaaagcaaccgacttcagtggaatcacctcctgctcctgcaaaaaagttggttcgatcaatcacgcgtttaatccaaaacaaaatgggtattaattatgctagcctatgaagctacctcagagttctctatttgctctgcagggaaatgaagtccagtggaacagttctcaagcacctcagggctcttcatccatgctttgtgtgcttcaatggctttcagcttatagcgaaacatctgcgatacggatctaaaattaaggatgtcgacaattacttaacacaacaaataattgaagcaggtccagttaaagaaaagtagcagcgaagaatagcactctgaagtctgaacctcagataaagaaatggttggtttttccagttcatctccctcaacatggattccagtaccctggcattctgggcaaaggatggatgttattttcttaggtgcattttttgcctttcttcctcgattgctttttcccttgcttgcaattttgtctgctagcatctcatattggcataaaatagtccagtgcacaaggcaagaagtgtgaaacaaatgaaatgcctgcaaaattagccgtacaaagtcattggaggttgcagcagaatactacaaatttttaaagaagaaactatacactgtctatgttttgcttgaaatgaattcaaccactttgcattatacggtttggaatccctggtttgtgagaactgtaattccattacaacagtgaagaagttaccataactaatgaatggaaattagtcaaatgcctaattttttaggtttgctttaatttatttatctgtgagaaatgctaagcatgtcatgcgttgctatcttcaagaaatactaagaaactgcaaaggcaaagaatgtttgaaataacttaccccgcttgagtttctactgctgcaggctagatttcctgtcttgcagttgagcaaggtagctacatccttttcaagaagcattggtcgcccacaaatatcacaagctttctcagcagcaaggcgcttctgcttacgcaactccctcctcatagatttggtggataagaggccaacttgaagattgtgtgaagtacctgtcggggaacctgttatgatagcttggctattgtcatgggcggagctgctttgctcattcgactcctctgaagatgcttcttgatctgaaaatgacttctttcttctctttccacggtgtccagcatcatcaatcacgaagaaagatccagcagagataggaaggtcctgatcatcagaagaccacttcctgcccaactcaattgtataagagaagttgacaatggcaaagtcagattgctcataggtgtcacactcatccaagccatgggagccatcctgtcctacccaagcacaccagatcttgctaatctttttacttcctttgctagcttcccataacctgtatgcaatatttccatatcccaaaagatgcacaggcaaatccgaaacaacatcctttagcaatacactaggaataacgagaggaccgtcagttccactttggtttgacagcacatgatcttcagatacagaagcagttctaccattaccatgcgcatttgcaccacggcgtgtgccttttgcgccattgcgagagctagaatcatctctcaacctcgaagtcacttcagtgtcgttcgctggaaccagagccagctctctggtgttctgcgagctcgagtccagcaagagcgggtccttctcgcgcgagttg

SEQ ID NO: 21 - DNA, 1333 bp, Oryza sativa L.
  PCR marker G2155 MwoI
ccctctgcttgatccagtgtacatccatgggttaggacagattagttactcagttaattaagtgtgagactggaaaaaaatatctgacggcagttttataagttgagtgattgaactagtgaaagttcagttaactgtcaacggctgtagatttgggatggcagactgttctgagtcaaaatgaagcttttactgtgcgtggttaccaggtgcagtaaaataatttcagatctaatcgcagtaaaaaaatgtagtactatatgttaagacgagattggtcggtcaaaatctatctggccctttacatctcccaaatgttacctcagttgcaggtggtaaaaaaaaatcactcgtttcacgtgatgtcggcagatcatggaccatgtctcaaatgctgaaactctgaacaatcaacaaaaaaatccaaccagatgagctgtgcaactgataattgatcatcacactatttgcaactcatctttcatgtagatggaacttcaatcccgaagaaataatgacagcaaaatgctgcgatcctgaagaaaggatggcggcaaaatggcagcgataaaaaaaaaatggttggttactgaagaattatttgtgcagcagttgagacagtagcaagataagagctagctaagctagctaggtagagttggatggaagagtagtagtatgagatagagcatggagcgcgacaactcaagtggatgctaaagtaaaaggcattctcttctcttgtttggaatcagaaaagaaaagaaaagacttgagctgcttggctggaatgtttggttggatcatgcgcgctctccttagcttagctcgccaagaaatcctcgcttcatctctctcaataattcaaagccacgagctctctgctcatatccagtgcgacgattcccgttaatgcaaatgcattatatccagttcgaaatgttacaattcttgcgtttgcagcaagccagcaagtggtgtgaattgtttaatccctcgtgcatttcaacgaaattctctcacaaattcgcattgacttctttcttagcacaattagtaagcagtgacaaataaagaatttttgaacaggatgtctttccaaggaaggtgagattttttatgtggatagcaaggatcgcctttccttagcatgaagagaatgtgatcaactttacaccttgcttacgattatggccttaatttttgataccctaaacaggagcacatcacatgcatgtcgacctgagaccaccaattaactgattaagttggcatttcagatgcatccgtcagttacatgatcaggtgatcgatggatcaactgtaggtttca

SEQ ID NO: 22 - DNA, 863 bp, Oryza sativa L.
  PCR marker G291 MspI
cgaacaggatcaaaagtagacgacgagggcatttagaaggagaggaattgtatttgttcccggtatttaatttttaaatttgtggtcggaagtttcggaagaaaaaatgtgctcatgagtgattattggctctgaacaccaacctctcttttcgttgattccttctgaggtgttgggtgttgggacacgatgctgccgccgacacgacaccgggttccacaatacactaatctactcgcgacaccttcattgaactgcatataattatttagaaagtccattaacacatcttataaaaccttgttgaatcatataatcattctataaagtctatttgaacatcttatgaaaaaataagatctgacctagtcgttacactctcttacattttccattagcctaactaattccgtgcaggaaacgcccaaaaataatagtaccaatagtccactaatcccgtgccagaggccgccaatgattagtgattaacccaaaaaacataatcatcatcacacgccgctaatgaccagctctcgcttagctcatcccacaggcggcccccacacgccactcctgccatgtgggcccacctttcacaccccccaccaaccagaaaaaaaactcccccaaaaaaaaaacttttaatgcttatctcgcggcagtataaaaggcgaccccaccacccacacacaatcacagtcagcgacccaacccaacccgagccgaggagtcgagtcgtgtgaaaattacgaaattgcccttcgactccaccaccaccacccaccggcgaggcgaggagaggagaaaaattgggaggaaaaaaaaagggaaaaagaaaaagggtggaggagatttttgcgaag

SEQ ID NO: 23 - DNA, 1510 bp, Oryza sativa L.
  PCR marker R2303 BslI
tgccatgaagacctatggaaagaatatcttcttctcactctgtgaatggtgagtttactctctgtaacatttagggctaggtcgaaggaacatgaagcattgctgattcactccactgtgttttttttttctgtatagggggaaagaaaatcctgctacatgggcaggccgcatgggtaacagctggagaacaactggcgacatcgccgacaactggggcaggttctactcatcctctctttaaccctgtttacatagttcttgagtttttcagtactgatcgtaattgccctgttatttcagtatgacatctcgtgcagacgaaaatgaccaatgggctgcctatgctggacctggtggatggaatggtaagaacttgagatgtatctgttcctaggttgcttaaccatttgagagcttcaaaatgatcaacatatgtttctgctgtgcaatatcagatcctgacatgcttgaagtgggaaatggtgggatgtctgaagctgagtaccggtcacacttcagtatctgggcactagcaaaggtaccatagcatgttctatgtactaataattttgctgcaatgttgaacttctttgcatttcctcactgcaagttttgcttgaattgttcaggctcctcttttgatcggatgcgatgtgcgctcaatgagccagcagacgaagaacatactcagcaactcggaggtgatcgctgtcaaccaaggcaagccttctcagtttcacatgcttagatttagccatacctcttggatatttcaccatactcataatgtaactctctgaacagatagtctaggtgtccaaggaaagaaagtacaatctgacaacggattggaggtatcccttcaatggcttccaaatttgcagtttctcattgtcccataagccttggcatgatcatgactaactctgaagctgacaatactttgtgtaaatttgtcggtaggtttgggccgggccactcagcaacaacaggaaggctgtggtgctctggaacaggcagtcataccaggcaaccatcactgcacattggtcgaacatcgggctcgctggatcggtcgcggtcactgctcgtgatctatgggcggtaaagcctttgctttcttcagagctcaaagtagaacatcttctcttcagaattcagagttcataacaaatttctgtcaattgtgcagcactcttcgttcgcggctcagggacagatatcagcatcggtggcgcctcatgactgcaagatgtatgtcttgacaccaaactagtcagcaaagaaaagcagcacaggttagtacgtgtccggcgaatacagctaaattgatcaggattcaggaagaaggtttgcaatttgcaaggattggtagagctggaaatgggatgccatttggttatgtatgtagaaataagctgtaagcctgtaagcgtatatgtaatcagccgtcaaatgctggcgagtgtatttctgaagtttgcaacgaaagttgcagcaataaaaa

SEQ ID NO: 24 - DNA, 1016 bp, Oryza sativa L.
  PCR marker BstUI
tggggattcttttctttaagcaatttaacattattgtcctaacaatatacacaatattggtttttctttcagtatcaaataattcttttacttttgaaaacacatttgcaatgtgttggaaacacaattatatcttgcacttccttttggaaatttaatcatttgaaaactgattcgcgtttcatggctgtaatcttctcttgcgaacatcgctctttctttgatggttctctgttgagaagaagagcaaccaagtaaattttcgaaatgtttttttgttctttctattcaccattgcaggttgtcaaagccatcgagaaggccataccgattccgagagcgcaacccattgccttggatggcccagcaagggaagagctgaaggccatggaggcgcagaaggtcgagatcgaccgcaccgcggcgctccaggtgcgccgtgagctttggctggggctggcatacctcgtcgtccagactgccggcttcatgaggctcacattctgggagctctcatgggatgtcatggaacccatctgcttctatgtgacctccatgtacttcatggccggctacaccttcttcctccggaccaagaaggagccctccttcgagggcttcttcgagagccggttcgcggcgaagcagaagcggttgatgcacgcccgggatttcgatctccgccggtatgacgagctccggcgagcctgtggcctgccggtggttcggactccgacgagcccctgcagaccgtcgtcgtcgtcgtcgtcgtcttcgacgcaggagagccattgccattcttactgccattgccaatgatctttgtgctgttctgttctgttgtcagaattttttcatgcccagtttatgggggttaagctagcttctccattgtaccgttctgatgtgcggatgatgcgatgcaaagcatagtttgttgaagagatgacaaggcagattttagcttgaaaacctggaggtgagaaaaaaaaatcctgatgtgtttgtgtgtgtga

SEQ ID NO: 25 - DNA, 676 bp, Oryza sativa L.
  PCR marker S10602 KpnI
accaccttcatatgaagaaattaacggtgttttcatgaggaatccaacagtcgctgaattggtggaaactgtggaattcttcttggctgaggtaaccaatcatcacttcaccacaatgcacaagtttgtagcttactactacagtacttctaataagttttgtctgttgagattttattgctgatttctatgcatggtcatctttttgacaggccatccagtcttatcgtgctgagagtgaaactgagctcaacctggcagctggtgactatatagttgtccggaaggtacggccctatcttcccattggacatgtttctaaccataaacatatctttgctggacttttgtgggcaaagttggctacactaaacttgtgttcattaacctgctcaatcaggtgtcaaacaatggatgggcagaaggtgaatgcagagggaaagctggctggttcccttacgactacatcgagaaaagggaccgtgtgcttgcaagtaaagtcgcccaggtcttctaggcgttcaatgagccatacatacataaccctggtgttgtacactgtattatgatcgttcgtgatcttcaaagaccctctgatcagagaaatcacaaatattcttttgttctattattgtcattatcactaccccttttgtcaaaaccagtgcagcctttt

SEQ ID NO: 26 - DNA, 1059 bp, Oryza sativa L.
  PCR marker Tsp509I
gcgagatcatgaacttgattttctggttgccatattgggcttgcttgttaaccttgtagagaaggatagccttaataggtaagtccctcacatgcttccttccatttgctcaattcatatcagtgttactgttctggcagttccttggggtcaggactcagaaacatccaattaatgttcatgttctcttaacgactcagaaatactttataacctctccacagggtacggctttcatctgcccgtgttcctgttgatctatctcagaatccacagagtgaagagacacagagagatgtcatagcactcctctgttctgtattcttagcaagtcaaggtgctagtgaagcttctggaactatatcaccggtaattcaaaattcttcaagttccttttgtatgtagattatatctttgtaaaactcggcatttattacctgctctttgtttcaaaaagcagtattttattttgctccttagcataggtcagcagaacagttgatcttattcagaaaacaatattttgcatgtaacatactgttatctatgagatgaaaattaatgcatgtgtaataatgtcaatgataaatatttgctatctgaatccagtctaccaactctagttagaccgaattactgaggttctatttcaaagaataatttagtgcaccatttgttcaactactatgaagtaaaatggtattcccttctattgacatcgggttagaagtgaaaggccatcttaatgcgatgttctcaatgccacaaacccacaaatttcattaacacatacagattattattaacatagctataaattggatttccagaagcttgagttgaatttattttgttacaattgaaagcactgggaacattagcatttttttttagttcttggttattgcaatttataatgttatacagaactgtgtacctcacaatgcattcattatgacattctatgaaccatttgattgactgttgcttgtaaacaacaggatgatgaggagtctttgatgcaaggagcacgggaagctgaaatgatgatcgtagagg

SEQ ID NO: 27 - DNA, 76363 bp, Orza sativa IR24
gatcaactaacaacctctttgcagcaaaaaagcatacacacaagtgtttgtcttggcctggggctctgcagatggactgatactctgacctgcagtgggcttgggagctaacaatggtttcattctttttttttttatgttttcccctgttgtttttgctcatgttttgtgtaattttttcttctcatctagcgatgttatttttcttagcatgatgggagtagccctcctttttttttctctaattaagtgtaaagtagcaacagcatagggatgaatgttcagtgtagtgtgtggtgtttcagttattcagagacgtccatacagtttgtaccttgtgaccacacgtcttaatctgatgaagcttagaataaatcacatgttagcaatgcaatatcatctgcgtcttctctcactttggtggccatcaaattctgtgtagaagtgtatggttggtgtgctgttgcaaatgccgtattccgctctgttttgtggaagttaagaagtccctagttgaaataccgatttttcatgatctcggagattgatgcaactctgattgcagcatttctttttattagaatgtacactccatgctatcatgatgtttattgtttagtactacaagatttggttaaccattattttaatatcataataattttataaaatcttggagtaacaagttcataatacatgatagcataactttttgaggctagtctatgtatattgtctcctttgtttttaaactaagcactcaataaattattgatggctgtaattttctgaaggtttcaccggtttcggcccgtgctttataaatagcttcggcacaaaagacaaaacggtccctccaacacataaatggttgagtttacgttttcattatctttggtaaaatcaagtccaccacgtagacactcataacaaaagtttgaatatcctcagaaattttgacttgagtctatcttacctttgatatcggacatccaaccctccctccctccctgaactttatattattcatattacacctgaactttatattattcatattacaccctgaagtggttttcatttaattgcatacatgctgaaatagtttgacaacgtgagatgcactaaaaatctacacgttcgtcttaagttgcaattcattttatcccttttctttttctctcttacataggaatatcaatagtactaattcacattacaatatagtataaattggtaatcgattattggcaatatactatattaaatattcaaaactagtcatttaagctgccaaataagtaaaccactatcgaaaaccacaatataaatggcattacaaaacttagggggttgaatatccaattttaaagttcatgatgctagaggaatttctatcaaaagtttatgggtacatatggactttttcctttttaaaagaagctattcttgtcgtaaacgttaaatattttttgtactttattttttatgattgaaaaaaaaacttagttttcaaaatgattggtctgtatacaagcatcaattagacttaataaattcatctaacagtttcctggcagaaactgtaatttgtttttgttattagactacgtttattatttcaaatatgtgtacgtatatctgatgtgacaaccaaacccaaaaattttccctaactccatgaggccttacagatatatttgatgggtgtaaagttttttaagttctttgggtgcaaagtttttaaagtatacggacacacatttgaagtattaaatatagacaaataacaaaacatattacatattctgcctgtaaacaacgagacaaatttattaagcctaattaatctgtcattagcaaacgtttactgcagcatcacattgtcaaatcatagcgtaattaggctcaaaaatattcgtctcgtaatttacatgcaaactgtgtaattggtttttttttcgtcaacatttaatactccatgcatgtccaaatatttgatgcgatctttttggccaaattttgttggaatctaaacaaggatcaaatttgctgaatttttccagacgtcacggcttgttcatccatcgttcgcatcgcgattcgccaccgacgccttggtttccaacgaattttatcatccgcttaaatacatccaaagctctccatcgccatcggcggccaacggcgaccgctccgctctacccaatccacccatccactcgccgccgccccctgatccaaagcctccgccgcgccgccgtcgagaggaggaggaggaggaggaggaggaggcgtgagcccctatggggaccctcctccggccgcgtccgcttgcccacgccgccggcgccggcgacgccacgccgtcgaccgcgcacggtagccacgcgcctctcgagaggccccccccccccgccgctcgctgatctctcttctcatcctgtttgggtttgggtttgtgatttgggtgttttttttttttccgcagcggtggtggtgagcggtggccgcggccgtggcgtggagtgccagccgcatcgggtgcgccgccgcccgggtccgcaggttgcggtggcgacggcgagctggaggaggcggagggagaccgtggtgagatcggatttcgccgctggtggtgccgctaccatgggggattcgccgcaggcgctctcaggtttgcagcctcctccactctcttctcgcaaaatgtgttgctatgttcctctcgctgggctggcctcatagccattaatgtagtttgctggaacattacattcggaacgttgttggcaattgcttgacaaaatgtggaattgtggaggggagaaaaatcgtttgaacctgcagtgacaaaattgccatctataattttaaaactgaaggtgtggaaatcaaacataatcattgccagcacatcattcttgttaaccaccttgacatattgttggcttataacagttagctccacaccaacttggaaggtgtcaatggaatgtaagtataaattgaggataactggcagttgttaagactttctacagaacttgtagcagctaaaactagctattgtgcatttatgtttcatggaatttgagcggcaatggatatttcttactaagacgtataatgcaaaaaaaaaaaaaaaactatgtctatgcagtttacatgtaatgtgcggatgcaaataaaatcatgttcatggacaaactaatgggattcataccaaattccagaattgcatttcttatgtggttacttttgtttgttgatttggttaccagacatcgatgtggtttcaagggtcagaggggtttgcttctacgcggtgactgcagttgcagcaatctttttgtttgtcgccatggttgtggttcatccacttgtgctcctatttgaccgataccggaggagagctcagcactacattgcaaagatttgggcaactctgacaatttccatgttctacaagcttgacgtcgagggaatggagaacctgccaccgaatagtagccctgctgtctatgttgcgaaccatcagagtttcttggatatctatacccttctaactctaggaaggtgtttcaagtttataagcaagacaagtatatttatgttcccaattattggatgggcaatgtatctcttaggagtaattcctttgcggcgtatggacagcaggagccagctggtatggctgtagtctcatccctgctttcttaagtagacatatatacatttacagtatttggtaaataaacaagattttatgaatcatatatgattttggggaaaacacaaaactctctttgttggctgccttgaacatagttctgttcacacagttatagcaccttctttaaaatgaagaactttgttgcatacacataaggccaaaccacataatgaattttgtttatttctatctttgaatgttagcatcgtttttgtttaatgcatgatcgccttcctatatatttgtagtatgtcaacattgtattccatgctgagcataacaaatggtttgttaaaattcaggactgtcttaaacggtgtgtggatttggtgaaaaaaggagcatctgtatttttctttccagaggggactagaagcaaagatggaaagctaggtgcatttaaggttcagtaaccaaacttaggttacattacatctaatgagatttttatattcagtatataatgttaaccttctcatggtgtactgacgtggttataaatgtccccagagaggtgcattcagtgtggctacaaagaccggtgctcctgtgatacctattactcttctcgggacagggaaactgatgccttctggaatggaaggcatccttaattcaggttcagtaaagctcattattcaccatccaattgaagggaatgatgctgagaaattatgttctgaagcaaggaaggtgatagctgacactcttattctaaacggttatggagtgcactaaagaaagatggtgtttttttttattatatggaacctattcaaaggcacagacaggctttcaaggctaagcttgttacaggtactgatactagttactaattactttcgtaatcagtataaataagcttgtgtagtgtaatggcattgtacatttctgcacttggtaaatttacagaagaggcaagtaatattttagaggattgagtttattcacccagtcatatagttgaagaggcaagtaacctgtaagagaggactgaacattaacacctcttgttcgattaaaaatgaccaaagagcatcaaacatgtattcgaggctgttactttagatatggcccattaatttgtttagttgtctatgtacatcctagttggtgtaaatgccagttaccatttctatgatctaaaacaatcaactcttttagtatattttcaaaaacgaaattcagtacacatgtatgaatcttaatattcttctctagctcgttacaaaagcaacaaaggcaccgtgtcagctggttcacattagctagtttgtacttagcattatccactagcaccttattttcatgcatatcatgctaatttgcttgcccacgttgagtgggaatttttttcatgttttataatttatatatgttttagacttctagtccacaatttatgtacttcatgttcctgagcctctagtatggctgatagcagactaggtgctgagtgctgtccttttttgcagactgaagagagaagaaatacaagactgtccattgttagtcagatttgtaaaaatagactctgatgtagtttacttttgcccctattttatttttaacaatacaaatatataacagatcctaagaacttatcttaatttaggagaagttgctcgtttcattaaattaaattgtgaagtaaaaatgtgtgctcgagtctgtcaatgcaatcctgtgttcttgtttgaagatatggtgtagggcaggccaggattgaacactgaatggtaagactgcttctgccttcagacgttattgctaaatttttagctacttgcagttagtgctgccacgccgattaagcagtagaacaaagtagttttgtcgtgcacaaatgagttatatttcattggaaatcgaagcgaaaacgaatcaaaagttagaagaaaaggggaaacttggtaattactccataaagagagtgcattttattggtaagatggtatccggaagctgtgagctccgggctgtatgtattctggcaaatttgatatgagatgctcgattattggcttaagttagcgatatcaaatttggggaagcaccaaaggaattattgtgaaggagttatgggtgcgtgacgttatctgctaggttcaaatccttgtggctatgaatatttatctgctaggttcaaatcctagtgactatgaatattaatgggtaaggtaagggatttattgttaattttagtttctttaagattgtgccatcggacgccattcggtaactgtaataatgctttgtattggattcacttgtgttacatgcacgcactaaacatgtgctttaccttttcatctgtttttgcgttctgggctagaaactcaaacgttgaattttccatggtctgctcaacttgacaattactgcgtgtcaagcgatcttatacgcatactatgcgcacaagtgattgtatacggatatgatgacagtataacgtgtgatattgatttttttaataaaaaaatgatgttcatttccttgatgaaggaacaaagactttttttaaaagaagggtattactaaaaacaaaaatgacaaaaacaaaatatcagtgcacatggcaagtgtgctcggcaattttttctctgtactttaaacaaaaatacttctatatgttcttttttataagggtggcacaaatcttttaaatgagccaaatatctacattggatttattaaaaactgtataaattataatttatactctgaaaggttgtgtgcatctctcttggagaaaatgtataagttgcaaacaaacattaatccacgttatgtaactttttttcgccggaaaggccgaaggaggcctgacggagcgtggggctcctcaccgggagaccgcgcaggcccccctttgccggttcggccggggactcagggtgaaattctaagctctctgtatgtggaaggttcgcgaccgtcgaaagagcataagacacgggcgatgtatacaggttcgggccgctgagaagcgtaataccctactcctgtgttttgggggatctgtgtatgaaggagctacaaagtatgagccagcctctcccttgttctgggttccgaatctggaaaagtccagtccagtccccccctctaagtgggcaaggtcctccttttatatcttaaggggataccacatgcaccatctccctcctttctgtggagacttaccctaccttttcataaatggacggagatttgtatagttgccgtccgaatgaccttctgataggacggcccatacctacctccacttccgccgaaagcaggtgcgacgtgggattatggctgtctgctgacgacatgaccagtgtcagactggtcacaaattgctcattcctgtccaccacgcgtcagtttagcaatctacatgttggcccttcttcacacaacatcttgcctgtaatggttaggatgaagcctggcatatatctaaccaggactaacgtgccatctctaggaggtaacacgctagctccagctggggacgagcgcctagaagccctcgtcctgacgggatggggcgaggcgtgcgtcagatcgcctgtcgccacctaacctgcgatctgaccggtctgtgactggtcacagaccggataaacgagtgcactgcacttcgttacatgcagcgtgacacgctcagccaaaccgcaataaatgtggttaggtgagccccgctgtgctcacctaacccatacacgcggagcaaaaacccacgaggggtcggggcgcctcggccctcggggccgaggcgggtgcggtccgaccccctcggggggactaagaggagggcgaacacatcaccctcgggcccgacgtcccccgagggtgccaggccacgtgggcgattgtgtctgcctcaaacctctagtcatgatactcctgatcccatgtcaccgacagtagcccccggcgttatgccagggcgatcgccctctttaagggaagcggtcgggcgtgacgccactcctaaggcctggtgacaggtgggaccggtctccacaattgggcagaaacccaacggtcacaaatcacgcacatcggcaatggtaactctactatcaataatgagcggtctcttcaagactgccacattactcgagtagcacacgaatctggacatggcgattcgtttcgtctggagatatggtaacgtcgctttggtcggcgagcgtaattaacgcgcgcacgatatgatctatctcgactgccacaaccgcatatccacctcatgcgccgcaagcgggcgaatgggattagtggaagcgtgggcgcgagaaacgagggggcgaaatagtgggcgcgagaagcgaggagccgggcacagcgttggcaagagtataaaggcactgaggaaaggatctgtttccttcctttcgccatcatttcccttgtcttcgccgcttgcgccctaactccttctttcctgtgctctactttcgccacacgcgctcgctctcaatcttctcttcctccggcgccatggcacggggctccgctctgctcgatggtagcgtgctgccgccttcccgcatcgtgagcgagaggcaggctgggctgccgcgccgcttcatgccggaatctgccaccggccgggagatagtcacgctgggcgagggacgcccggcgccagactacccggggcggtccgtcttctttctcccctttgcaatggcagggctggttccgccattttcttctttcttcatggatgttctgaagttctacgatctccagatggcgcacctcacccccaacgcggtgatgacattggccatcttcgcgcatctgtgcgagatgttcattggggtgcgcccatctcttcggctgttccggtggttcttcaccgtgcagtcggtgtcgccgccatcggtagttggtggctgctacttccagccacgggggccggtgctgaatcgctacatcccctgcgccctccgcaagaagtgggacgactggaagagcgactggttctacacccccctcgccgacgaagcgcgcctccgacttccgagccagcccccggcgcaggcctccagctggcgggcgccggtagatctgggggatggctatgacgccgtcctcgaccgcctggcgggcctacgatcccaggggctcacagggaccatggtgtacggcgactacctccgtcgtcggattgcgccgctccagcggcgcgctcggggcgcctgggagtacaccgggtccgaagactacatgaggacccaccagggagtcagatgggactgggctcctgaggatttcaagatagtggtccaacgggtgctgaatctcaactccatggaggcgtccctcattccccaaggaatcctccctctctgcagcgatccagaccgcgcctccatcctgaccattatgacggcggtcggggcctcagaggagtgagctccaaagggccacgacggcgcaggcgggagccgtaggggggatcaatctacccggggagggggtcgtgcttctgggtctcgcgacggaggcccgaggagcagccgccctgccgacgcccgggggaagaggaagcagggaggaacacctcccccatctcctccccgagggggcggggcggtgcgtgccagcagcaggcgcccggagggggccgcgccgacatcgcagcccgagggggagcgcaagaagaagcggctccgcaagatgggggagacagaaccatctcagggaaaccttatttcccctctaaagtggtcgtttaaccgaccccctcgcaggttcgtctctcacccatcgtggctgtattcattctctcaacgcgagttttcactcacccatcttgttcgtcttctggtcttttcttctgtttcagcgagatcccgtcgcgtccctcccgccattccaagtccggccagtctgaggccgaggatccggcggccgcagaggcccggaggcgggaatctgaccggcgagaggccgcggatcgcctacgggaagccgaggaggccgcccaggaggccgcccgggctcgccaggtcgaggaaaccgctcgggaggaggccgcccgggcccgccaggccgaggaagccgctcgggaggaggccgcccgagcccaccaggccgaggaagccgctcgggagaaagccggatttcgccaggacgaggcaatggcgacttccgaggcagctcgcgatgaggtcgcgggcgcgtcgcttgagcccacttcctcgggcgacgctcaggcgacaacttccggggcagctggcgacgaggctgcgggcgcgtcgcttgggcccactccctcaggcgacgcccaggaccaaccaggtccgagggacatccctgagtccggcacttccatcggcggcccgagccgcgtggcatcctctccaaggcggctcttccccacgccttctatcgccccactgagcgcagagccccttctgcaggccttggccgccgcaaacaccgcggtgttggacgggcttagtgcccaggtggaggccctgcaagcagagtgggcggagctcgacgccgcgtgggcgcatgtcgaggaggggcggcgctcagtggaggccatggtggaggtgggccgcaaggcacaccgccggcatgtctcggagcttgaagcccgtaagaaggtgttggcggaaatcgccaaggaagtggaggaggagcggggggctgccctcattgccaccagcgtgatgaacgaggcgcaggacaccctccgccttcaatacgggagctgggaggcggagctagggaaaaagctcgacaccgcccagggggtgcttgacgctgccgctgcccgagaacagcgggcgggggagaccgaagcggcgtcccgacggcgcgaagagacccttgaggcgcgcgccatggcgctggaagagcgcgcctgcgtcgtggagagggatctggcggaccgcgaggccgccgtcactatccgggaggcaacactggcggcgcacgagtccgcctgtgccgaagaggagtccgcactccgcctccacgaggacgcgctcaccgagcgggagcgagctctcgaggaggccgaggccgcggcgcaacggctggcggacagcctgtccctccgcgaggcagcgcaggaggagcaggcgcgccgcactctggaatgtgtccgcgccgagaggaccgcactgaaccagcaggccgctgacctcgaggcgcgggagaaggagctggacgcgagggcgcgcagcgacggggcggctgcgggcgaaaacgacttagccgcccgcctcgctgctgccgaacataccatcgccgatctgcagggcgcgctaaactcgtccgccggggaggtcgaggccctccgcttggcaggcgaggtagggcccggcatgctttgggacgccgtctcccgcctagatcgcgccggtcggcaggtgggcctctggagagggcggaccgtaaagtacgccgccaaccatggaggcctcgcccagcgcctctcgaagatggccagggctctccaacggctccccgaggagctcgagaagacaattaagtcatcctcgagggacctcgcccaaggagcggtggagctcgtactggcgagttaccaggccagggaccccaatttctctccatggatggcgctggatgagttccctcctgggaccgaggacagcgcgcgcgcaggtccgggatgccgccgaccatatcgtccacagcttcgagggctcagcccctcggctcgcgttcgcccccaactccgacgaggaggacaatgccggtggtgcagacgacagtgacgatgaggccggcgacccgggcgtatcggattgatcccccaagcccccgccattctttagttttttcttcttttccttcttctaaggccttcgggcctcttttttgtatagatcaacttaatctgtaatcaaaaatgaagaaatttttgtgtcaatttcatcttgctgtgtgtatgagatgaggatgatctgtgacgtggtccttttgcgtcttagcttgattaagggctcgtgcccaggtcccagtcctcaaaaggcgtgggtcggggctagtgcctggggagatccacatgtcgagactggccaggccgggaacgtggtgaccgagggttatgggtgacccgattgtgggtttttgccgattcccccccggagttcaccacgccccggggcacggctcggttctgggccccgtttggcgattttagccgacccgagcccccgagggcaggattgagcacgagtgacctatttcaagtcaagattcttcaaaaggaaaaaaaaacacagatacagcctttaggaaattgaaactgcttttattgaaatactgaaataagagaaataagaatgtgcatgtgtggcagcccccggccaacgctgcacgcccgagggggtgcggggttggcccgagcccgaaacctgacacccgacccccccctcaggggtagaagcgacgaaggtgttcgatgttccacgggttaggcagctcaatgccgtcgcccgtggccagccgtatggagcccggccgggggacgccgaccactcgatacggaccctcccacattggtgagagcttgctcaatccagcacgcgtttggacgcggcgtaggacgaggtcgtcgacgcagagtgatcgggcccggacgtgacgctgatggtagcgccgcaggctctgctggtagcgcgcggctctgagggccgcgcgccgccttcgctcttccaagtagtcgaggtcatctctgcgaagttgatcttgatcagcctcgcagtacatggtggcccgaggagacctcagggtgagctcggatgggagaaccgcttccgcgccgtagacgaggaagaaaggcgtttccccggttgctcggcttggtgtagttcggtttgcccagagcaccgctagcaactcctcgatccatgaatcgtcgtgcttcttgagtatgttgaaggtcttggttttaaggcctttgaggatttctgaattggcgcgctccacttggccattgcttctggggtgggcaggtgaggcgaagcagagcttgatgcccatgtcttcgcagtagtcgccgaagagttcactagtgaattgggtgccattatccgtaataatacggttaggcactccaaaccgggccgtgatgcccttaatgaatttaagtgcggagtgcttatcgatcttgacgaccggataagcctcgggccacttagtgaacttgtcgatcgcgacatacagatactcaaacccgcccggggcccgcctaaacggtcccaggatatcgagcccctagacagcaaatggccacgaaagtggtatggtctgcagggcctgggccggctgatggatttgcttggcgtggaattgacacgctctacatcgccggaccaggtcgaccgcatcattgagagctgtcggccaatagaaaccctggcgaaaagctttaccaaccaaggtgcgcgaggcggagtgggctccgcattcgccttcatggatatcggcaagaagcacaacgccttgttcccgaggaatgcacttcaggaggattccattagccgcgcgccgatagagggtcccttctaccagcacgtagcgtttggagatgcgatggacgcgttcactcccttcgcggtcctcgggtaaagtcttatctgtgaggtatgcttggatctcggcaatccaagcaatcaatctaagggagctgggagcgctcccctcgggtcccgaggcctggacttcgacgggcctcgggggccggtcaggcgcgtccgtctcccctaaggggtcgggtcgcgccgacggctgggcaagcctttcttcaaaggcgcccggtggggtctgggctcgcgtggacgcgagccgtgagagttcgtcggcaatcatgttatcccgtctgggcacatgccgaagctcaatcccgtcaaaatggcgctccatacgccgtacttggcgcacgtaggcgtccatctgcgggtcagagcaccggtactccttacagacttggttaacgaccagctgggagtcgcctaacaccaggaggcggcggatccccagtccagctgccactctgagtccggcaaggagtccctcgtactctgccatattgttagtcgctcgaaagtcgaggcggaccaagtatctgaggacgtctccgctcggagaggtcaacgtgacccccgcaccggcgccctgaagagacagggagccgtcgaactgcattacccagtgggcggtgtgaggcagctgcgaggggtccgtgctggcctcggggattgagacgggctcgggagccggggtccactctgccacaaaatcggcgagagcctggctcttgatagcgtgacgtggttcaaagtgcaaatcgaactcagaaagttcgattgcccatttcaccacccgtcctgtaccctctcgattatgcaagatttgaccgagggggtaagacgtaaccacagtgacccgatgcgcctggaaataatggcgcagtttcctcgaggccatcagaatagcgtaaagcatcttctgggcctgagggtatcgggttttggcgtcccggagggcctcactaacaaagtagacgggccgctgcacctttcggtggggccgatcctcttcgctaggggccgcatccctggggcactcttcgtccaagcagcctcgcggggcgcacttgtcttctgtgctgatgacctcggggtcggaggataacaggggcggccttcccacagtggctttggggccgtcctgggggtcaggggctcctggcgtcgtcggacaagcgggcaaagggccaactccggtcgtcaggggccttaggcctccgttcggctcgggggcctcttctccctgctctttcccgggtcgagtcagcacagggttagcctcggggtcaaagggcgataggtgcggccttcccacagtggcctcagggccttcctgggggtcgggggctcctagcaccgtctgacaagcgggcagagggccaactccggtcgtcgggggcctcaggccaccgttcggctcgggggcctctcctccctgctctctcccgggccaagtcggcacagggtggggaagcgcgaaatgagaattatcctcatcgcgctccacaaccaatgccgcactaactacttgcggggtcgccgctaagtagagtagcaagggctcgtctggctccggggcgaccataactgggggagagcttagatacgccttcaactgggtgagggcattttcagcttccttcgtccaggtaaacggtccggagcgtttgagaagcttaaataagggtaacgccttctctcccagcctcgatatgaaccgacttagggcggccatgcaaccggtgacgtattgcacatccctaagtttgctggggggcgcatccgctctatagcccgtatcttctcggggttggcctcaatgccccgggcagagaccaagaacccgagaagcttgcccgcaggtacaccgaacacacacttatcggggtttaattttatgcgggcggagcggagactctcaaaagtttccgctagatctatgagtaacgtttcctggttgcgcgtctttacaaccaagtcatcgacataagcctcaatattacgtcctaattggctaccgaaagaaattcgagtagtacgttgaaaagtaggacctgcattctttaacccgaagggcattgtcgtataacaataggttcctatgggggtaatgaacgcagttttttcctcatcctccctagccatgcgaatctgatggtaaccagagtatgcatctagaaaacacaaaaggtcgcaccccgcagtggagtcgacaatctgatctatgcgaggcagggggtaaggatccttaggacatgccttgttaaggtcggtgtagtcgatgcacatccgaagcttgccgttcgccttgggaacgaccaccgggttcgccagccactcggcggggttgacgctgccatcatatttttcggcgatggtgggccggaaccttgggggccaacggacattccgaagactcgccacaaaggctctacagccgacaccaccaaccgggggcacggagggctgattcccgcgtccgtgttgaggtgacactctggacgaggaagcgccctccgttgcgtgggcagcacttcggtcattacgccggcgctcgatgctggtgcgggcgtccggccccccacgcagatctttctgggtcgaaggagtcgacgaaggagtggcggccgaatggcgaacagcggctgccgctcgtcgtgccctccgtcttgacgacgcggagccggtggtagcagcaccagaggccttggtggcggaggaccgcccaccagcatctaggcgctgccgtgccgtcatgactaatttggccacgtcgtccagccatcgttgggctggagactccgggtcagggacgacaggcgggtgacgtaagagcgcgcccgcagcttggagcgcgccctggggcgtgctgccgtcgccgtagacgaggaggcgacgctccccatctcgccgttcttctccatcgcccgcgatcggtgaagtcgcggatctttcgaccctctcgagcgcctccccccgcttaggactttggcatggagggagcggtggagtacgagctcgacggcgtgggttcggctccccgtcgtcgccactcacactcggagagaggtcgtgcgcctttgcttgctcggccatcaggctgaacaggaaaagcttggcgcacacggaagagtacgagagctcagaaaaacacacactgagtcccctacctggcgcgccagatgacggagcgtggggctcctcaccgggagaccgcgcaggcccccctttgccggttcggccggggactcaaggtgaaattctaagctctctgtatgtggaaggtttgcgaccgtcgaaagagcataagacacgggcgatgtatacaggttcgggccgctgagaagcgtaataccctactcctgtgttttgggggatctgtgtatgaaggagctacaaagtatgagccagcctctcccttgttctgggttccgaatctggaaaagtccagtccagtccagtccccccctctaagtgggcaaggtcctccttttatatcttaaggggataccacatgcaccatctccctcctttctgtggagacttaccctatcttttcataaatggacggagatttgtatagttgccgtccgaatgaccttctgataggacggcccatacctacctccacttccgccgaaagcaggtgcgacgtgggattatggctgtctgctgacgacatgaccagtgtcagactggtcacaaattgctcattcctgtccaccacgcgtcagtttagcaatctacatgttggcccttcttcacacaacatcttgcctgtaatggttaggatgaagcctggcatatatctaaccaggactaacgtgccatctctaggaggtaacacgctagctccagctggggacgagcgcctagaaaccctcgtcctgacgggatggggcgaggcgtgcgtcagatcgcctgtcgccacctaacccgcgatctgaccggtctgtgactggtcacagaccggataaacgagtgcactgcacttcgttacatgcggcgtgacacgctcagccaaaccacaataaatgtggttaggtgagccccgctgtgctcacctaacccatacacgcggagcaaaaacccacgaggggtcggggcgcctcggccctcggggccgaggcgggtgcggtccgaccccctcggggggactaagaggagggcgaacacatcaccctcgggcccgacgtcccccgagggtgccaggccacgtgggcgattgtgtctgcctcaaacctctagtcatgatactcctgatcccatgtcaccgacaaggccatccgaatgtattaaggagtaaaagttacaagaaaaaacaccataatgcaccaatgtgcatgaccacacaccatacactacccccaagcacaaaccactgagggtgaagcctagcaccaaacgaccgccactaagtgtgaccaaacgccgctaggcctacggcagcaacacatagatgagacttcgaaaacgatgccaccaaggtggtcacgacatctaggatgctgccatcgtccatctaaaaagatgtggttttcacccagagaaactcatcaagaaggggagagggtaacccttgacagcgccccaaggaggttacgacgcccgaaggcgtagccgctgccggtccggtgaaccaccggactaggcttccgcctaggaccctatagccttgatcgcagatcaccgtccaccactcagaaccaccacacagacaaaaggtagcacgtagcttccaccacaccgcaccgacgccccttcgtcggccgactccatcgaaccaccatccctgagagctggcccaggacccctccgttccaccacccgccggccgccttgccagttttggccaaaggagaacccgggactgggtgacattgcttcggcagcctgagcttcccccgctggcgagctgctgtctcaatccaacctagaaactccccgcaaaagaaggggatgagctctaggaagggcgagggtgccgaccggcaacgaggaagacaacccatcgactccagctccctttgcactaccatctggccctgcgccaatgccggatacgctgtcgctccggctccggcgccacccacctgcaccccctttgcctggtctccgcgcccctcctggctgcgtcgcgccgcccagctggccgctaagggcaccgcgacggccgcccggctaccgaggcctggccgcgccatgggacagctcgcgctggcaccagcgagccacggccgtcgcgctgttgccggcgccagcgagcacaaccgccagctccaagggccgagcatgccactgagccgccgccgctgccgcccgggccggctgcacgtcaccggcgcacacgaccgcacgccgccacgctccgcctccgcgcccgaggcagccccatgccattgccgcgcacctcgcccgcccgctgccgagccgccaccgcgcaccttgctgagccgccaccgccgtccctagccgcctcgtgccgccgccacgccagatccaggcgcgggatggccggatccggccttgggggcgccggatccaccgcctccccacaccgccacggcgtcaccacctccgaccgcagtgagggcttcgtcgtttgccccatcctcatcgcgtcgaggaggaagacgccaagaaaaaagggcctcgccgctgccttccttgctcgctgccggcttcgccgccggcgagctccggcggcggcgaggtgggggagaagaagtggggagtgggcagctagggttttttcgccccccaagccgcccgtgcgagagcgacggtgggggggggggggactttccaacctcttccagtgttctagttctccacgttatgtaactcaatttgtttaaccatagaaagtaagaaacctaccagcgtgttaagctctctttcattccctttcttcttcctggttttgcttccatcacatgtcaagtgaagggttcttaactaccattactcctacacatctaatttttttctcagatctttcgcaggtatatattgatgctacattttatgatcttaagataatctccttcacattaccctctgctgaaactttagcttgaaccgtcatcttcaccacaatttgagcccaatttgcacagagcacaacgagcaatagcttgcccttacgttcattatttagcatgaactactactaactacccaagaatcaatacaccggtttaataacgccattttatcacgttaatatatgtttcattcaacacaccggttttggcacagttgcaaacttgcaataaattctttcctacttctccatcccataatataacaaattggtatgtctcgtctggtactaagttactatattatgagatggagggagcacttcttttcttccaaaatataagaatatagtattggattagatattatctagattcacgaattcgattaggttgtctagatttatagttgtatgtaatgtataattcggtaataggttattacctctcaggatggagggagtagttttgactttttttttcttataaatcgctttgatttttatattagtcaaattttatcgagtttaactaagtttatagaaaaaaattagcaacatttaagcaccacactagtttcattaaatttagcatggaatatattttgataatatatttgttctgtgttaaaaatgctgctatatttttctataaacgtagtcaaatttaaataagttagactaaaaaaaatcaaaacgacttataatatgaaatggaggaagtagtagactataacaaatttaaaccgtgctttgattttagagcatcactaatatgttagcaataatctatccctaaaatttattttttttcctaaactgaaaataggaagtggaaatactcctccatctaagagagagcctaaattcaataaaaaactaaaaaactaaaggtggatccctctattaaactaccgcaaaaaatttatgttttttttctcttccacgcgcgcagaacagatatctcgatcaagttagcatgtaaaatttttaaagagataccttatacgactccttccgtatttccaaaagcaaacggatttaaaatctgactcaaataaagatctatatatccaatttacatgacacatgtttcgccgaatttttatattaataataattaatatttttaaaattaaattattagcaatttgtttggaggatttatcaaaacaggatggacgttgtttataacagcgtctagacctagacgcgcttgcaaactgcggccacccttttatcacacaaatttttgacaatttgacactttccaaaaattaattttataaattaaccgtgaccaaaacttatttaaaaatgatctttttgttgagcgcaaaatcgtatacttcagcgccaaatagcacggcgccgacctcccccttcccctcccctctatcctccactgctgccgcccacctctccgtatcagctgcgtcgcgttggtttccgccggcgctgctgctgctgcaccagtccgctagggcgggcgggcatggcgcgccgcgccgcttcccgcgtccgcgccggcgctgttggcgcccttcgctcggagggctcgacccaagggcgagggggccgcacggggggcagtggcgccgaggacgcacgccacgtgttcgacgaattgctccggcgtggcaggggcgcctcgatctacggcttgaactgcgccctcgccgacgtcgcgcgtcacagccccgcggccgccgtgtcccgctacaaccgcatggcccgagccggcgccgacgaggtaactcccaacttgtgcacctacggcattctcatcggttcctgctgctgcgcgggccgcttggacctcggtttcgcggccttgggcaatgtcattaagaagggatttagagtggatgccatcgccttcactcctctgctcaagggcctctgtgctgacaagaggacgagcgacgcaatggacatagtgctccgcagaatgacccagcttggctgcataccaaatgtcttctcctacaatattcttctcaaggggctgtgtgatgagaacagaagccaagaagctctcgagctgctccaaatgatgcctgatgatggaggtgactgcccacctgatgtggtgtcgtataccactgtcatcaatggcttcttcaaggagggggatctggacaaagcttacggtacataccatgaaatgctggaccgggggattttaccaaatgttgttacctacaactctattattgctgcgttatgcaaggctcaagctatggacaaagccatggaggtacttaccagcatggttaagaatggtgtcatgcctaattgcaggacgtataatagtatcgtgcatgggtattgctcttcagggcagccgaaagaggctattggatttctcaaaaagatgcacagtgatggtgtcgaaccagatgttgttacttataactcgctcatggattatctttgcaagaacggaagatgcacggaagctagaaagatgttcgattctatgaccaagaggggcctaaagcctgaaattactacctatggtaccctgcttcaggggtatgctaccaaaggagcccttgttgagatgcatggtctcttggatttgatggtacgaaacggtatccaccctaatcattatgttttcagcattctaatatgtgcatacgctaaacaagggaaagtagatcaggcaatgcttgtgttcagcaaaatgaggcagcaaggattgaatccggatacagtgacctatggaacagttataggcatactttgcaagtcaggcagagtagaagatgctatgcgttattttgagcagatgatcgatgaaagactaagccctggcaacattgtttataactccctaattcatagtctctgtatctttgacaaatgggacaaggctaaagagttaattcttgaaatgttggatcgaggcatctgtctggacactattttctttaattcaataattgacagtcattgcaaagaagggagggttatagaatctgaaaaactctttgacctgatggtacgtattggtgtgaagcccaatatcattacgtacagtactctcatcgatggatattgcttggcaggtaagatggatgaagcaacgaagttacttgccagcatggtctcagttggaatgaaacctgattgtgttacatataatactttgattaatggctactgtaaaattagcaggatggaagatgcgttagttctttttagggagatggagagcagtggtgttagtcctgatattattacgtataatataattctgcaaggtttatttcaaaccagaagaactgctgctgcaaaagaactctatgtcgggattaccgaaagtggaacgcagcttgaacttagcacatacaacataatccttcatgggctttgcaaaaacaatctcactgacgaggcacttcgaatgtttcagaacctatgtttgacggatttacagctggagactaggacttttaacattatgattggtgcattgcttaaagttggcagaaatgatgaagccaaggatttgtttgcagctctctcggctaacggtttagtgccagatgttaggacctacagtttaatggcagaaaatcttatagagcaggggttgctagaagaattggatgatctatttctttcaatggaggagaatggctgtactgccaactcccgcatgctaaattccattgttaggaaactgttacagaggggtgatataaccagggctggcacttacctgttcatgattgatgagaagcacttctccctcgaagcatccactgcttccttgtttttagatcttttgtctgggggaaaatatcaagaatatcataggtttctccctgaaaaatataagtcctttatagaatctttgagctgctgaagccttttgcagctttgaaattctgtgttggagttcttttctcctacagtcgtattagaggagggatcttctctttatgtgtaaatagcgaggtatgtatgtcacctctccgaattatttttactctggttcctagacggtaaacaagcaattatgttctgcctttgatgccagaaaaaacacaaaagtttgtcgttatctctactaacggatcataaaggaatttgtaactggagtttcaaacttaatttgtctaggcagtagttttggcattagatccaacattgtgtaggattcatttgtgtgtatcaatctatagggtttcattaaatttcgtttatgtgtactgtttaggtgttgaatagtttgacttgttttttaactgaacaaaagatactgaaatcgttccattcaacaaacacatgttccgttaatgaaattattgtacgttaccttttgttttcttactcacaagtgtcctcttttcttatatcctatagattggtacaacaaattattgattcaattttggttttgaacattgatgatcctccctgcactattggtgcagctgctcttctattcattttgtgaagtgatgtgagtacctctcaatcccatccttatgcttctgtgcatgcttcattccaattttttacgcatatcgattgttttcttttatataacagtccataaagataatcacatcatgacaaagttatttatttctacagtatagttatataagtattcaccagttttccatgaatattttggcatgtgattacaaagaagattatttgagaaaatccatgcttttatttcatcattttgtttgaagttgaactttaatttatggtgtaaatttcagttattattgctagcagctcgtactctttaatggtataacttcacttgtgcttattctccaatatctcccttcttgttgttcaggttcaagaaaatcatttgttggattcagaatctggtgtccattttcttcttaaattattaaatcctccagtgaatcttgttgattccaaagcaccatcgataggttccaaacttcttggaatcagtaaagttcaaatgcttaatggatcaaataaggattctgactgcatttcagaggaaatcctttcaaaagttgaagagattctcttaagctgtcaagtgatcaagtcgctcgacaaagatgacaagaaaacaacaaggccagaactgtgtccaaagtggcttgctttgttgacaatggaaaatgcatgcttgtctgctgtttcagtagagggtaagttttaatcaaatttcttggtcatgatttccctttatgaccattatatttatttatatgagccaaataagcagttgtcaacttgtcataagttacatagcacctatttgcaatattcatgggtggtttgcttagcccttttcttcacctgcttttgattgatgacttccatctgtgttgcagaattgaattggagtagtggactgcactagaagcacctatggccattgtcatactaggaaggttttcccttatcaaatatttgattgttacagagacttctgacacagtgtccagagttggaggaaattttaaagagacattaagggagatgggaggtcttgatagtatttttgacgttatggtggattttcattcaacattggagatgagatctcgctaacatcgcatattttacatttcctttgttcaactctaatagattgtgcaggcttgttccttttcgccattttagctttaatgcgcttgaagccacatgaaagtaatgcttgtccagatacatagccaaaggttgttatattttggggcatggaaaatgcttgaggtagtaactattttcatcaggacatggaaaattggctgcaacacaaattatgttgttttatgttgcaaaaatagttttttaatacttttttattctgcatgtggtgttagtatcttacagttcctctgatgattatatcccccacgataataacacttgaaacgataataacacttgacatatctacaccaagtgaacattattcatttggatgttacttttccagctatacttgctgttcttgcatgtgtaagcaagtttggagtaaattgcgcattaatttaaatgcttggtgttcctatctgtgtactttttattccccaactaataatgcaatcatattacgctgataaactgaataaataaattaacaatatacttctggtggcaaaccttgtgtatcagaatctcataaaggatacatccacttcagctttggaccgaaatgaaggaacatctttgcaaagtgctgctctcctcttgaaatgtttgaaaatattggaaaatgccatatttctaagcgatgataacaaggtaatgctccttatatgttctgtttcagtttagtacccatttccttcttctgtactatcttctctcctgatttgttctgtgcaaaatgtgcaaacagtgcgactttgtatgtctgcttaacaattttcttttcttcctgaaaaagcaatatgaactcttacattcattttgcttcttgcagacccatttgcttaatatgagtagaaaattgaacccgaaacgctccttgctttcttttgttggtgtcattatcaatactattgagttattatcaggtatttttcttaataatacaatgtgttcgctaacacaataaaatgttttaaacatccagtatgttaaagttgcagtctgacgcctattttgttttgctgcagctctttcaatacttcagaattcttctgttgtttccagctctacatatccgaaatcgtctaaagtctctcaacagagttactctggtaataacaaacaccaattttgtttgatcagttgatctcgttggcttttctatgcactgtctcaatatagtttggtcgccattcaagtctcactacagatgttgaacttggcctgacaccaaatatttataaaatgctacctgatatttttaatatttcatgtttcctgacccagattatcttgttggttcctcgtataagtttaattagtgacattcttgaagctttgttatgcagcagatgtcatggggggaacttcatttaatgatggaaagagcaagaactcgaaaaaaaaaaacttttgtcgaaccagacacgtcattgttgcttatcttcaaaatcagaagtttctcatattactatatcttctggtagtgatgctggtctgtcacagaaggcattcaattgttctccatttatatcaagcaatggggcatcaagtggttcattaggcgagaggcacagcaatggtagtggtttgaagttgaatataaaaaaggatcgtggcaatgcaaatccaattagaggctcaactggatggatttcaataagagcgcacagttctgatgggaactccagagaaatggcaaaaagactccgtctatcttaaaatgtaatcaccgacagtggtggtggtgatgacccttttgcatttgaccgccgcgtcggcgtcgccaccacgtaatcgcccacgtcgctgcccccgctgccacgtcgtcgaccgcgcacggtaatcacacgcatctcgaggccgccgctagctgatatcttctcatccggttgatttgtgattttggcgtttttgcagtggtgatggcggggggcgaccgtggccgaggcgtggagtgccatccgcatcagggtgtatcggccgcgctgctccgccctggtccgcaggctttggcggcgagctggcggcggagggagactgtggtgagatcggatttcgccgctggtggtgtcgctaccatgggggattcgccgcaggcgctctcaggtttgcagcctcctccactctcttcccttttttatttttttttctcgcaaaatgtgttgtgatgttcgtctcgctgggctggcctcatagccattaatgtagtttgctggaacatttacatttggaacgttgttggcaattgctttacaaaatgtggaattgtggaggggagaaaaatcatttgaacctgcagtgacaaaattgccatctctaattttaaaactgaaggtgtggaaatcaaacataatcattgccagcgcatcattcttgttaaccaccatgatatattgttggttataacagttagctccacaccaaccttgaaggtgtcaatagaatgtttagtataaattgaggagaacaggcagttgttaagactttctaaagaacttgtagcagctaatactagctattgtgcatttgtgtttcatggaatttgagcagcaatggatatttcttactaagatgtatgatgcaaaacaaaaaactatgtctatacagtttacatgtaatgtgcggatgcaaataaaatcatgtacatggacaaactcatgggattcataccgaattccagaattgcatttcttatgtggttacttttgttgttgatttggttaccagacatcgatgtgatttcaagggtcagaggggtttgcttctacgcggtggctgcagttgcagcaatctttttgtttgtcgccatggttgtggttcatccacttgtgctcctatttgaccgataccggaggagagttcaggaaaaaaatttgaaaatacccattttttgaaaaagatttacgtttatatacactagtatgaagaatttgcgaaaatataactaatccgcagatcggttatgcgggagcgcaacaaaagtatggcgtggcggcgcggagtggacggccgaggcgttcgcgcggaatggggctgcgggaccgagccagtctcgcttgccggtaacgcggaaccggtacgctcccgcagcgccagtgtgcggaaccgcggcgccaacatttttttactgcatggcactgtgtttaatactgtttgacactgtttctggtactgttttacacagttcccgggtcagttccgcacaatggaggcgcggcaccgaccatgaacaatgtgtgaacagtgctgcacagggttaaaacagtgtataaactgcgctgcacagtgctggagtcgctggccactgcggttccgcgttttggaaccgcgggaccgtcgcgattccgcgttttggagctgccggaccatgacggttccgcgcaggatcgtcggtcccgtattttgaatctgcggaaccgtcgctgtcccgcgtttccatttcgcgggatgcgtatatttttataaaacctctccatgcatgtatataaacataaattattgaaaaaataagtatatttgcaaatttttttcgagagctcagcactacattgcaaagatttgggcaactctgacaatttccatgttctacaagcttgacgtcgagggaatggagaacctgccaccgaatagtagccctgctatctatgttgcgaaccatcagagttttttggatatctatacccttctaactctaggaaggtgtttcaagtttataagcaagacaagtatatttatgttccgaattatttgatgggcaatgtatctcttaggagtaattcctttgcggcgtatggacagcaggagccagctggtatggctgtagtctcatccctgctttcttaagtagacatatatgcaattacagaatttggtaaacaaacaagattttatgaatcatatatgattttggggaaaacaccaaactctctttggtggctgccttgaacatagttctattcacacagttatagcaccttctttaaaatgaagaactttgttgcatacacatatggccaaaccacataatgaattttgtttatttctatctttgaatgttagcaccttattttcatgcatatcatgctaatttgcttgcccacgttgagtgggaatttttttccatgttttataatttatatatgttctagacttctagtccacaatttatctacttcatgttcctgagcctctagtatggctggtagcagactaggtgctgagtgctgtccatttttgcagactgaagagaggagaaatacaggactgtccgttgttagtcagatttgtaaaaatagactctgatgtagtttattttagcccctattttatatttaacaatacaaatatataacgtatcctaagaacttatcgtaatttaggagaagttgctcgtttcattaaattaaactgtgaagtaaaaatgtgtgctcgagtctgtcaatgcaatcctgtgttcttgtttgaagatatggtgtagggcaggctaggatcgaacactgaatggtaagactgcttctgccttcatttgtgcacttggtgctgccacgccgattaagcagtagaacaaagtaattttgtcgtgcacaaatgagttatatttcattgaaaatcgaagtgaaaatgaaccaaaagatagaagaaaaggggaaacttggtaattatatactccacaaatttattggtaagatttgatattagacgctcgattacttggcttaagttaaggatatcaaatttggggaagcaccaaaggaattattgtgaaggagttgtgggtgcataacgttatctactagttcaaatcctagtgactatgaatattaatgagtaaggtaagggatttattgttaattttagtttctttaagattgtgtccgagtacaccattcggtaagtgtaataatgttttgtattggattcacttgtgttacgtgcatgtgcttttaccttttcatttgtttctgcgttctgggtatgaatttgacgagattccatggtcagctcaacatatcagttactgcgtgtcaagcgatcttatatggtatgcgcacaagcgattgtatacggatatgacagtataatgtgtgatattgatacgatgttcctttcctttataaaggaacaaagactttttttaaaaaaagaaggggtattactaaaaaccaaaatgtcaaaaacaaaatatcagtgcacatggcaagtgtgcacgagcaatagcttgcccttacgttcattatttagcatgtactactactaactacgcaaaaatcaattcaccgattattaaactgttaacatcattttagcacgttaacatatgtttcattcaacacaccggttttggcacatttacaaacttgcaaagttgcaatactcccttcgttacatagcataagagattttaggtgaatgtgacacatctatccaaattcattatactagaatgtatcaccgcctccacgccgggagggagagcgccgccggtggagaaagggggagggagtggtcgaggggaaccagtagggtgccctccccgtcgccgcctccccgtggccgcgccggcgagacaggaggaagagggggagatggagcggcgccgccggtgagggcgcgcgtgcgcgggggggggggggggggagcggcgacgccggtgaggaagggaaggggagtggtggctttgagagagataggggagagggaaaatgattttagagttagggtttgggctgctgagtttttatatagatcgggatcaatcaggaccgtccatcagatcggacaactacggtttctcccgcgttgggccgggtgccactcctaggttgcccacactattgggccacatgtacgctccgcgtgaaataagttcactttaggtcctttaagttgcctctgaattgttcccaggccggccgcactattgggccaccccataggccatgtgtacgctccgcacagaataatttcgctttagctcccttaatttgtcccctcaaacttctaaaaccagtgcaaatctttaatttttagttcacccattgcaactcacgggcatatttgctagtgacatataatatgaaacgaaggatgtagcagactatagaatttaaactgtgctttcattttagagcatcactaactgttatttagatttttatttaaataaatgcagaaatgatgtttttattatgaaaattagcaataaagctcccaaaatttcaaaaaaaaattaaaagagatttattaatcatggttaatttaattaaaaattaaatctaaccatatcatattatttcacggtccgtgatgaggaaatggcagctgctatcacttatggtgggagagaaggggcattgtttatttttataactatctcttataactcccatgaaactataaaataaatataatcattatcataacattagtttttttccattgcaacgcaagggtaatttttcagtacaataaaaaaataaaagtgggccattctgaacggaaatttctggttttttttcccaagagcgccgcacacaactgcgcaagagatcgatcgcgatcaccctgctcgtcgccgatctcctacaccatccctgccatctccttcccctccactggctgctgctgcacctgtcagctagggcgggcatggcgcgccgcgccgcttcccgcgctgctggcgcccttcgctcggagggctcgatccaagggcgagggggccgcgcggggggcagtggcggtggcgcggaggacgcacgccacgtgttcgacgaattgctccgtcgtggcataccagatgtcttctcctacaatattcttctcaacgggctgtgtgatgagaacagaagccaagaagctctcgagctactgcacataatggctgatgatggaggtgactgcccacctgatgtggtgtcgtacagcaccgtcatcaatggcttcttcaaggagggggatctggacaaaacttacagtacatacaatgaaatgcttgaccagaggatttcgccaaatgttgtgacctacaactctattattgctgcgctatgcaaggctcaaactgtggacaaggccatggaggtacttaccaccatggttaagagtggtgtcatgcctgattgcatgacatataatagtattgtgcatgggttttgctcttcagggcagccgaaagaggctattgtatttctcaaaaagatgcgcagtgatggtgtcgaaccagatgttgttacttataactcgctcatggattatctttgcaagaacggaagatgcacggaagcaagaaagatttttgattctatgaccaagaggggcctaaagcctgaaattactacctatggtaccctgcttcaggggtatgctaccaaaggagcccttgttgagatgcatggtctcttggatttgatggtacgaaacggtatccaccctaatcattatgttttcagcattctagtatgtgcatacgctaaacaagagaaagtagaagaggcaatgcttgtgttcagcaaaatgaggcagcaaggattgaatccgaatgcagtgacgtatggagcagttataggcatactttgcaagtcaggcagagtagaagatgctatgctttattttgagcagatgatcgatgaaggactaagccctggcaacattgtttataactccctaattcatggtttgtgcacctgtaacaaatgggagagagctgaagagttaattcttgaaatgttggatcgaggcatctgtctgaacactattttctttaattcaataattgacagtcattgcaaagaagggagggttatagaatctgaaaaactctttgacctgatggtacgtattggtgtgaagcccgatatcattacgtacagtactctcatcgatggatattgcttggcaggtaagatggatgaagcaacgaagttacttgccagcatggtctcagttggaatgaaacctgattgtgttacatatagtactttgattaatggctactgtaaaattagcaggatgaaagatgcgttagttctttttagggagatggagagcagtggtgttagtcctgatattattacgtataatataattctgcaaggtttatttcaaaccagaagaactgctgctgcaaaagaactctatgtcgggattaccaaaagtggaaggcagcttgaacttagcacatacaacataatccttcatggactttgcaaaaacaaactcactgatgatgcacttcggatgtttcagaacctatgtttgatggatttgaagcttgaggctaggactttcaacattatgattgatgcattgcttaaagttggcagaaatgatgaagccaaggatttgtttgttgctttctcgtctaacggtttagtgccgaattattggacgtacaggttgatggctgaaaatattataggacaggggttgctagaagaattggatcaactctttctttcaatggaggacaatggctgtactgttgactctggcatgctaaatttcattgttagggaactgttgcagagaggtgagataaccagggctggcacttacctttccatgattgatgagaagcacttttccctcgaagcatccactgcttccttgtttatagatcttttgtctgggggaaaatatcaagaatatcatagatttctccctgaaaaatacaagtcctttatagaatctttgagctgctgaagcattttgcagctttgaaattctgtgttggaattcttttctcctacagtccgattagaggagggatcttctctgtatgtgtaaatagcgaggtatgtatgtcacctctccgaattattttgactgtggttcctggactgtaaacaagctattatcttctggtgttgatgccagaaaaaacacaaaagtttgtcgttatctctactaacggatcataaaggggtttgtaactggagtttcaaacttaaggtatctaggcagtaggtatatattgatcctacatcttatgatcttaagatgatatccttctcattatcctctgctgaaactttagcttgaaccgtcatctacaccacaatttgagccccttagcacagagcacaacgagcaatagcttgcccttacgttcattatttagcatgcactactactaactacccaataatcaatacatcggttattaaactgtttgtacagtttaataatgtcattttatcacgttaacatatgtttcattcaacaccacaccggttttggcacagttgcaaacttgcaataacatttttactacttctccgccccataatataacaatctcgttccatactatattgctatattacaggatggatgaagtacttcttttcttccaaaatataagaatctagtactagattagatattatttggattcacgaatttgattaggctgtctagatttgtagtcgtatgtaatgtctaattcggtaataggttattacctctttggatggagggagtagtttttatttcgtactccctccgtttcatattataagttgttttgacttttttcttagtcaaattttattgagtttgattaaatttatagaaaaaaattagcaacatttaagcaccacattagtttcattaaatgtagcatggaatatatttttataatatgtttgttttttattaaaatgctactatatttttctataaatgtagtcaaatttaaagaagtttgattatgaaaaaatcaaaatgacatataatatgaaactgaggatgtagcagactatagcaaatttaaactatgcttttattttagagcatcaccaaaagattagcaataatttatccctaaaattcaagttttgggtttcttaaactgaaaataggaagtgaaaaatcttttccgtccaagagatagcctaaatcttatcttaactaattaaaatattcataattttcctttcgtcacattaaattttcgtccgtaaatctgattgaaatccaattggacaatccaaaaaatagagaaaaagaacagaaaaaataataaaaagcacacaaatcttatctcaatcccgcgggaagctgccgacgccgccgaatccgctcgagcgccgccgccgccgctcacggggaacgatgtcgctgctgtcgcacgcggtatgggagggcgccgctgccactgcttgggagataggatatggagagagaaggaaatgtgagggttagggttaggtttttccccgtccgtatcttcagcgacacggaggcgatccaagctgtccatcagatcggacggctcagaatgcctccatcgtcgggccgcgcatgcttgatgggccgagggaaggccggagggtcgaacaaacgcaatcaaaggaggagttggaggaggtaaattagaatttatttgcgggctgagatagtaaatggactgaaaatggcccatagagaaattgggaattttatttaaataaatgttgaaaaggtgtttatattatcaaaattaaaaattaagctccgaaaattctaaaaaatattcaaagagcattattaatcatggttaatttaataaaaattaaatccaaccatatcatattatttcacggcgcgcggtaggaaaatgcgcagctgttgtcgtttacggtgggagagaagggacattgtttatttccagaactatcttttataactcccatggaactttaaaataaatataatcattattatagcattagtttttttctgtcttttttttccccaagagcgccgcgcagaagagatcgatcgcgatctccctgccccgacgtcgccggccgatctctcattctctccacgccctgctcgtcgccgatctcctacaccatccctgccatctcctccttcccctcccctctatcctccactggtgccgcccacctctccgtataagacaaactgcgttgcggcgttggtttccgccggcgctgctgctgcacctgtcagctagggcaggcatggcgcgccgcgccgcttcccgcgctgttggcgcccttcgctcggacggctcgatccaagggcgaggaggccgcgcggggggcagtggcgccgaggacgcacgccacgtgttcgaggaattgctccggcgtggcaggggcgcctcgatctacggcttgaaccgcgccctcgccgacgtcgcgcgtcacagccccgcggccgccgtgtcccgctacaaccgcatggcccgagccggcgccggcaaggtaactcccaccgtgcacacctatggcattctcatcggttgctgctgccgcgcgggccgcttggacctcggtttcgcggccttgggcaatgtcgtcaagaagggatttagagtggaagccatcaccttcactcctctgctcaagggcctctgtgccgacaagaggacgagcgacgcaatggacatagtgctccgcagaatgaccgagctcagctgcatgccagatgttttctcctgcaccattcttctcaagggtctgtgtgatgagaacagaagccaagaagctctcgagctgctgcacatgatggctgatgatcgaggaggaggtagcgcacctgatgtggtgtcgtataccactgtcatcaatggcttcttcaaagagggggattcagacaaagcttacagtacataccatgaaatgcttgatcggaggatttcaccagatgttgtgacttacagctctattattgctgcgttatgcaagggtcaagctatggacaaagccatggaggtacttaccacgatggttaagaatggtgtcatgcctaattgcatgacatataatagtattctgcatggatattgctcttcagagcagccgaaagaggctattggatttctcaaaaagatgcgcagtgatggtgtcgaaccagatgttgttacttataactcgctcatggattatctttgcaagaacggaagatccaccgaagctagaaagatttttgattctatgaccaagaggggcctagagcctgatattgctacctattgtaccctgcttcaggggtatgctaccaaaggagcccttgttgagatgcatgctctcttggatttgatggtacgaaacggcatccaccctgatcatcatgtattcaacattctaatatgtgcatacgctaaacaagagaaagtagatgaggcaatgcttgtattcagcaaaatgaggcagcatggattgaatccgaatgtagtgacgtatggagcagttataggcatactttgcaagtcaggcagtgtagacgatgctatgctttattttgagcagatgatcgatgaaggactaacccctaacattattgtgtatacctccctaattcatagtctctgtatctttgacaaatgggacaaggctgaagagttaattcttgaaatgttggatcgaggcatctgtctgaacactattttctttaattcaataattcacagtcattgcaaagaagggagggttatagaatctgaaaaactctttgacctgatggtacgtattggtgtgaagcccaatgtcattacgtacagtactctcatcgatggatattgcttggcaggtaagatggatgaagcaacgaagttactctccagcatgttctcagttggaatgaaacctgattgtgttacatataatactttgattaatggctactgtagagttagcaggatggatgacgcattagctcttttcaaagagatggtgagcagtggtgttagtcctaatattattacgtataacataattctgcaaggtttatttcataccagaagaactgctgctgcaaaagaactctatgtcgggattaccaaaagtggaacgcagcttgaacttagcacatacaacataatccttcatgggctttgcaaaaacaatctcactgacgaggcacttcgaatgtttcagaacctatgtttgacggatttacagctggagactaggacttttaacattatgattggtgcattgcttaaagttggcagaaatgatgaagccaaggatttgtttgcagctctctcggctaacggtttagtgccagatgttaggacctacagtttaatggcagaaaatcttatagagcaggggttgctagaagaattggatgatctatttctttcaatggaggagaatggctgtactgccaactcccgcatgctaaattccattgttaggaaactgttacagaggggtgatataaccagggctggcacttacctttccatgattgatgagaagcacttttccctcgaagcatccactgcttccttgttatagatcttttgtctgggggaaaatatcaagaatatcatagatttctccctgaaaaatacaagtcctttatagaatctttgagctgctgaagcattttgcagctttgaaattctgtgttggaattcttttctcctacagtccgattagaggagggatcttctctgtatgtgtaaatagcgaggtatgtatgtcacctctccgaattattttgactgtggttcctggactgtaaacaagctattatcttctggtgttgatgccagaaaaaacacaaaagtttgtcgttatctctactaacggatcataaaggggtttgtaactggagtttcaaacttaaggtatctaggcagtagttttgacattagatccaacattgtgtagtattcatttgtgtgtatcaatctatagggtttcattaaatttcatttgtgtactgtttaggtgttgaatatattgttttacttgttttttaactgaacaaaagatagctgaagctttgttctttaccaaatgcagtagtgatcatcacaatatatttttttacggaacaggagattgtataaaatggtttccatcggcggccaacggcgaccgctctgctctgacccaccacccaatccatccatccactcgccgccgcccctgatccaagcctccgccgcgcgacagcgacgcaccgccgtcgagaggaggaggcgtgagccccatggggaccctcctccggccgcgtaatgccgctgcacggtaaccacgcgcctctcgaggcctccgccgctagctgatctcttctcatcctgtttgggtttgggtttgtgatttgggtgttttttccgcagcggtggtggtggtggtggttgcggcgggagggggcggtggccgcggccgtggcgtggagtgccagctgcatcgggtgcaccgccgccggggtccgcaggttgtggtggcgacggcgagctgaggaggcggagggagactggtgagggacacaggcaggcaggctctcaaggctaagcttgttacaggtactgagactagttactaattactttgataatcagtataaataagcttgtgtagtgtaatggcattgtgcatttctgcacttgtaaattttacagaagatggtcattcaatttgaacctgcatctaatattttagtggtttgagtttattctcccagtcacagagttgaagaggcaagtaacctgtaagagaggactgaacattaacacctcttgttcgattaaaaatgaccaaagagcatcaaacatgtattcgaggctgttactttaatatggcccattaatttgtttagttggctatgtacatcctagttggtgcagtgttgtggaaaacggaatacgggtgtcggatggacgaggtgccgtcaagcgattaatcgtaatacggatgattaaacggaattatatggatttttggcgttcgcactaagatgtacataattgatgttaatggcaatggtggagacaaaatgcatcatcttaataaaaaatatttgtataaatctctaactatattatgaaaatgccatttattagttcaatagatatcaacactgatggttagtagcgcaatagcattgggcttgttagtcaaaatagtgcagctgggctgcaagttgcaagtttatgttagtttcataaacagacatctgatttgtcgataaataaccgactaatcgtgccatacaactgtataattactctgaaatagtaatgttgctccgacttgatgatacggtacggtctggctaccgtttccgttttgacagacgattaaacggctgtgccggtcgacttccacaacactgagttggtgtaaatgccagttaccatttctatgatctaaaataatcaactcttttagtatattttcaaaaacgaaaattcagtacacatgcatgaatcttaatcttcatatctagctcgttacaaaatcaacaaaggcaccgtgtcagctggtgcacattagctagttcgtacttagcattatccactagcaccttattttcatgcatatcatgctaatttgcttgcccacgttgagtgggaatttttttccatgttttataatttatatatgttctagacttctacttcatgttcctgagcctctagtatggctggtagcagactaggtgctgaatgctgtccttttttgcagactgaagagaggagaaatacaagactgtccgttgttagtcagatttgtaaaaatagacactgatgtagtttatttttgcccctattttatatttaacaatacaaatatataacgtatcctaagaatttatcgtaatttaggagaagttgctcgtttcattaaattaaattgggaagtaaaaatgtgtgctcgagtatgtcaatgcaatcctgtgttcttgtttgaagatatggtgtagggcaggccaggattgaacactgaatggtaagactgcttctgctttcagacgttattgctaaatttttagctagttgcaattagtgctgtcacgccgattaagcagtagaacaaagtaattttgtcgtgacaaatgagttatatttctttgaaaatcgaagcgaaaacgaaccaaaagatagaagaaaagggaaacttggtaattactccacaaagagaacaaatttattggtaagatttgatatgagatgctcgattacttggcttaagttaacaatatcaaatttggggaagcaccaaaagaattattgtgacttaagttaaagatatcaaatttggggaagcaccaaaggaattattgtgatggagttgtgggtgcataacgttatttgctttgttcaaatcctagtgactatgaatatgaatattaatgcgtaaggtaaggaatttattgttaattttaggttctttacgattgtgtccggggacgccattcggtaactgtaataatgttttgtattggattcacttgtgttacatgcacgcactaaacatgtgctttaccttttcatttgtttgtgcgttctgcgtttgaatttgacgagattccatggtcagctcaacatgtcagttactgcgtgtcaagcagttactgcgtgtcaagcgatcttatatggtatgcgcacaagcgattgtatacggatatgacagtataacgtgtgatattgatttttttatataaaaaaatacgatgttactttccttcataaaggaacaaagactttttttttaaaaaaaagaaggggtattactaaaaacaaaaatgtcaaaaacaaaatatcagtgcacatggcaagtgtgctcggcaattttttgtctgtactttaaacaaaaatatttctatatggtattttttacaagggtgtcacaaatattttaaattagccaaacatctgcattttattaaaaactgtataaattataatttatactctaaaaggttgtgtacatctctcttggagaaaatgtataagttgcgaacaaacattaatccacgttatataagtcaatctgttatttaaccatagaaagtaagaaacctactagcgtgttaagctaagctctctttcattctctttcttcttcctggttttgcttcaatcacttgtcaagtgaagggttcttaactaccattactcctactcaccaaatttttttctcagatctttcgtaggtatatattgatcctacatcttatgatcttaagatgatatccttctcattatcctctgctgaaactttagcttgaaccgtcatctacaccacaatttgagccccttagcacagagcacaacgagcaatagcttgcccttacgttcattatttagcatgcactactactaactacccaataatcaatacatcggttattaaactgtttgtacagtttaataatgtcattttatcacgttaacatatgtttcattcaacaccacaccggttttggcacagttgcaaacttgcaataacatttttactacttctccaccccataatataacaatctcgttccatactagattgctatattacgggacggatgaagtacttctttccttccaaaatataagaatatagtactagattagatattatttggattcacgaatttgattaggctatctagatttgtagtcgtacgtaatgtctaattcggtaataggttattacctctttggatggagggagtagtttttatttcgtactccctccgtttcatattataagttgttttgacttttttcttagtcaaattttattgagtttgactaaatttatagaaaaaaattagcaacatttaagcaccacattagtttcattaaatgtagcatggaatatatttttataatatgtttgtttttttattaaaatgctactatatttttctataaatgtagccaaatttaaagaagtttgattacgaaaaaaaatcaaaatgacatataatatgaaactgaggatgtagcagactatagcaaatttaaactatgcttttattttagagcatcaccaaaagattagcaataatttatccctaaaattcaagttttgggtttcttaaactgaaaataggaagtgaaaaatcttttccgtccaagagatagcctaaatcttatcttaactaattaaaatattcataattttcctttcgtcacattaaattttcgtccgtaaatccgattgaaatccaattggacaatccaaaaaatagagaaaaagaacagaaaaaataataaaaagcacacaaatcttatctcaatcccgcgggaagctgccgacgccgccgaatccgctcgagcgccgccgccgccgccgccgctcacggggaacgatgtcgctgctgtcgcacgcggtatgggagggcgccgccgccgctgcttgggagataggatatggagagagaaggaaatgtgagggagggttaggtttttccccatccgtatcttcagcgacacggaggcgatccaagctgtccatcagatcggacggctcagaacgcctccatcgtcaggccgcgcatgcttgatgggccgagggaaggccggagggtcgaacaaacgcagtcagaggaggagttggaggaggtaaagtagaatttatttgcgggctgagatagtaaatggactgaaaatggcccatagagaaattgggaattttatttaaataaatgttgaaaaggtgtttatattatcaaaattagaaattaagctccgaaaattttaaaaaatattcaaagagcattattaatcatgattaatttaataaaaattaaatccaaccatatcatattatttcacggcgcacggtaggaaaatgcgcagctgttgtcgctgacggtgggagagaagggacattgtttatttccagaactatcttttataactcccatggaactttaaaataaatataatcattattatagcattagtttttttctgtcttttttttccccaagagcgccgcgcagaagagatcgatcgcgatctccctgccccgacgtcgccggccgatctctcattctctccacgccctgctcgtcgccgatctcctacaccatccctgccatctcctccttcccctcccctctatcctccactggtgccgcccacctctccgtataagacaaactgcgttgcggcgttggtttccgccggcgctgctgctgcacctgtcagctagggcgggcatggcgcgccgcgccgcttcccgcgctgttggcgcccttcgctcggacggctcgatccaagggcgaggaggccgcgcggggggcagtggcgccgaggacgcacgccacgtgttcgacgaattgctccgccgtggcaggggcgcctcgatctacggcttgaaccgcgccctcgccgacgtcgcgcgtgacagccccgcggccgccgtgtcccgctacaaccgcatggcccgagccggcgccgacgaggtaactcccgacttgtgcacctacggcattctcatcggttgctgctgccgcgcgggccgcttggacctcggtttcgcggccttgggcaatgtcattaagaagggatttagagtggacgccatcgccttcactcctctgctcaagggcctctgtgccgacaagaggacgagcgacgcaatggacatagtgctccgcagaatgaccgagctcggctgcataccaaatgtcttctcctacaatattcttctcaaggggctgtgtgatgagaacagaagccaagaagctctcgagctgctgcacatgatggctgatgatcgaggaggaggtagcccacctgatgtggtgtcgtataccactgtcatcaatggcttcttcaaagagggggattcagacaaagcttacagtacataccatgaaatgctggaccgggggattttacctgatgttgtgacctacaactctattattgctgcgttatgcaaggctcaagctatggacaaagccatggaggtacttaacaccatggttaagaatggtgtcatgcctgattgcatgacatataatagtattctgcatggatattgctcttcagggcagccgaaagaggctattggatttctcaaaaagatgcgcagtgatggtgtcgaaccagatgttgttacttatagcttgctcatggattatctttgcaagaacggaagatgcatggaagctagaaagattttcgattctatgaccaagaggggcctaaagcctgaaattactacctatggtaccctgcttcaggggtatgctaccaaaggagcccttgttgagatgcatggtctcttggatttgatggtacgaaacggtatccaccctgatcattatgttttcagcattctaatatgtgcatacgctaaacaagggaaagtagatcaggcaatgcttgtgttcagcaaaatgaggcagcaaggattgaatccgaatgcagtgacgtatggagcagttataggcatactttgcaagtcaggcagagtagaagatgctatgctttattttgagcagatgatcgatgaaggactaagccctggcaacattgtttataactccctaattcatggtttgtgcacctgtaacaaatgggagagggctgaagagttaattcttgaaatgttggatcgaggcatctgtctgaacactattttctttaattcaataattgacagtcattgcaaagaagggagggttatagaatctgaaaaactctttgagctgatggtacgtattggtgtgaagcccaatgtcattacctacaatactcttatcaatggatattgcttggcaggtaagatggatgaagcaatgaagttactttctggcatggtctcagttgggttgaaacctaatactgttacttatagcactttgattaatggctactgcaaaattagtaggatggaagacgcgttagttctttttaaggagatggagagcagtggtgttagtcctgatattattacgtataacataattctgcaaggtttatttcaaaccagaagaactgctgctgcaaaagaactctatgttaggattaccgaaagtggaacgcagattgaacttagcacatacaacataatccttcatggactttgcaaaaacaaactcactgatgatgcacttcagatgtttcagaacctatgtttgatggatttgaagcttgaggctaggactttcaacattatgattgatgcattgcttaaagttggcagaaatgatgaagccaaggatttgtttgttgctttctcgtctaacggtttagtgccgaattattggacgtacaggttgatggctgaaaatattataggacaggggttgctagaagaattggatcaactctttctttcaatggaggacaatggctgtactgttgactctggcatgctaaatttcattgttagggaactgttgcagagaggtgagataaccagggctggcacttacctttccatgattgatgagaagcacttttccctcgaagcatccactgcttccttgtttatagatcttttgtctgggggaaaatatcaagaatattataggtttctccctgaaaaatacaagtcctttatagaatctttgagctgctgaagcattttgcagctttgaaattctgtgttggaattcttttctcctacagtcctattagaggagggatcttctctgtatgtgtaaatagcgaggtatgtatgccacctctccgaattatttttactgtggttcctagactgtaaacaagcaattatgttatgctgttgatgccagaaaaaacataaaagtttgtcgttatctctactaacggatcataaagggatttgtgactggagtttcaaacttaatgtgtctaggcagtaattttgacattagatccaaaacaatttatagggtttcattaaatttcatctatgtgtactgtttaggtgttgaatagtttgacttgttttttaactgaacaaaagatatgtctgaagctttgttctttaccaaatgcagtactgatcatcacaatatattttttatggaacaagattggattgtatagaatggtttctgatctgattatcttatctcaacgtattattatgcacatgtactaatcatgaaatatctgatggaatgatgtttctatttacctgtgtgaggcagcaaggagtgagatggataacaccacatactccctctgtcccagaatataagaagttttagagttggacacgattattaagaaagtaggtagaagtgagtagtggagggttgtgattgcatgagtagtggaggtaggtgggaaaagtgaatggtggagggttgtgattggttgggaagagaatgttggtagagaagttgttatattttggggagtacattattattctagaacaatactgttgtgctcaagaagcgttccaaagatgtttcacaacctgtgctcgatgggttttgagcttaatcctgggacattcagtatcatgatctgtctcattcttaaacatggaataaaggatgacagcatgatttctttgtctctataatcttttggctacccacagataatagctgtaaatctatactactttaaaaggagtagtggtggtggtgagtggtgaatctgccaccaccccaccaccaactctcaaaattctgacatgtgggatcactgtcaatcccttctccaagacatgtgggatcactgtcaatcccttctccaaaccaattgtatgatagaacagtggaaatcacggacagaccatggagctctcaaccataatcatccttgcgagttaataacaaatggagcgtaaacttggcaagcaaaaaactcaaattaattctaaaattaagctctaggattcaaaatagatttcctctctgcattgtgctgttatgatttttaattccgtaacaacgcaaatgcattttgctagtcttataaagaagggttaatgcaaatattctgattaaatgattgtatctatgaagtttgaatgctagtggaagctcctttgaccatgttttgttgtgcgagcatttaagagagtgaagagaatgcttctttggtgctgttctggtatggaaggatccacagataaaattcaggttctactgcttctctgcttgtaattttcatgaagctgcagtgaataccttgttgaccacttgatctgttgctttgaaggagaatatagtagtggccaaggttggtgacggtgatggtggcatgtgatcccccagatcttcagtgacccagagaggaggggacggcgcgtggtgagctacaaggcatactcagtggagggcaagatcaaggcctcccgtccgtaggggactccgctgcatcaaggccaactgctccgaactgatcaatttctggtacggatcacttctcctttccttttttttttcaccttaagcactctcttgattcttcgctgctacctcccttaatttctttcaatatattgtggcacttgatcatggcggagacccaccttccagtgtgaatggattttgtcaaagaactaaatttattccattagcttattttccgattacatggaagacattcttttctggaataaatacagaactaaatcctgtttcctgaataaaagttgttagtgtgtggcatggtgcatttccgcgcttctaaattttataaaacctgttcattcaatttgaacctgcatccaatccaatattttaggtgcagacaggtgcttgcggtcaggttaaagaagttggcaaaaatgcttctgaagaaaggttaattgttgtttcatctcaggaggtaatatgcagatgattattccaattggcattgccttgccatttttatcacgagtctttacaattttatatcctcctacatattctttccagattccagatgatccagtgtctccaacaattgaggcgcttattttgctccatagtaaagtaagtacacttgctgagaaccaccagttgacaacacggcttgttgtaccatcaaacaaagttggttgtattcttggggaaggtggaaaggtaattactgaaatgagaagacggactggggctgaaatccgagtctactcaaaagcagataaacctaagtacctgtcttttgatgaggagcttgtgcaggtaatttatttggccatacctacaccagagatccatatattacttttataactgcagtttttacttgttaacatttcattgtgcttttacatttgttccaagctttcaggttgctgggcttccagctattgaaagaggagccctgacagagattgcttcgaggctttgaactaggacactcagagatggaagttcttccaataatccgacaccttttgcccctgttgatggtcctcctgttgatatcttgcctaacaaggaattcatgctatatggacgatctgctaatagtcccccatatggagggcctgctaatgatccaccatatggaagacctgccattgatccaccatatggaagaccaatatccacaatatggaagacctgccaatgatccaccatatagaagacctgtcaatgatacatcatattgagggttgaacaatgatgggcctcgtgatcaggcccggtcctgaggggggtcgaatggggcgatcgctccgggccccccgattcccagggcccccacctatctgtgcaacgagtagtagcgatcttccagcgcgcaacgtgaggcgatgtttctccgtgatttcgccggcctgcaactgcgagatcgcgagtataacgatcagccgatcgatctcatctgccgactgccatgctgatgccacacgcaagcgcagcatatcagccttatcttggttgatcggcatgctggacgagcacatctgttgtcgcatcaactgctgactgctatatatgtgctggtgctgaatcgatcgattgtcgtcacggaagtgaagaacaaccacggcactgctgcctgctgggctctagccgccatcagtaagtacgctatactgcctatctagatctagatcgagattacatagtggaattatctgtttataacaaaattacaaggtatcaattgataatttaaggttataaccgtacaaacttcagtgatttgctggtttcacattggttagatttgtttcaactaatttggtacttctgtagccttgtaatttacgaatctagtattaatattttcttaagtattagcctgttccttgatattatgctgttgagaaagtatgcaatagataacaaaaacaagtaggtgtgttgaggatgctcaagagtaatacaggcacttcaataattctgatattatcaggacatcatcaataattctgcgcctacaaatcttcaaagaaaattttaatataatgcgtatgattttttaaatacgaatattgattgctatttaaagatatttatattatatggtaattattatttgaaggtttataataaaggcctccgtttttagtttcacgctgggccttcagaatctcaggaccggccctgctcatgatccttacaccgtgtatcctgtagagtacttctctaaaagagagtaccctagtggaagtagcaaagttgcaccatctgcttcatacgaaagatatgcagcaactactcgcttgcctaatagagaactgccctcatctattagtcctggtgccgattatatgtcctgccgttcttatcttgaccaagtacctactgataggtactctaatagggttacactacaattaggcctcttgagagccgggaatagtaatgtgcaacaattaggaatcaccagagctggaaattccaatgcttatgattatactgaggtacatttccaatgcgttagcttgcctcttctttgcaaatggccctcgcctgatatgtttccattagaaacatgaaaccatatatttgactgttgcattatgtctattttcttccatgatggttcagacgtctgaaaaaaggacaaaaatattctagaatatgtcatggtgatccaaatatatccttctgtcttgtgcccactctaatatctatcgttggtaacactattcaattgttaccatgttgttgcaaaccctagattcagttattcagctgttctctgctgctgttgcttaccagttttcttagttgggtgttgatcttttctcattttttatttccttgtttcctggttcacctgctgcctctctgatgcatctgaatgtatatttttgttctcttcagtgcttaatagatttaaatttcattcttttcaggctgcggagctgatccatggacgtgaggattaccgaagactgtcaggtctcactgggtatggcttacgcagactgaatttttacaggacacaaacatgaattttgtcctcataatcattgagtgatgatctctttgcaggtatccaggtggctctgtcgaattgtggattccaaatagttaactggagtctgtcattggtgttggtggtgtcaatctagctgagatccgtctggtatagcgtaagagaaacatcatgcactatccccagtcataaccatgccccaatggccaccaatagttttcctcgtgaaaatctccccttgatcccagatctctggtgcgagagtgaagttgcacgaagcccatcctggttcttccgagtccattgtggagatccagggcattccggatcaagtgaaagccgcacagagccttctgcaaggcttcatcggcgcaagcagcaacagcaggcaggcgccccagtcctctcgcatggcccattatttttagtaagctggaggacattcgcaacaggggggtcagtggtcactgcaaagctgagtttgttcttcagttcaactgcagaaaattgcagatcggttgccgtagttgctagaacggtacatagttgccacctaactgtagcgagtggcataacttattgtgtgttactgcccaatgttgtctctccttgtgttcatggattcagacttgtgattgtagtatttctggatcagactggagtaaaagaaaaaaaaaaaggaagacatgggtttaacagtaagctcaaaacgttgacagtagtaaaataaaaggggtttgttcactttatttccaatatcaaccttaccaacatttggcgttgaatcatttataccacatcgcttgtgcagctgaatttggggctgtttaaaagatggtctcttggattgctaattgcctcgcggcaagcgtggtaccttgtacaatataaatataattataactatttaatttcataattaaacatgttgttacaaatctctactattataaaaattgaagatgttttttgccggtattttggtacgtcatctgtgtatgaatccgtttttaagttcgtttgcttttggaaatacatatctgtatttgattcagtttataagatcgttcacttttggtaatacagaaggaatcatataagaattctgtttaaaaacactcgtatagtaacttgagacgatcagacgcctaactacagctcatgattttctaaatatatatatatatatatatatatatactagaaaaaatatatgtgtgttaaaagctatcttaatcttattattgttatatattttagttaacaagaaatctattgtgggaacttgtttggatatatatttttttaaaaaaaatcatgagctgcaattaggaatccaatcgtctcaagttagcaggagggcgagtttttttaaagagatttcttatacgatttcttctatatttctaaaagcaaacgaacttaaaaaccgactcaaacatggatctgtatttccaaaaacgaataaacttaaaaaccgactcatgcacagatgattaatttttataatagtagagataaacgaactcccacagtgaattttattttaactgaaccatataacaataataagattaaaatagacttcacccgttgcaatgcacgggcattttttctagttaaagaagaaataaaaaaacacaaaaatttataaaatgtaaaaaagaaaaatattataattttgttagaattattattataatatagaaaaatagttgccaaaatttctcaacgaatgtcgaataaactcagcaatgtcatatatttaaatatgatggtaatatttgttcgcaaaactttaatcttcaatccttcaacaacatagatatacaacgtcgtaatcgccaacaagcccgagtgaccatacaggatagccgagcggtggatctgtactgttcttgggtgaaataaatctagtacattgtatatcttatcttaatatctactattataaaaattgaagatatttcttcaaagatttccatacgttctctactccgttacaatatcggttctactccgttacaatatcggttttgtacaccccgcgcacgcgttgtgtgttctcccgttccaatacatgaagctagagtcttgcttctccctggtctggcaggccctttttccaccatccccaccagggccagcgggttacattgaccgatcacggcccacattagtggatgcagccagccacgctcttcacaaatcatgtgatgaacattagctgagttaaaatttatcctttgatgattgttagaaatgtttttttctccacatcttctctttcaattttggaaaaatagatttcttgatttttgtgctcgtacatcactaataaatcagttgttacccttccacacattgtcaatttaccatgtctatttcagctcttaccttgtatagtcttgactcttgagtcctcgctattgactaagttgctacatgcctcctacaaatcaatagactgccataacaatattttctacgacatgatccatattagtccatgcaatgcaagtacacacacactactgcacgaaaaaactatgcaccataacttcaaaactaacatgttagaatgacgttaatttttcattacaattatattcatcgaccgttaatttactaggcatcctgtttaaaaaaaatattcaccgaccatacccacatgttccgtagttcattaggtgatggatcggtagttacagcagctggatttttatattttggtcattttgaaaaatttatttcgcaaatagactcctgaaaaaacttatcccagaaatagtcccttttggagcgtcagagtggctggcgccgtggtccaacgggacagcgccaacctctctggcgccgccccccgcctctattcttgtttctctatatagagttgcaaactttttatttttgttttatttttttggatgttttttcactcttagaatcacgatacaaccaactacaaaaaaaattaaactcgaacggaatatatcacttagctagaagtctgaaaatatagcataccacttatctactttgcaccttcaccaaaattagaccataacttctttagtaaaatcctttgatcagcatattaaacataatgcactctatcactaggtgaaattacttaatctaattcaaaatataactacatgtagccttgaaaaattctacatgccacatatttcgtccgtttgagtttattatttttatggttcgttcatgtgagttcccaagtgtgaaaaaaaaataaaataaaaataaaaaagttgcacatcctctcctctgcattagagaggagaggagaggaaaaattctacaggtcacatatttcgtccatttgagttcattttttctatggttggttcttgtgtgttcctaagcgtgaaaaaaatatcaaaaaaataataataaataaaaaaattcggggggggggggcgccagccactcttaggggtgaaaacgatcggataatatccgatccaatctgctccgaatccatccgaaataaggatatggtatgggtttttagaaatctggcggatatggatgcggatgaggatatggtatctccgaaatacgacggattatccgacatttttgtcggattatccgataggccctttaccggataatccgaaattatgaacacatgtaaccactctatctattgcatataacataagttggtccatccaatgacctaattcatcaattaccctagatttcttactatgtggttttcaccatttcatgtcacacttgcgtagctgtatttttataaaatggacatcatgtatttatgttgtttagcacttaagcacataattattacaatgggtcgtttattgacattgtgttatttttacttgcattgctaactcaatgttgtattgattgcatacacacgtaacatctgataaaatttaatccgtttctgaaccgattccgcaccatttccgacatctgcatccgtacactatccacacccactccgaatccgcttaaaaatatggtttaggatatggtatgaccactatccgtccgaatccgctttattttcacccctagccactctggcgcgcttcccctgccacctcagcatcgtcccaccacgtcggcagaaggacggcggctccagccactctggcgccacaaaaaaggaccatttctagcataagtttttttaggggtctatttacgaaataagtttttaaaaggaccaaaatgtgaaaaatccaggttacagcagactgtgataagcaatagctatattgcctatatatacacgtatatgcattgctaatccttcaattttgtccaattcttttaaattgtcttcacctgttgcaacgcatgattttttttctagtcttaaccttaactaatcttaataactaactaaaagattcgtatctttccgatcgtcaccttgtccatacgctaatttttcgtccgtcccccctccccctcaaaaaaaaagggaaaaatccattttacaccctcgaactcttatgcttgtctaaaatacacccccgaactataaaaccgggtataatacaccctcgagctatcaataccggacagttcaagggtgtattatacctggttttgtagtttgggggtgtattttagataagcataagagttcaagggcgtaaatggacttttccccaaaaaaaatcccagtcgttactttccatcctgagaatcggagacagggaaaactgaagcatacacgcaaatagaatcaaagatagggaaaactaagcatatacacacaaatatatccaaaaattcccatgcagctagatcgggtgccaccgttgttgccaaaccaccacattgcaatgtaaatctaagactaaagcctaaatcctatgctaagtcatcaaattagactcggttctaccaatttggtaatatatcaaattagacttgatttttactgatttgaggttctcgaggtgtcacactatgaaacggaagtttttcccgttgcaacgcacgggcactatgcaatatcttaactaattaaaagattcatatttttcctttcgtcacaccgatctttcgtccgtctgtaacatcacgtgcacctcctctccaaatcccacatcatcataatccgacccaaaaacaaaatctcaatctcaatccaatcagaatcatcacaaaatcatccaaaatatcaagagatgattataggagatggaggggtgagcaggagcaacatcatcatcgcataaaaaccccaaaatcaatcacaacaacgacatcattatcacataagaaaaacaatacaaacaacatacacaatcaacaacactggcggatccagccgaggggacaacggcgtggcagcgggcagatcctctcggtcagatccgcccacgggtgccactgacgtcgccgccgccaccggatccaagggagaagcttcggacagagggagaggggggtagaggaccgctaaatccgcccaccggaaatgccgccgccaccacctccgtcggatttgcccgagggagcgccgatgccgccaccgccatcgcgggagaagcttgggcacggagggtgaggaggagggggggtagagaatcgccggatccatccgctggaaaagcctccgccggatccgcctgccggaaacaccggtgtcgccgcctccgccggattcggtagcgggagccgccgatgccaccaccgccgccggatccggtcggtgggagccactgacaccatcgccgccgcctcctctgctaccgacaagggagagacgagaggggcgggggcgagggcgggggacgagagggttagagggagggaccgagtgggagagagagggacgagtgagaggagggggacgagtgaataaggatgcgtgaccttatccactcgcgcggtcgcaccccggctctttctctcgctcagctgttgcgcttgtggagaggatgcgagatttttttttgagtaaaatgcacgggcggtccttaaacttgtagcggtctgtcatctaggttcccaaactctcaaaatgcatatccaggtcctagaatttgtcaaagtgtatcatctagatcccaaaccgacacatcctctcttggatcctacatggcgctaatgtgacttgtcacatggacgtgacacgtctttttttttcttcttttctttttcttttccgttttcttctcattcttctttttttccatcttctgctcgggtcacatagaaaggaaaagaaaggaaaatacaagagaagaaaaaaagaaaaaagaaaatttttaaatgggtctcattcgtcagtcaaaattatgccacatcatgtccctgcgacatgccacatcagcaccacgtagcatcctgaaggggttgtggcgatttgggacctaaatgacacactatgacaagttctaggacttggatatgtattttgagagtttaaggatttatatgacacactactataagtttaaggaccgcccatgccctttactttttttttttacacggagagaatgcgaatttgttggttagttgcggctgagggtttctcgcacggagaaatttgcggtgggagaattttttttcgaggttctttctattgggagaagacgggattatagggattattactggtgtggtggcccctgttttctttctttttcgagcttctttccgttaaattcacttttctctcttcaaggagcgtaggacatgactgaatgcagctgctgtaaattagaaataaaaaagaaacatattctgtttttcatttttttcaataggtaaatataaagatttttaagtaatatttaaaaatatatagtgctgatcaacgacattgttaagtgagattttgctgttactatcacttttttttccattgggctcacgtacggcattaaaagttttagttttggttctctccttttgagtttgggcatataccaatattgagataggtatactaaagttcatttggattttattcgattcaacttttttgggttttgttcagttcttttttacatgtttctcatctgaaattaggaaattaggtttggtaaagtcttgaatagataacgctgttgacgtttgaacatatatttatctatttatttatttaaaaatatatgaataatttttattttgttatgacttttgtcggtgacatgggaccgggagtatcatgactagaggcttgggcaggagcgatcacccacgtggcctgatgtaacatcctgaaaattcccaacaataaaaatcactaaaattttgaactttttaaaacttttgcatcatgctggttgttatgattgctattgcttgccaaaccgtaaatgatcacaaagaaagtaaagtaaggatctaaaatttaagtaatagataaatttacgagaatataatatttaattgctaaccctacaaataattacgcacaagaaaacaaagccagacaaacggaaggttaattactaatttaaattatggattaattattaaatacttgaaccatgtgttgcgtgccatggcatctaaatacacatgaaataatggtcatataattaaattaagctttataaaattatgtgaggttttaattaagcaattagcttaatgttgtaccgagtcttaatatactatttatagaataaataaattcaacctatccgtgtaaaatatattgctataagttcattcaatgtactattgtaataataatggccacattaggatattttaattaattttggaaccctcaaagcctccaaaattatctaggttaattttgaaattatacctcatttaagtaatgcaatagaaaaatatacataaaaataaaatatgggtaatattagaaattgagtaaattttcatctaaattaaaacatatattgggtaaacctcctttatgtaaaaattaagatttatagaatgaaatttgtacaagggataaactaaaatcgggttaaatagaaaatggcactgttcattgcactctaggtgctcgacgtggtccctggccctattttccccctcagccgcgcgcgcctggctgcctcgcgccccgcgccacgccacccgcgtcgcgtcgccgctgccgcgccgtcgccgtcggccgttccgcgccgctcgtccgtcgctccgccgcctcgcgccccgcgccgcgtcgtcatcgcgtcgccgtcgccatcaccgcgcctggccgcccctgaccccgcgccgcgccgcgccgtcccgtagccgcgtgcgcgttccatcgccgctgccgcgccgcgcgccgtcaccgcgcgccgctcgtccgccgcgcatagccccgcgccgccgcgccatcgtgtcgccgcgccgtcgcgtcgctctcgagccccgcatccctctcgagccccgcacgtcgcgtcttgtcgccgttgctgccgcgtcgtcgtcgccgatgctgtcgcgtcgccgctgccgcccgtcgcgtcgccttgcgccccgtgccgccgctgccgcgttgtcgctgtcaccttcgcgtcccgcctcgtgccgcgcgccaccgctgccgccccgtcatcgcccgctcgtcgcgcgcgccgccgccgctgccgcgccgtcaccgtcgtgtcgccgtcggcctcgcgccttgagccgccgcgcgcccgtcccctcgcgcctgcgccccgccgcacggccgtcccctcgccgtcgccctgcgccactgccgcgccgcccgtcccatcgcgccgagccccgtgccgccgcgcgcgtcgcgtcgccccgcctgtcacgccgctcgccgcctcgagccacacgcgtcgcgccgtcgcgtcgccattagggccggccacccctttccccgcgccctataaaaccccccggccacccccctttcaccccacaccatccccacccattcccctcttcctctcctccttcccctcttcgtcccctccaccgcgccgcgccgccgccttcgtgccgccgcgccgtgcgccgtcgtcgcgccgccctcgcgccgccgcaccgccgccttcgtgccgccgcgccgtgcgccgacgtcgtgccgccgtcgccgtcgccgtcgtcgtgccgccgtcgccgtcgccgtcgtcggtaagccgccgtcccttccctcgttccgacgccgtcgccgcccgggtgggaaggagccgagagagagagggaggaaggagccgggagtaggaagaaagaaaagaaaagagagagagagaaaagaaaagagaagaaaagagaaaagagagaaaagaaaagaaaagagattagagaagggagggaagagtgggccccacctgtcattagccccatccaattccccttagaaaaataattctgtagaaaagaaaatcaagatcttgaccccacctgtcagtcactatagcgtgtggataaggttgtattaaaaataaatgaattaggaacagtactatttcgcaactattagaattaattcaaatttgaatctttacactagcataactaattcattttagctccgatttgagtggaacttgaacctaaattcatctaaattcataagctttccaatggtatataatttactattaaataaaatatatttataattattaagtaattaatatcatatgattaggttatggtcaacttaaaaatatgctaataaataaaattagtattgtggatgtaataatatttgtctctaacatgtcttgccactgtaacaaccacacaaactaatattaagtgatgtctgaaatgaatgaatgaataggaaaatactagtacttgtttaatattcgatagccatataattaaacccatggcttataggttatttaaatcaaatgtagccttgtgattatgcaactaaaatataaacacatatagatgaatctttagcttgattaggaggaataataacagagctagtgtgactagttatgatatagcttgttgtcggttgcctatatttagtaaatggttcaatgttaatacactgatgcacacacataccctttttgataacctactagttgcatatattaaacttggtaataaatgaagaaccaatatattagctaaatactggtgctagttataaatcttgaccacacataattttagttcaaaccacacctgaggattgttcgttataaagttataaagttataaagttatacaaaagataatatgtaactataatagtattaaaccacaaatctaaaatacagggcgcataattgtcaaccttttatgcaaacggataatatccatatatatacatcatgtggataattcgaataatagctccattggtaaaataataatgtaggcgaatcatggtgatgagatggtttatcctaaacctccccatcgacatagccatgctatagggacctgaccattttaccttcataacagatctcttccataagccaatagctagactaaaccacagattagcaaatgtgtacatcatatattgtgctagttagtaccaatagaaccatcaggacaatataaatactaaggaatcttagctcttagcttgattagaatccaatagcaaacacgagtagtatgagcagccttaggttcgacctcaataattatattttgcttgtgcataattgcttcttgttgaatattggtttttctcgcatattatagaaattgtatatcggttagtcgtgaggcaacgtatgcagctttcaggaggtgaaggttgatcaagattgtatcaagaataatgactattctaagcaggcaagtcatcactattccttgaacatgttgatcctaattgcgaaattattttgtttacaaataaaattgcatgcaatgatgaacatcctacttgtgattatgccatgccttgattattgtttacccttaaaatccttgtaaccatgattacgtatgagtccctagtcaattatgacaattgcttagagatgctattctagaatcatgcatactcatatttatcaaatgctatatgcttgggcaattacctttgggaaggtaattgagatgcggcatgtggagacatgaacgccacattgccatgatattaatgacatgatttgtgaaaggagaaataaaattaaacaactgttttcgactggggcggacggaggatttgggtggtatctggaaaaggctagtaccgtccccggtcaattaaggaccgagccatgaagttaagcatgaaacgacccccgtacaaccgcacttctcgtatgggtatagacctagcggagtagatagctgagcggaggcagtatccatgcatagtggtttcttgatgtgtgaggcaggggctctacggtggggcagccattggtaggaccgcaaggcgggtatctacagtggtgtcgccatcggtaggactgccatgtgagaatctaaaacataattataacttaatgcatgtgtgagtcttcccttcccgggtgcgccagaactcctctcactgctagaaaccgtgtacgcctagagtgcatgaggatgaaaagttcatggagcgggtactgccaatgcgaggttatcgaaaagctctgccgtgacgcatctcatgtgttgggacgaggctcatgtgttgggcagtcgcggagtgcgggtaaagtgtacatccactgcagtgtgagtaaaccaaatctattcgaatagccgtgctcgcggttattgagcaccgggacatgtattacacttggctagactctaaattcttaacttgtggggaatgggatattgcatgatgaattttatgctgatggagccacatcccgagaggagggaaggtggacatcctcagaaaaccatgacgattcaatggcgggaagctatccttgggatcacaatggatggtggacagaaccgtcgttgtttaaagtgaacactggtactaaaatttgatcgatctatgctaggttttaggcttgtgaaaagaattgtaaaattagctttatgcaaaaggacctgaagccattccttgaaataccctctatcatatgcattgttattatggtggcttgctgagtacggttggtactcacccttgctatttatatatcttttaggagagtgttgaagagaagcccttgtcggtacgcttgcgtatcccacaagatgatcggagtgcggtcttgttctaggtctcgtttccccagtcgactgcctgtggcatgttaaccgggcccttatattattttgtctttcgctgttgttctctgatagttgttggcctacctggccctaatgtaagtatttaactcttttagcctaaattcattcgtgatatgttgtgatccaactatgtatgtgtgtaccaactactgatccagggattggtacggataaacacagaagatttccgatttccaaaatcgggggtctacacctgaccccctcagggggggggggtcgggcccgagggtgatgtggccgcccccctctttgtctccccgaggggtcggaccgctcccgtttctgccccgagggctgaggcgccccgaccccttgtgggttttgcgccgcgtgtatgggttaggtgagcacaacggggctcacctaaccgtatttattgtggtttggacgagcgcgtcacgccgcatgtagcgcagtgcagcgcgctcgtttatccggtctgtgaccagtcacagaccggtcagatcgtgggttaggtggcaacaggcggtctgacacacgcctcgccccatcccgtcaggataagagcctccaggcacttgtccctagcccggagccagcatgctaactcctggagatgacacgttggtcccggtcagatatatgccaggcttcatcccaaccattacaagcaagatattgtatgaagaagggcgaacatgcagattgctggactgacacgtggtggacaagaatgaccgatttgtgaccggtctgacactggtcatgtcgtcggcagacaaccatgttcccacgttgcacctgctttcggcggagtggaggtaggtatgggccatcccatcagaaggtcgttcggacagcagccattgcaagtctccgcccatttatgaagagatgacagggtgatcccctggagagaaaaaaaggaggaccttgcccacttaggaggtgaggacgactggaaggggagaggatctggagagtagatcccacgagaggaaaaaagggagaagagggtttctagagtaagagctctctgactctccagctctttgtagcttcttcgtacacagatccaccagaaaataggagtagggtattacgcttctcagcggcccgaacctgtatacatcgcccgtgtcttgtgcttttttcattctcgcgaactttccacagactaggagcttagaatctcgcccagggcccccggccgaaccggcaaaggggggcctgcgcggtctcccggtgaggagccccacgctccgtcaactttggcttataattaaaaatactctaaggatatttttttatattttattttcttatgtctatatgaaattttaaataagatagatggttaaacatatattggaaaaacatatatccaaaagtccactatcacaagcgtagcatagatacgattacaatacgtttccgcgaagactgtttatacctactctattccctgttccttgtgcggttgtgccatttggggctgttttttcatctcggattaactcgcgtggaaaccgcgagacgaatgttttgagcctaattaatccgtcattagcatatatgggttattatagcacttatggctaatcatggcctaattagacttaaaagattcgtctcatgatttacatgcaaactatgcaattagtttttctttttatctatatttaatgcttcatatatgtgtccaaagatttgatgcgatgttctgggaaaatctttttttaactaaacatgcccaaggtgtttctccaattaagttgacccaaaatcattcggcgtcacctttgtctttcactttccttccactacaaggtgatgacactgacaaaaggtccaaaagctacaggatctgatttttgttcatccatctgtgatgtgtcggcaagccatccatggagttcatccactcaactcctctctctcagagagagagagagagagagagacagacagacacatgcatgatagattgtgctagtacggtagtaacattttattgcctccttttctaaaattctaggttgtttggaaaacaaaaattctagattgttcaataaattaataatattaggtatttattttaagtcactttaggtgttaatttttgaattttaaactgcttaaactctctttcgacgcatctgagagcaggtacaatagcagactataagccagctataaatatattttaagtagataaaagaggaaaaataagagtagcgggctatagatttgtagacagctgcagcgcgagctccaagatacatatgtgtatgacatgtgagaccaaacattaattatgtagtatatgtttatatgtatctattgtatgaattggctattaaattgactatgggtgtgttcggaggtgggtgttgggaaccatctcccaagcacggaaaacggagcggtccattatggcgtgattaattaagtattagctattttttaaaaaaataaatcaatatgatttttttaaacaacttttgtatagaaactttttgcaaaaactcaccgtttagtagtttgaaaagcgtgcgcgcggaatatgagggagaggggttgggaacctcctcatccgaacgcagcctatacatgatttggagccaatagttggctataatattaaacttgctctgagtggctcttgaatcatcgaagtgatagaaatcatatgcagaaatgtttatatttgtgatgtaaaatttgaatctaaaattatttatattttgaaatggaggaagtactacctaaaacaagtatgagaaagagacatgaaaaacacaaaatctagacttaaaaataattggaattactagcaggaggtcgaagtcaatcaagacggcgaagaaaagcacaggggacagcagacacgttaacacgtaagtaaacaaacaagtggttaattaattagggggccctcaagtctcccctaaagccactaaacatgacaggtttgtgtaccatggaaaaaagggtgaagcaaaactttattctctctctcattagattaccagttggaaagcaatcctgggacctctagctaatctcattattgtagaacaacgttttcttagagagagagagagagagaaataagtcaataaaaattactactaatccacttgaaccagttctgtcggtgtcggatgatttaccacatttgacgaaacggactatttattcgacgtttcgaaaaacacacttttttagaaaaaaaaaactttcctctattagccactcgttttagttatatacctatccgagtatctgttaagtttatttatcaaaatatttaatttatctctataattaaatatacaatccgtaaaaacaatcacgcagtaattcgtttcaaactgagcctcagctagaaaatcaaaatggaaatgaataacaatagcaacagtagagttagtttttcggcttatcatccgcaacccaaatgcgaattttaaacttagccttagagttaatttttaaggcttgtttaccatacttcattttcccagcattagtttcttttgtcactaaaaattgtttttttaagttgtttcgttcattttctcacggtttatcagcagtagagcgaagccattcttggagcctgtttggcacagctctagctccagctctagctccactctttctggagctggagctcagcccaacagttttaggtgcaccaaaattaggagtgtagttgggtggaactctctcacaaaaaattgtggagctggatttagacagctccacaacttcactccaaacccaactcctgaagttaaattgataagttgaagctctatctatcaagccctttttcttgatcatgcttctacctactccatttttgtttcttggccctcacaggaattggaaaggaaaggcgtatatgcatcaatgcatgcatgcgcacatcaacctcgtccatcaaccatcataatcatcatcatctcgccagctgacgaaaatgacctgcatccatccatcacggacaatccaagcgaacaccgctaccaacatcacagccaacctgtttatcactagctcttgataccactcctacataaacactacgcgcaggttaattaattaagcgtgattactgaagtaacatctaatcacgtcctggttagcctttaataagacaacagttagagcaggtacaatagcagcaggatataagccagctataaaaaaagagagaaaagagcaacgggctacagatctatagccagctgtagcatggacttcaagacacaacgtgtgtataacaggtgggaccagataataatagtgtagtatagtaagtaactattatatatattgactatagatgatttggagctattagtgtgctatagtattaaacttgctcatagagcaggtacaatagtaggatattagccagctataaacatattataatgagataaacattgatagagaagagcagcgggctacagatctgtagccagctacaacacggactccaagacacaacgagtgtatgacagatgggaccagatattagtagtatagtaagcaactattatataaattaactattacattggctatagatgatttggagttagtagtgggctatactattaaactttttctcttagcaaaaatcaagcgcctaatcacattagaggagtagctttgagacaaaccaattagcggcgaatcaagcgatctgcgtggtcgtacagtgatgggccgggccgggcccacagcccgacagtgacagggggcctgacgcatgtcagcctcagccctggacgggagctagccgttgtgtccccgggggaggggaggggggcattcccatcatttcgcccctcctccgggcccacatctcagtgggggtaaaggtgtaaattactgcgaccgcgagtccagcgagcctagatttggaccttgtgtccgtttgactgaaccggagctactccccaatacggggggattgcgttgtgtgcatgccatgtgggcccgagcgccctttgttcgtggctttgggttggaaaggtgaccgtgtgagctgtgcggtgttgtactacgtattagtataaatcatttttgggtactactccctccgtccaaagcttatttataatttgttgtactccaaccgtccgtcttatttaaaaaaaatataaaaaaaattaaaaaaataagtcacacataaaatattaatcatgttttatcatctaacaataaaaaatactaattataaaaaaatttcatataaaacggacagtcaaacattgtcacgaaaatctaatgtttgcctttttttttaagaccaagggagtatctacgaacaaagataatacatgttataatcatgaagcccatgatgtgattagcccggccgtttgactaacctcacgagctacgtggctgacaagtttaacttgttaactccatcatttcggatacttagagcatgtacaatagcagactattagccagctataaacatattttaatgggataaaagatgagagagaagagcagcgggctacagatttatagccagctgcagcacggactccaagacgcaatatgtgtatgacaggtaagaccatatgttaatagtatagtaagcaactattttataaactggctattagatcggctatagataaattggagctagtagtggactatactattcaacttgctcttatatgatataaatattgatataactatatgattttgttaatgacatgtttgtttatggatggactatgtggggtcggtcgcctccgtagctgaccaaaatacaaacttaaaacccctatctataaaaatctaacttttgtttataaatatagatataaaagttcataattagagcctcatcttttaaacgaaaagagtactatgaaaacaactcgtaatacaaagactaattacgacgaaaagaaaatagtactgacaagaggaaagcagtgaacttgcatactccctccgtaaaaaaaaccaacctagacacggatataacactatatatctagattcgttcgttgtaatgaagtgtcacctccgtatctaggttggttttttcgtacgaaagaagtatgagtaaatctaaagctatgtatacccttcgtcaaaaaaaaaaagtaaaccttgtactggtgcgtgtcacatcctaatataatattgttttttatggagggtgtacagttgaaaaaaattgatgtgttttaaggatgaaaaatattggtaatgttggctatgtaactctagaaaaaaaaatgcagtaataataaaatgctaatttgctggagtactagattatagacaatccagtccaggacacgacaccctccctactctctccacttccactctcaccggccaccgcgcgctctctctctctctctcccccttctcccgcaagattcttcccccaaatcccacccgatccaccgccgccgcccgctcgccggagtcccatcgctgccaccgccgccggagccgcggcccgacgcccgccgggcctgcttgctgtgtgtgtgaggaggtggagttgctcgcgctcgttcccgcggccacctccgcctgctgctgcttctgcttccgctggcattgcggggaggtcgtgtgccgggggacgtgggggctcgtgttggagcgcggctgccggtgaggtggggggtgcggcgcggcgcggctcgcgctcgtgcgccggtggcgcgggcgcggggggaagcgtacgggggagggggagtgtggcggcggcggcgcgcggggtagggacgggcgccgccaccaccaccggctcgttcgctggcaggcgctacgcgtccagatccgtacgccggtatgcttcgtctcgccgcaactctctccatttgattagtatcccctcgccgaaacgaggcctgtgaggcgcccgctttctggctggcttccctgtactcgctgcttgctcctgcctgttgggttaacccgtttccatcgaatttgggtaagcgaaacatcgcctcatatgggcatttggggttctggcagccttaggctcgccatccgtcgccgagcttccaagtgaccggcgcttgttggtatatttgcttgcttgttcctgtttggtggctgcgctaaatcttttgtgctgcattgaatttatgccacccatatacagcaaattactgagctgaaataattcggctaattaggtccagcaatatgacatctcgtggattgaatgctaagctgacattgtatcactgatgctggcttatatataggttgttgagaagtgaagatgtcgacaggtgaaaccctgcgtgcagagctatcatccaggacgccgcctttcggtttgaggctatggattgtgattggaatcagtatttgggtggtgatcttctttatactaggtttcatgtgcctctggtccatataccgaaggaagccgaagaagtcctttgataagattccagtatctcaaatcccggatgtttccaaggagattgcagtagatgaagttcgtgagcatgctgttgtcgaaaacttccgtgtgcaagaaagccacgcgatatcggtgcaggagaaacattacgagaaagattcagggaaaatgctggcacacttggttaggagtaaatcgagtgatgccgataatttgagccaatgcagctcggtgtaccaatgtgatagggctggtagctcgtattctggtgatgaaggcagctcgggcaatgctaggaggcacttttctcaatatgcaactgtctcagcatcccctctggttggtctcccagaattctctcatctgggctggggtcattggtttactctgagagatttggagcatgcaacaaatcggttttccaaggagaatgtcattggagagggtggatatggggtagtttaccgtggtcgactcataaatggaactgacgtcgcaataaagaagcttcttaataatatgtaagagatcctgaaatctattctgcgttttacagaacttgtgactccttctgatgccatcatattaattttcttttgatatggtgctgcaggggccaggcagaaaaggagttcagggttgaagttgaggctattggccacgtcaggcataagaatcttgtccgccttctaggatattgtgttgagggaatccacaggtaaagctatttatcaatcacctttgctgatggatggctagcttttgtttctactggcacattatttacttgcatagggatgtaggattgctcttggtctatgtccacctactcaccagattatctcaagggataggttattcctgactgcactccttatgctatcgattttttcccttccaaatctgatggtgggattcagcatgcccagtgacagattatgctcagtccacagaaaccttctttggaccaccattcttttaccatgaaaatgtggccatagctccgaaagctaggattcactagaagcgcacaactgcttattggtttgttagttggctataacaaggtcttactgaaatgtacttccatagttcattactttgtgaatgcctgttcttgttcttcacgtttcttctcatgcatgttcaattctaaatttgtattcatgatatgtccaagctactgtattctccaaagaaaatcagaagtccattcacctatgtattttccagttttccgccattttggatactgctctagaaacaagttaataatatagatatttatatggtttggccagtgctgcttaagtgaccatcgagatagaaattgcttaagaaatatactaagatgttgagtgtcaggtgttttcggataatcttgttaccaacaaataggtcctatgaatataatggtgtctgcttcacgtaattcaaaatccacactcagccaaaataatctgcaatagggtgttgaaaatatgattatgtttctcccttgttttcatcatgactacagaaatgaacaatgttgctacatcttgtaataatttgtggttttcaattgaacaaaacatccatcaaatgatatctacagcaatatattttgcacttctgagcacacaataggtttgagtgtattcgagtcatggtcattgatttaagctttttatttcactacataaccattgatttgagtgtatctaaggagttctgtttccacaagtactttatgttaatggtgtctccttatgctttggccatccaaactcattactgttgtttaatatttttagtggttagtggtgtccaaatctttctttgtgtacatcatactatgtttttgtagtctattaaacttccatcctatcatctgacttgttatattccaggatgcttgtatacgaatatgtgaataacgggaacttagaacagtggcttcatggtgccatgcgccaacatggtgttcttacctgggaagcccgaatgaaagttgttcttggaattgctaaagcgtaagaaacaaaccatcgtccccgtcaaaaagaaaagaattgttcttcactttagctcttttatatgtatatgtttagttgcataacccattttccataactgaattggtatacaggcttgcttatttacatgaagcaatagagccaaaagttgtacaccgggatatcaaatcaagcaacatactaatcgatgaagaattcaatggcaaactttctgattttggcttggctaagatgctgggtgcagggaagagccatatcacaactcgagttatgggaacttttgggtatgttgatatttttttggagttagtattaatctttcctatgcttagcttttactgttggaatgtgcagtacttcgcttattcatacagtataaaattttacatgctgcgaactttgtccttcgtatattataacaggtagctttctcattgctatcattgattcatttcaggtatgtggcccctgagtatgccaacacaggtctgttaaacgagaagagtgatgtctacagttttggtgtgctattactggaagcagtgactggtagagatccagttgattatggccggcctgctaatgaggtgagcatatatcctacaatctcatgcgtattatgtatgttacaaaagtccgtactattggaaattattttacggcaaaataacgtctatactaggagagacgaatttgcttcaggtgtatggctgtctggcagttgtctactgtctagttacccttgtctcacttttacagtctattgttttatttttcaggagctgactagctgtataccttgtcatatataacaacactgtaacgtggatgccttgcaggtgcatctagtggagtggctcaaaatgatggttggcacaagaagagctgaagaggtagttgaccctgacatggaggtcaaaccgaccattcgggctcttaagcgtgctctcctagtggcactgaggtgcgtcgacccagactctgagaaaagacctactatgggtcatgttgttcggatgctcgaggcagaagatgtcccatcccgtgaggtggtaacgctttctcctttcctgcaataacattcatcatattatatcattgcaataaatctgaagcttttgctgtaatcctactgaaggaccggaggagccggaggggcaacactgccaatgcagataccgagtccaagacaagctcaagcgaattcgagataagtggcgatagaagggactcagggccatcagcaaggtttcaactctaagaagacggtgatcatagtcaagaacaatggcttcaaaactctatgcagtaacatggtggttggcagagaaaaaggggtatttctggagggcattgcattttgtattgtaggtctgcatggcggtagagactggagagagcacagtgtctgatgatggatacccggagacctgtaattcccattcagtattctgtttgttagtcaagcagcttgtacagatcgttgtctgttccattttttcattcttctggtttttttgtttaggaggctcttggattaccagtacgaaccgctgtctcttttctagaatcaccaacatggaacctatcaatatttactactagtactacgacttgctttcttcttgctgagatctatcatgtactgtacataactgacgtgttcagctgcacttggacaagtagatgctcgttctgtatgtcgaatttacttgatgaggtcgagcattaagtaccatggctgcagccggcttctgtttagttgtgctgacatgcggcggcgacctcacgctgtgtggcccattcttgatcttgggccgaaactgtagcaacgggcgtacggcccatctatatcgggattgttcggcccgttgtagatgggccggatcgggattgcgacttacgtgcgacccatttcggttgggccggtggtccgctacttcatctagcagtggtcggcggcagggttcacaattccaatagaatccaaacattattggattgagttaaaaacacaaaccaatcggctttttgtcaggttcagaaaattttaaactgaattttaattttttgacaaaaatctatttagatttcgtctgtttttttaggtttgtcaacggattcagcgaaatccgatgatatcgctcgtgagtggatttttgatccggtatcgagattgtgaacccttgtcgcgcattgcctgacaaagacaaccagtgaagcgccgtgcgcgccgcgtgcgcgccgcgtgacgcgaagatgcgcaggaaggaacaagctggcaagcggcgcgcccatgacggcggcggcgacgacgacccgcgcgcgtgcgtgcgtcaacgcacgcgaccggccgagatccgtcagtggccgcggctatatataatacatcgtcgcctcacaccccccacacaccgagtcatcgctcgccggagttagagttcgtagcggcgaaggatatagccatatattatagatggcgattggtgttggtggctgctgcgccgtgctgctcgcggcggcgctgctcttctcctctccggccaccacatgtaagcacgcccatcttcttcttcttcttctttttttctttcttttttttttttttttggaaatgagccgcagctgacaaaaagatcactcacacatggatacactgtcgtgacactaaccaatgcctaagccattttgttttcttgttttggatttttctttttatgtgtatcacttttgcttgttgctcttgcagatgcttatgattccctggatccaaacggcaacatcacgataaaatgggatgtgatgcaatggactcctgatggctatgctgtaagtagcggtggcagtacaccaacatctctacctttattttcgtctcaacctgtacatttacactatcttgttctactacctctaataaaaaaatatatttgatgttttaaaatctattaagttctagagattaggaaagctacacatggttttatgttttgatactattaagtagtatattttataagttatattgaaggctggggtttcaaaagtttgactacactagatcttattcaaagcgtctaatgattactgaacggaggaagtatgaacttatagacttgaagttaaacagcatagccacatctcttcatgtatacttcatccgtttcatattataagattttctagcattatccatattcatatatgtgcgtctagattcattaatatctatatgaattgggcaatgctataaaatcttataacctgagaaacggagggagtatgtcgcaaacaacaacaacaataacaacgagcaaaatctgtatcgaatccggtttccctcttgtaactgtatcaaagatctgtcctctgaaacgtcccctgttcatcaggccgttgtcacactgtccaactaccagcaattccggcacatccagccaccggggtggcagctggggtggacatggcagcagaaggaggtgatctggtccatgtacggcgcgcaggccatcgagcagggcgactgctccatgtccaaggagggcagcaatgtcccccacagctgcaagaagcatcccaccgtcgtcgacctcctcccgggcaccccaatcgacctgcagatcgccaactgctgcaaggctggatcactgagcgcattcagccaggacccggcaaattctgccgcgtcgtttcagatc

SEQ ID NO: 28 - DNA, 53905 bp, Orza sativa Asominori
gatcagtgagtgagagtgatgtgctattgattttcgtctaggattttgctgtgctcttcttcttcttctcctctctaccaagaaagatcgatggaggagaatttgtaggacgcgtttctcacgaattacttagctgttaatgatcagcttgatgtgtacgatatgatggtgcagagtgaaagttgtgttgttcactggtggatcatgggatgggaatatgggattgttgtaagatgtaactcaagtgttttcttttttgggattacttttggtaataagagcttgggtgatcgaaaactacagatggtttttcttttaagttgtatgatctctgtagagtttttgagtaatttgtagttttgtaccctatcaaagatcatctctagctgcctctgagctctccaactctatatgtccatctctagtatatatgtcccatatttctgactgaaaattttcaagtcggttggttccctccgcctggatattctttcagctaattagattttttttaaatgataaatttgctaaaagcttgttcaaattcagctaagatctattcaaacttcaatttctctatcgaaattcccggaaatttcaattcaatcattccccaatacatgccgatttccgtaatattgaaccatgacatgtaaacaacgaaggaatcaagggcatatttagtttcatctcacatcgaatatacggacacacatttgaagtattaaatgcactctaataacaaaacaaattacagattccgccagaaaactacgagacgaatctattaagcctaattaatacatcattagcaaatgtttactatagcaccacattgtcaactcatgacgcaattaggcttaaaagattcgtctcgcagtttcctgacgaaccgtgtaattattattttttctacgtttaatactttatgtatgtgcccaaatattcaatgtgacaacgtgaaaatttttatttggaactaaataggccctaatattctttcaagatattagaatagttatccctctccacctccctgcacaaacagtgaacttctttctccttgggcacaggagtagtagcagctcccggaaacagaaagcaatcaagcaaagtcctgaacctgaagcatcctgaaaccagcagacggcagaaaccagtgggcgcaggcgatagcagtttttcgtggtccggcgtacagccaaaatactggccatcgggtgcctacatagaatgagtccactggacgcagctaccaccgtgtgtgctacactgaccgccgctgctcgtcgaccagttgtacggggctgacttattctgaatttctaatggtttatttgggggtttagaacactgaggggtgctttagatccaaagatgtgaagtttgggcgtgtcacatcgggtattatatatagtgtcgcacagggtgtttgggcactaataaaaatactaattattgatcctatacgataagctatataatactcgatgtgacacgccaaaactttacatccctgaatctaaacacccttttaaatagagtatttggtgtgaaatataattttgatttgggaagaaggtgagtgagatttggaaaaaaaaagcatttcaattaaaaaatttgccagcagtaaataaagaaactactcggttttgtaattaaagtgaggttttggcacttctttgccctaaactggcctccattttataaagtgagaaccgtgcagcaaaagcctgaaaaggcaaaaagaaagaaattgtagaggtttttcaggaggatacaactaggtgggtctctaactctctatgcagctgtggtctgtggagcaaaacgatgaaatggaagacgggacgttgacgagggtgaagaaaacgagcgtttgaccagcgtcaaccatggcgtgaacagtagcaccactaacctgaccgagaggttgaagaagatgcaatcaacggggtactatagttcccacgaatttcccagcaacaacgggttggttctcactactcacgaattccctgtggctcaacaactactagtacatccttttgtccattatgataaaagttctatcttaatttttatttacacgtttttcaaactgttttttaattttctatataaaaaatacttaaaatatcaaataaaatctatttttggagttttaaaaaactcaattaatcatatatattattgacttattttattttacgtggactaaaatatcttcatcttcatttaggttatgttcttttctcatcaagatacatgatacattagcatgtttttcaaactgttttttaattttgtatataaacttactctaaaatatcaaataaaatttacttttagggtttataaaagtaaaactcaattaatcattactaacttgtttcattttacgtggactaaaatatcttcatcttcatctaaggtggtgtttggatccaaggactaaattttaatccctatcacatcggatatttgacactaattagaagtattaaacatagattaatgatgaaacccattccataaccctggactaattcgcgagacgaatatattgagcataattaatccatgattagcctatgtgatgctgtagtaaacatgtactaattacggattaattaagcttaaaaaatttatcttacgaattagctctcatttatacaattaattttattgttagtttacgtttaatacttttaattagtatacatccgacgtaacactgatcgatacaaacaccaactaaatcgaaaatcaccgaatggctcgtcatcctcccacatgagatgccaagatggaacaccaacaatccaacggctaggaagcgccccatcccacccaccgcctaaccgccttcctatgcaagtgggtcccaccccttccttccttttttttttctttttacaaatccccttccctttcttggctagctagctagcttggcccaacgccacgagccgagccgagcacatccggagccaagccgagctcagcgcctcagctccccctcctcctcgtcccattcccggtttcctcctccgatttcccccaaatccgcacgcctctcccctccgcctccatttttcccgattcccaattcccaaatccggatcagccgcagccgcagcagcaaaaaatttcgaaatccaaatccaaacccatcccccccacgacgacgtcacccacatccccacccccgcgagacgagacgagacgactcccaaatctctctctcctctctcctatgcgcgccgccgccgccgccgcagcagcagcagctaggaggcggagcagcagcagcagcagcagctgagatgatcgtgcgcacctacggccgcagatcccgctccttctccgacgggggaggaggggagcgcggcggcggcggtgggttctcgtcgtcgcaagacgcgttcgaattcgacggggaggaggaggacgacctcgtcctgctggggtcgtcgtcgcagtcgtcgcacccgcccgcgccgtcgcaggagtcgtcgtcgatgtgggacttcgacgaggacccgccgccgccgccccggcggcggcgggggaggggtgggggtggggactacgcggagcccgccacggcggcggcggcggcggcggcggccacctcgctcatggaggcggaggagtacggcgagatgatggagagcgtggacgaggcgaacttcgcgctcgacgggctgcgcgccaccgcgccgaggcgggtgcgccgggccagcttcctcgcgctgctcgggatctgcgcctccgcgccgcgccgccgcgtcctccgggcccaggggtcggtacaccaaagaaccctccttttttttttcttacttgtctgcgctgtaagtaaagaataacaattcgcgttcttgctcttgcttcgcgggcaatcttggtgaggaatcttgttagggttatgaaattgggcagccagttcttgtttcttctgcgtaatcttggcggaaacagtgggattttgtacgattatggctccgtaatcggcatttctgtgggaaatgaaccacctttagggcatttgaccttcgaacagcatgcttggtgttgcaatccgtagctattgccttcatcttaggcacaagaacttgttctgaattatgatttaccaacttgtgtttgttttcttgttctgagttttcttgcttggttagggttagggttatcaccgtggtggtgcagaattagatgttcgctacttgtcttaacctctgccttgcccaatttggtaccgagtgttacagctgggtttaggaagtgtgatctttgagcatttctagcatgttggtctctttattttgctaatctcacatggttgtagaggaaggaagcatagtgactgatgatgaatgcctagatactagaaatacatctttattaactgaattaggattgcttgggtatctatgtagatatgactgtagaatgttactgctggaaatgctatccaatatccattgatctctagcctaatatatctctcgaggccaagagatcagtcaattttgaactttcaggagagtttctatttggtacttaatctcttttatttgttacttttggtgcctggctctcttttcatgattgctaagtagacaggtaaagttctacctaaaattattcttaaaagttcaaaatcgctttagattaaggagtgccagccagagccttaggcagagtcttataaaccaaaagcacaatgctacaatgttcacaaaacttttgtggaatttccacttgagctgtataaacatcgcaatctactgtgaataaaagaagcacttgatggaagttcatgttagcaaatgacatgttttctgtgaggaggttgattgcttgaactgttatggactcttgcaactttttattttacttcgtacccatttatgctaatgtgcacaaataaaattgctgagagtaaaaatgtacaacttgttacgcaccagcacacttcctatttgtatccattttcctgttgaatttcaaatgtattcaattgctgaaattgttccattcaacaaacacatattccgttaatgaaattattatacattgcgttttgttttcttactcacaagtgtcctcttttcttatatcctatagattggtgcaacaaattattgatgcaattttggttttgaacattgatgatcctccctgcactattggtgcagctgctcttctattcgttttggcaagtgatgtgagtacctctcaatcccatccttgtgcttctgtgcatgcttcattctattttttacgcatatcgattgttttcttttatataacagcccataaaaataatcacatcatggcaaagttatttatttctccagtacagttatataagtattcaccacttttccatgaatatcttggcatgtgattacaaagaagattatttaagaaagtccatgcttttatttcatcattttgtttgaagttgaactttaatttatggtgtaaatttcagttaatattgctagcagctcgtattctttaatggcataacttcacttgtgcttattctccaatatctcccttcttgttgttcaggttcaagaaaatcatttgttggattcagaatcttgtgtccattttcttcttaaattattaaatcctccagtgaatcttgttgattccaaagcaccatcgataggttccaaacttcttggaatcagtaaagttcaaatgcttaatggatcaaataaggattctgactgcatttcagaggaaatcctttcaaaagttgaagagattctcttaagctgtcaagagatcaagtcgctcgacaaagatgacaagaaaacaacaaggccagaactgtgtccaaagtggcttgctttgttgacaatggaaaaggcatgcttgtctgctgtttcagtggagggtaagttttaatcaaatttcttggtcatgatttccctttatgaccattataattatttttatgagccaaataagcagttgccataagttacatagcacctgtttacaatattcatgggtggtttgcttagccctttgcttcacctgcctttgattgatgacttccatccgtgttgcacaactgaattggagtaattgactgcactagaagcacctatggccattgtcatactaggaaggttttcccttatcaaatatttgattgttacagagacttctgacactgtgtccagagtcggaggaaattttaaagagacattaagggagttgggcggtcttgatagtatttttgacgttatgatggattgccattcaacattggaggtgagatctcgctaacatcgcatattttacacttcctttgttcaactctaaaggatggtgcaagttttgttcctttttgccattttagctttaatgtgcttgaagccacatgaaagcaatgcttgtccagatacatagccaaaggttgttatattttgggacatggaaaatgcttgaggtagtaactattttcatcaggacatggaaaattggctgcatcacaaattatgttgtttcatgttgcaaaatagttttttaatacttttttattctgcatgtggtgttagtgtcttacagtgattcctctgatgattatatcccccacgataataatacttgacatatctacaccaagtggacattattcatttggatgttacttttccagctatacttgctgttcttgcataaactttggagtaaattgcgtatccctttaagagataaactgcttggtgctcctatctgtgtactttttatgcccccaactaataatgcaatcatattacgctgataaactgaataaataaattaacaatatacttctggtggaaaccttgtgtatcagaatctcataaaggatacctcaacttcagctttggacctaaatgaaggaacatctttgcaaagtgccgctctcctcttgaaatgtttgaaaatattggaaaatgccacatttctaagcgatgataacaaggtaatgttccttatatattctgtttcagtttagtacccattttcttcttctgtaccatcttctcccctcatttgttctgtgcaaaatgtgcaaacagtgtgactttgtatttctgcttaacatttttctttttttcctgaaaagcagtataaactcttacactcattttgcttcttgcagacccatttgcttaatatgagtagaaaattgtacccgaaacgctcctcgctttcttttgttggtgtcattatcagtattattgagttattatcaggtatttttcttaataatacaatatgtccgctaacacaataaaatgttttaaacatccagtatgttaaagttgcagtctgacgcctattttgttttgctgcagctctttcaatactgcagaattcttctgttgtttccagctctacatatccgaaatcgtctaaagtctctcaacagagttgctctggtaataacaaacaccaaatttgtttgatcaactcgttggcttttctgtgcactgtttcaatatagtttggtcgccattcaagtctcactacagatgttgaacttgacctgacacggtggcaccaatatttataaaacgctacctgatatttttaatatttcatgtttcctgacccagattatcttgttggttcctcatataagtttaattagtgtcgttcttgaaactttgttatgcagcagatgtcatggggggaacttcatttaatgatggaaagcgcaagaactcgaagaaaaaaaaccttttgtcgaaccagacacgccatagttgcttatcttcaaaatcagaagtttctcatattactatatcttctggtagtgatgctggtctgtcacagaaggcattcaattgttctccatctatatcaagcaatggggcatcaagtggttcattaggcgagagacatagcaatggtggtgctttgaagttgaatataaaaaaggatcgtggcaatgcaaatccaattagaggctcaagtgggtggatttcaataagagcgcacagttctgatgggaactccagagaaatggcaaaaagacgccgtctatctgaaaatgtaatcaccgacagtggtggcggtgatgacccttttgcttttgatgatgttgatcaggagccttcaaattgggaactgcttggtccaaaaaagaaatcgcctcagaaacatcaagacaaatcaggaaatggagtgctagttgcaagtcatgaaccagaccaacctgaagatcttaatcagtcgggtacaacatctctttttagtgctaaagatgaatccagtcttttggaagactgcctcttggcatcagttaaggtaattaaatatgtttccttctgatctttcttgtttcttcttcaagagaatatacattcttgggtcacagtttctcggtttgtctttgtgactttgttgagtgacatattttgaattcacaaaatttccttttcaatatggctcctcaatctatagcatctgtcgtgtatgtattctgtacaaaatagtattgtaacatctcctagaagaaattggcaccatccatatcatacagtagcaatttatgagacgtgatcctgattggaggtttaggacagagcctcgagctaaattgctattgtattgtatctactatcttttagtacatgatatgtgctgggcactctgtgtctgagtgtagtgagtgcttaagtttacatagttcagctaacatgcatatgtaagacagtttatgattaaatttaagtgtagaaagaaggtactttcaaaagatttttaaggacaatataattgtttcaccgggactcatgcttgttctgactgtgagcctaatgttacctttacatgcccttacattgtctattttttatcgttttatgagatcttccaaacaacttgatctgtcttaatgtttttttgctagctcctttcttggatatctggtaaatggttaggccgaagtatgaactttgccttattgtttcaaagaaaatgtaacaactcctggaaaagtctaattttggttgccctttattttgctgaccgtattggcacacatctaattctgctgttcctttctggcaggttcttatgaacttagcaaatgacaacccatctggttgtgaattgattgcgtcatgtggtggacttaacaccatggcctccttgatcatgaagcatttcccctcattttgttttgtcgtggacaacaactataacacgagagatgtcaatcttgatcatgagttatcatcttctcaaaacagcaaggcacaccaggtcaaaattaagcaattgcgagatcatgaacttgattttctggttgccatattgggcttgcttgttaaccttgtagagaaggatagccttaataggtaagtccctcacatgcttccttccatttgctcaattcatatcagtgttactgttctggcagttccttggggtcaggactcagaaacatccaattaatgttcatgttctcttaacgactcagaaatactttataacctctccacagggtacggctttcatctgcccgtgttcctgttgatctatctcagaatccacagagtgaagagacacagagagatgtcatagcactcctctgttctgtattcttagcaagtcaaggtgctagtgaagcttctggaactatatcaccggtaattcaaaattcttcaagttccttttgtatgtagattatatctttgtaaaactcggcatttattacctgctctttgtttcaaaaagcagtattttattttgctccttagcataggtcagcagaacagttgatcttattcagaaaacaatattttgcatgtaacatactgttatctatgagatgaaaattaatgcatgtgtaataatgtcaatgataaatatttgctatctgaatccagtctaccaactctagttagaccgaaattactgaggttctatttcaaagaataatttagtgcaccatttgttcaactactatgaagtaaaatggtattcccttctattgacatcgggttagaagtgaaaggccatcttaatgcaatgttctcaatgccacaaacccacaaatttcattaacacatacagattattattaacatagctataaattggatttccagaagcttgagttgaatttattttgttacaattgaaagcactgggaacattagcatttttttttagttcttggttattgcaatttataatgttatacagaactgtgtacctcacaatgcattcattatgacattctatgaaccatttgattgactgttgcttgtaaacaacaggatgatgaggagtctttgatgcaaggagcacgggaagctgaaatgatgatcgtagaggcctatgcagcccttcttcttgcgtttctttcaactgaaaggtttgcaatctgtagttgatggattgttttattaatgtctaactacttgcataatgtcagcactatggcatttaacttatactgtctgttaactgcaacagcatgaaggttcgtggagccatttccagctgccttccaaataacagcttaaaaatccttgtgcctgcgctagagaaatttgtggtatgtctccataattcttgaactactgtttgtataaaaaagtatggatgatctttgaatttactccattttggaaatcattaatttttcatgtctgaggtgtgaggtgtcaccataattgtacttcccatccaggaagcctgtttgcaaaatttcacataaataaggaaaatttgaacttgtttcaagtttgaatagtaacaggatgttttatttctcaactggagaaaacattccggctgggacttttaacccttaaaatgctagtgtgctcccactgtaagattgtctgctgtcacatttgaaactttgtgtaatacctttatcactacccttgagatgagagacacaatctggtaccgagttaagttattgataactcccagttgaagtacagcaccaaatcaagccaacatgttggctacgtaattaaatgttctcttacaacagatagaggtaaaaagggagtttctaagtatctaacctcttaccctcttggcttagcactccaggcacaactctttcttaacttgcgatttaggacttgactctgagaatattgtgtgcccacactggttgagtgcatgcctatctaagctgctagtttttgttcattttgattaactctgaagctgcctgagcttattctgcttccatcatttattaatccatcatgtttctctttcagtcgttccatctgcagctcaatatgatcacagaggaaacgcactcagctgtcacagaagttatcgagaaatgcaaactttcatagaaagagtgaagaggggcctgtacagatcaactaacaacctctttgcagcaaaaaagcatacacacaagtgtttgtcttggcctggggctctgcagatggactgatactctgacctgcagtgggcttgggagctaacaatggtttcattctttttttttttatgttttcccctgttgtttttgctcatgttttgtgtaattttttcttctcatctagcgatgttatttttcttagcatgatgggagtagccctccttttttttttctctaattaagtgtaaagtagcaacagcatagggatgaatgttcagtgtagtgtgtggtgtttcagttattcagagacgtccatacagtttgtaccttgtgaccacacgtcttaatctgatgaagcttagaataaatcacatgttagcaatgcaatatcatctgcgtcttctctcactttggtggccatcaaattctgtgtagaagtgtatggttggtgtgctgttgcaaatgccgtattccgctctgttttgtggaagttaagaagtccctagttgaaataccgatttttcatgatctcggagattgatgcaactctgattgcagcatttctttttattagaatgtacactccatgctatcatgatgtttattgtttagtactacaagatttggttaaccattattttaatatcataataattttataaaatcttggagtaacaagttcataatacatgatagcataactttttgaggctagtctatgtatattgtctcctttgtttttaaactaagcactcaataaattattgatggctgtaattttctgaaggtttcaccggtttcggcccgtgctttataaatagcttcggcacaaaagacaaaacggtccctccaacacataaatggttgagtttacgttttcattatctttggtaaaatcaagtccaccacgtagacactcataacaaaagtttgaatatcctcagaaattttgacttgagtctatcttacctttgatatcggacatccaaccctccctccctccctgaactttatattattcatattacacctgaactttatattattcatattacaccctgaagtggttttcatttaattgcatacatgctgaaatagtttgacaacgtgagatgcacaaaatctacacgttcgtcttaagttgcaattcattttatcccttttctttttctctcttacataggaatatcaatagtactaattcacattacaatatagtataaattggtgatcgattattggcaatatactatattaaatattcaaaactagtcatttaagctgccaaataagtaaaccactatcgaaaaccacaatataaatggcattacaaaacttagggggttgaatatccaattttaaagttcatgatgctagaggaatttctatcaaaagtttatgggtacatatggactttttcctttttaaaagaagctattcttatcgtaaacgttaaatattttttgtactttattttttatgattgaaaaaaaaacttagttttcaaaatgattggtctgtatacaagcatcaattagacttaataaattcatctaacagtttcctggcagaaactgtaatttgtttttgttattagactacgtttattatttcaaatgtgtgtacgtatatccgatgtgacaaccaaacccaaaaattttccctaactccatgaggccttacagatatatttgatgggtgtaaagttttttaagttctttgggtgcaaagtttttaaagtatacggacacacatttgaagtattaaatatagacaaataacaaaacatattacatattctgcctgtaaacaacgagacaaatttattaagcctaattaatctgtcattagcaaacgtttactgcagcatcacattgtcaaatcatagcgtaattaggctcaaaaatattcgtctcgtaatttacatgcaaactgtgtaattggttttttttttcgtcaacatttaatactccatgcatgtccaaatatttgatgcgatctttttggccaaattttgttggaatctaaacaaggatcaaatttgctgaatttttccagacgtcacggcttgttcatccatcgttcgcatcgcgattcgccaccgacgccttggtttccaacgaattttatcatccgcttaaatacatccaaagctctccatcgccatcggcggccaacggcgaccgctccgctctacccaatccacccatccactcgccgccgccccctgatccaaagcctccgccgcgccgccgtcgagaggaggaggaggaggaggaggaggaggaggaggcgtgagcccctatggggaccctcctccggccgcgtccgctcgcccacgccgccggcgccggcgacgccacgccgtcgaccgcgcacggtagccacgcgcctctcgagaggccccccccccgccgctcgctgatctctcttctcatcctgtttgggtttgggtttgtgatttgggtgtttttttttttccgcagcggtggtggtgagcggtggccgcggccgtggcgtggagtgccagccgcatcgggtgcgccgccgcccgggtccgcaggttgcggtggcgacggcgagctggaggaggcggagggagaccgtggtgagatcggatttcgccgctggtggtgccgctaccatgggggattcgccgcaggcgctctcaggtttgcagcctcctccactctcttctcgcaaaatgtgttgctatgttcctctcgctgggctggcctcatagccattaatgtagtttgctggaacattacattcggaacgttgttggcaattgcttgacaaaatgtggaattgtggaggggagaaaaatcgtttgaacctgcagtgacaaaattgccatctataattttaaaactgaaggtgtggaaatcaaacataatcattgccagcacatcattcttgttaaccaccttgacatattgttggcttataacagttagctccacaccaacttggaaggtgtcaatggaatgtaagtataaattgaggataactggcagttgttaagactttctacagaacttgtagcagctaaaactagctattgtgcatttatgtttcatggaatttgagcggcaatggatatttcttactaagacgtataatgcaaaacaaaaaaaaaaaaaactatgtctatgcagtttacatgtaatgtgcggatgcaaataaaatcatgttcatggacaaactaatgggattcataccaaattccagaattgcatttcttatgtggttacttttgtttgttgatttggttaccagacatcgatgtggtttcaagggtcagaggggtttgcttctacgcggtgactgcagttgcagcaatctttttgtttgtcgccatggttgtggttcatccacttgtgctcctatttgaccgataccggaggagagctcagcactacattgcaaagatttgggcaactctgacaatttccatgttctacaagcttgacgtcgagggaatggagaacctgccaccgaatagtagccctgctgtctatgttgcgaaccatcagagtttcttggatatctatacccttctaactctaggaaggtgtttcaagtttataagcaagacaagtatatttatgttcccaattattggatgggcaatgtatctcttaggagtaattcctttgcggcgtatggacagcaggagccagctggtatggctgtagtctcatccctgctttcttaagtagacatatatacatttacagtatttggtaaataaacaagattttatgaatcatatatgattttggggaaaacacaaaactctctttgttggctgccttgaacatagttctgttcacacagttatagcaccttctttaaaatgaagaactttgttgcatacacataaggccaaaccacataatgaattttgtttatttctatctttgaatgttagcatcgtttttgtttaatgcatgatcgccttcctatatatttgtagtatgtcaacattgtattccatgctgagcataacaaatggtttgttaaaattcaggactgtcttaaacggtgtgtggatttggtgaaaaaaggagcatctgtatttttctttccagaggggactagaagcaaagatggaaagctaggtgcatttaaggttcagtaaccaaacttaggttacattacatctaatgagatttttatattcagtatataatgttaaccttctcatggtgtactgacgtggttataaatgtccccagagaggtgcattcagtgtggctacaaagaccggtgctcctgtgatacctattactcttctcgggacagggaaactgatgccttctggaatggaaggcatccttaattcaggttcagtaaagctcattattcaccatccaattgaagggaatgatgctgagaaattatgttctgaagcaaggaaggtgatagctgacactcttattctaaacggttatggagtgcactaaagaaagatggtgtttttttttattatatggaacctattcaaaggcacagacaggctttcaaggctaagcttgttacaggtactgatactagttactaattactttcgtaatcagtataaataagcttgtgtagtgtaatggcattgtacatttctgcacttggtaaatttacagaagaggcaagtaatattttagaggattgagtttattcacccagtcatatagttgaagaggcaagtaacctgtaagagaggactgaacattaacacctcttgttcgattaaaaatgaccaaagagcatcaaacatgtattcgaggctgttactttagatatggcccattaatttgtttagttgtctatgtacatcctagttggtgtaaatgccagttaccatttctatgatctaaaacaatcaactcttttagtatattttcaaaaacgaaaattcagtacacatgtatgaatcttaatattcttctctagctcgttacaaaagcaacaaaggcaccgtgtcagctggttcacattagctagtttgtacttagcattatccactagcaccttattttcatgcatatcatgctaatttgcttgcccacgttgagtgggaatttttttcatgttttataatttatatatgttttagacttctagtccacaatttatgtacttcatgttcctgagcctctagtatggctgatagcagactaggtgctgagtgctgtccttttttgcagactgaagagagaagaaatacaagactgtccattgttagtcagatttgtaaaaatagactctgatgtagtttacttttgcccctattttatttttaacaatacaaatatataacagatcctaagaacttatcttaatttaggagaagttgctcgtttcattaaattaaattgtgaagtaaaaatgtgtgctcgagtctgtcaatgcaatcctgtgttcttgtttgaagatatggtgtagggcaggccaggattgaacactgaatggtaagactgcttctgccttcagacgttattgctaaatttttagctacttgcagttagtgctgccacgccgattaagcagtagaacaaagtagttttgtcgtgcacaaatgagttatatttcattggaaatcgaagcgaaaacgaatcaaaagttagaagaaaaggggaaacttggtaattactccataaagagagtgcattttattggtaagatggtatccggaagctgtgagctccgggctgtatgtattctggcaaatttgatatgagatgctcgattattggcttaagttagcgatatcaaatttggggaagcaccaaaggaattattgtgaaggagttatgggtgcgtgacgttatctgctaggttcaaatccttgtggctatgaatatttatctgctaggttcaaatcctagtgactatgaatattaatgggtaaggtaagggatttattgttaattttagtttctttaagattgtgccatcggacgccattcggtaactgtaataatgctttgtattggattcacttgtgttacatgcacgcactaaacatgtgctttaccttttcatctgtttttgcgttctgggctagaaactcaaacgttgaattttccatggtctgctcaacttgacaattactgcgtgtcaagcgatcttatacgcatactatgcgcacaagtgattgtatacggatatgatgacagtataacgtgtgatattgatttttttaataaaaaaatgatgttcctttccttgatgaaggaacaaagactttttttaaaagaagggtattactaaaaacaaaaatgacaaaaacaaaatatcagtgcacatggcaagtgtgctcggcaattttttctctgtactttaaacaaaaatacttctatatgttcttttttataagggtggcacaaatcttttaaatgagccaaatatctacattggatttattaaaaactgtataaattataatttatactctgaaaggttgtgtgcatctctcttggagaaaatgtataagttgcaaacaaacattaatccacgttatgtaactttttttcgccggaaaggccgaaggaggcctgacggagcgtggggctcctcaccgggagaccgcgcaggcccccctttgccggttcggccggggactcagggtgaaattctaagctctctgtatgtggaaggttcgcgaccgtcgaaagagcataagacacgggcgatgtatacaggttcgggccgctgagaagcgtaataccctactcctgtgttttggggggatctgtgtatgaaggagctacaaagtatgagccagcctctcccttgttctgggttccgaatctggaaaagtccagtccagtccccccctctaagtgggcaaggtcctccttttatatcttaaggggataccacatgcaccatctccctcctttctgtggggacttaccctaccttttcataaatggacggagatttgtatagttgccgtccgaatgaccttctgataggacggcccatacctacctccacttccgccgaaagcaggtgcgacgtgggattatggctgtctgctgacgacatgaccagtgtcagactggtcacaaattgctcattcctgtccaccacgcgtcagtttagcaatctacatgttggcccttcttcacacaacatcttgcctgtaatggttaggatgaagcctggcatatatctaaccaggactaacgtgccatctctaggaggtaacacgctagctccagctggggacgagcgcctagaagccctcgtcctgacgggatggggcgaggcgtgcgtcagatcgcctgtcgccacctaacccgcgatctgaccggtctgtgactggtcacagaccggataaacgagtgcactgcacttcgttacatgccgcgtgacacgctcagccaaaccgcaataaatgtggttaggtgagccccgctgtgctcacctaacccatacacgcggagcaaaaacccacgaggggtcggggcgcctcggccctcggggccgaggcgggtgcggtccgaccccctcggggggactaagaggagggcgaacacatcaccctcgggcccgacgtcccccgagggtgccaggccacgtgggcgattgtgtctgcctcaaacctctagtcatgatactcctgatcccatgtcaccgacagtagcccccggcgttatgccagggcgatcgccctctttaagggaagcggtcgggcgtgacgccactcctaaggcctggtgacaggtgggaccggtctccacaattgggcagaaacccaacggtcacaaatcacgcacatcggcaatggtaactctactatcaataatgagcggtctcttcaagactgccacattactcgagtagcacacgaatctggacatggcgattcgtttcgtctggagatatggtaacgtcgctttggtcggcgagcgtaattaacgcgcgcacgatatgatctatctcgactgccacaaccgcatatccacctcatgcgccgcaagcgggcgaatgggattagtggaagcgtgggcgcgagaaacgagggggcgaaatagtgggcgcgagaagcgaggagccgggcacagcgttggcaagagtataaaggcactgaggaaaggatctgtttccttcctttcgccatcatttcccttgtcttcgccgcttgcgccctaactccttctttcctgtgctctactttcgccacacgcgctcgctctcaatcttctcttcctccggcgccatggcacggggctccgctctgctcgatggtagcgtgctgccgccttcccgcatcgtgagcgagaggcaggctgggctgccgcgccgcttcatgccggaatctgccaccggccgggagatagtcacgctgggtgagggacgcccggcgccagactacccggggcggtccgtcttctttctcccctttgcaatggcagggctggttccgccattttcttctttcttcatggatgttctgaagttctacgatctccagatggcgcacctcacccccaacgcggtgatgacattggccatcttcgcgcatctgtgcgagatgttcattggggtgcgcccatctcttcggctgttccggtggttcttcaccgtgcagtcggtgtcgccgccatcggtagttggtggctgctacttccagccatgggggccggtgctgaatcgctacatcccctgcgccctccgcaagaagtgggacgactggaagagcgactggttctacacccccctcgccgacgaagcgcgcctctgacttccgagccagcccccggcgcaggcctccagctggcgggcgccggtagatctgggggatggctatgacgccgtcctcgaccgcctggcgggcctacgatcccaggggctcacaggggccatggtgtacggcgactacctccgtcgtcggattgcgccgctccagcggcgcgctcggggcgcctgggagtacaccgggtccgaagactacatgaggacccaccagggagtcagatgggactgggctcctgaggatttcaagatagtggtccaacgggtgctgaatctcaactccatggaggcgtccctcattccccaaggaatcctccctctctgcagcgatccagaccgcgcctccatcctgaccattatgacggcggtcggggcctcagaggagtgagctccaaagggccacgacggcgcaggcgggagccgtaggggggatcaatctaccccgggagggggtcgtgcttctgggtctcgcgacggaggcccgaggagcagccgccctgccgacgcccgggggaagaggaagcagggaggaacacctcccccatctcctccccgagggggcggggcggtgcgtgccaacagcaggcgcccggagggggccgcgccgacatcgcagcccgagggggagcgcaagaagaagcggctccgcaagatgggggagacagaaccatctcggggaaaccttatttcccctccaaagtggtcgtttaaccgaccccctcgcaggttcgtctctcacccatcgtggctgtattcattctctcaacgcgagttttcactcacccatcttgttcgtcttctggtcttttcttctgtttcagcgagatcccgtcgcgtccctcccgccattccaagtccggccagtctgaggccgaggatccggcggccgcagaggcccggaggcgggaatctgaccggcgagaggccgcggatcgcctacgggaagccgaggaggccgcccaggaggccgcccgggctcgccagggcgaggaaaccgctcgggaggaggccgcccgggcccgccaggccgaggaagccgctcgggaggaggccgcccgagcccaccaggccgaggaagccgctcgggagaaagccggatttcgccaggacgaggcaatggcgacttccgaggcagctcgcgatgaggtcgcgggcgcgtcgcttgagcccgcttcctcgggcgacgctcaggcgacaacttccggggcagctggcgacgaggctgcgggcgcgtcgcttgggcccactccctcaggcgacgcccaggaccaaccaggtctgagggacatccccgagtccggcacttccatcggcggcccgagccgcgtggcatcctctccaaggcggctcttccccacgccttctatcgccccgctgagcgcagagccccttctgcaggccttggccgccgcaaacatcgcggtgttggacgggcttagtgcccaggtggaggccctgcaagcagagtgggcggagctcgacgccgcgtgggcgcgtgtcgaggaggggcggcgctcagtggaggccatggtggaggtgggccgcaaggcacaccgccggcatgtctcggagcttgaagcccgtaagaaggtgttggcggaaatcgccaaggaagtggaggaggagcggggggctgccctcattgccaccagcgtgatgaacgaggcgcaggacaccctccgccttcaatacgggagctgggaggcggagctagggaaaaagctcgacgccgcccagggggtgcttgacgttgccgctgcccgagaacagcgggcgggggagaccgaagcggcgtcccgacggcgcgaagagacccttgaggcgcgcgccatggcgctggaagagcgcgcctgcgtcgtggagagggatctggcggaccgcgaggccgccgtcactatccgggaggcaacactggcggcgcacgagtccgcctgtgccgaagaggagtccgcactccgcctccacgaggacgcgctcaccgagcgggagcgagctctcgaggaggccgaggccgcggcgcaacggctggcggacagcctgtccctccgcgaggcagcgcaggaggagcaggcgcgccgcactctggaatgtgtccgcgccgagaggaccgcactaaaccagcgggccgctgacctcgaggcgcgggagaaggagctggacgcgagggcgcgcagcggcggggcggctgcgggcgaaaacgacttagccgcccgcctcgctgctgccgaacataccatcgccgatctgcagggcacgctaaactcgtccgccggggaggtcgaggccctccgcttggcaggcgaggtagggcccggcatgctttgggacgccgtctcccgcctagatcgcgccggtcggcaggtgggcctctggagagggcggaccgtaaagtacgccgccaaccatggaggcctcgcccagcgcctctcgaagatggccggggctctccaacggctccccgaggagctcgagaagacaattaagtcatcctcgagggacctcgcccaaggagcggtggagctcgtactggcgagttaccaggccagggaccccaatttctctccatggatggcgctggatgagttccctcctgggaccgaggacagcgcgcgcgcaggtccgggatgccgccgaccatatcgtccacagcttcgagggctcagcccctcggctcgcgttcgcccccaactccgacgaggaggacaatgccggtggtgcagacgacagtgacgatgaggccggcgacccgggcgtatcggattgatcccccaagcccccgccattcttcagttttttcttcttttccttcttctaaggccttcgggcctcttttttgtatagatcaacttaatctgtaatcaaaaatgaagaaatttttgtgtcaatttcatcttgctgtgtgtatgagatgaggatgatctgtgacgtggtccttttgcgtcttagcttgattaagggctcgtgcccaggtcccagtcctcaaaaggcgtgggtcggggctagtgcctggggagatccacatgtcgagactggccaggccgggaacgtggtgaccgagggttatgggtgacccgattgtgggtttttgccgattcccccccggagttcaccacgccccggggcacggctcggttctgggccccgtttggcgattttagccgacccgagcccccgagggcaggattgagcacgagtgacctatttcaagtcaagattcttcaaaaggaaaaaaaaacacagatacagcctttaggaaattgaaactgcttttattgaaatactgaaataagagaaataagaatgtgcatgtgtggcagcccccggccaaccctgcacgcccgagggggtgcggggttggcccgagcccgaaacctgacacccgaccccccccctcaggggtagaagcgacgaaggtgttcgatgttccacgggttaggcagctcaatgccgtcgcccgtggccagccgtatggagcccggccgggggacgccgaccactcgatacggaccctcccacattggtgagagcttgctcaatccagcacgcgtttggacgcggcgtaggacgaggtcgtcgacgcagagtgatcgggcccggacgtgacgctgatggtagcgccgcaggctctgctggtagcgcgcggctctgagggccgcgcgtcgccttcgctcttccaagtagtcgaggtcatctctgcgaagctgatcttgatcagcctcgcagtacatggtggcccgaggagacctcagggtgagctcggatgggagaaccgcttccgcgccgtagacgaggaagaaaggcgtttccccggttgctcggcttggtgtagttcggtttgcccagagcaccgctggcaactcctcgatccatgaatcgccgtgcttcttgagtatgttgaaggtcttggttttaaggcctttgaggatttctgaattggcgcgctccacttggccattgcttctggggtgggcaggtgaggcgaagcagagcttgatgcccatgtcttcgcagtagtcgccgaagagttcactagtgaattgggtgccattatccgtaataatacggttaggcactccaaaccgggccgtgatgcccttaatgaatttaagtgcggagtgcttatcgatcttgacgaccggataagcctcgggccacttagtgaacttgtcgatcgcgacatacagatactcaaacccgcccggggcccgcctaaacggtcccaggatatcgagcccccagacagcaaatggccacgaaagtggtatggtctgcagggcctgggccggctgatggatttgcttggcgtggaattgacacgctctacatcgccggaccaggtcgaccgcatcattgagagctgtcggccaatagaaaccctggcgaaaagctttaccaaccaaggtgcgcgaggcggagtgggctccgcattcgccttcatggatatcggcaagaagcacaacgccttgttcccgaggaatgcacttcaggaggattccattagccgcgcgccgatagagggtcccttctaccagcacgtagcgtttggagatgcgatggacgcgttcactcccttcgcggtcctcgggtaaagtcttatctgtgaggtatgcttggatctcggcaatccaagcaatcaatctaagggagctgggagcgctcccctcgggtcccgaggcctggacttcaacgggcctcgggggccggtcaggcgcgtccgtctcccctaaggggtcgggtcgcgccgacggctgggcaagcctttcttcaaaggcgcccggtggggtctgggctcgcgtggacgcgagccgtgagagttcgtcggcaatcatgttatcccgtctgggcacatgccgaagctcaatcccgtcaaaatggcgctccatacgccgtacttggcgcacgtaggcgtccatctgcgggtcagagcaccggtactccttacagacttggttaacgaccagctgggagtcgcctaacaccaggaggcggcggatccccagtccagctgccactctgagtccggcaaggagtccctcgtactctgccatattgttggtcgctcgaaagtcgaggcggaccaagtatctgaggacgtctccgctcggagaggtcaacgtgacccccgcaccggcgccctgaagagacagggagccgtcgaactgcattacccagtgggcggtgtgaggcagctgcgaggggtccgtgctggcctcggggattgagacgggctcgggagccggggtccactctgccacaaaatcggcgagagcctggctcttgatagcgtggcgtggttcaaagtgcaaatcgaactcagaaagttcgattgcccatttcaccacccgtcctgtaccgtctcgattatgcaagatttgaccgagggggtaagacgtaaccacagtgacccgatgcgcctggaaataatggcgcagtttcctcgaggccatcagaatagcgtaaagcatcttctgggcctgagggtatcgggttttggcgtcccggagggcctcactaacaaagtagacgggccgctgcacctttcggtggggccgatcctcttcgctaggggccgcatccctggggcactcttcgtccaagcagcctcgcggggcgcacttgtcttctgtgctgatgacctcggggtcggaggataacaggggcggccttcccacagtggctttggggccgtcctgggggtcaggggctcctggcgtcgtcggacaagcgggcaaagggccaactccggtcgtcaggggccttaggcctccgttcggctcgggggcctcttctccctgctctttcccgggtcgagtcagcacagggttagcctcggggtcaaagggcgataggtgcggccttcccacagtggcctcagggccttcctgggggtcgggggctcctagcaccgtctgacaagcgggcagagggccaactccggtcgtcgggggcctcgggccaccgttcggctcgggggcctctcctccctgctctctcccgggccaagtcggcacagggtggggaagcgcgaaatgagaattgtcctcatcgcgctccacaaccaatgccgcactaactacttgcggggtcgccgctaagtagagtagcaagggctcgtctggctccggggcgaccagaactgggggagagcttagatacgccttcaactgggtgagggcattttcagcttccttcgtccaggtaaacggtccggagcgtttgagaagcttaaataagggtaacgccttctctcccagcctcgatatgaaccgacttagggcggccatgcaaccggtgacgtattgcacatccctaagtttgctgggggggcgcatccgctctatagcccgtatcttctcggggttggcctcaatgccccgggcagagaccaagaacccgagaagcttgcccgcaggtacaccgaacacacacttatcggggtttaattttatgcgggcggagcggagactctcaaaagtttccgctagatctatgagtaacgtttcctggttgcgcgtctttacaaccaagtcatcgacataagcttcaatattacgtcctaattggctacccaaagaaattcgagtagtacgttgaaaagtaggacctgcattctttaacccgaagggcattgtcgtataacaataggttcctatgggggtaatgaacgcagttttttcctcatcctccctagccatgcgaatctgatggtaaccagagtatgcatctagaaaacacaaaaggtcgcaccccgcagtggagtcgacaatctgatctatgcgaggcagggggtaaggatccttaggacatgccttgttaaggtcggtgtagtcgatgcacatccgaagcttgccgttcgccttgggaacgaccaccgggttcgctagccactcggcggggttgacgctgccatcatatttttcggcgatggtgggccggaaccttgggggccaacggacattccgaagactcgccacaaaggctctacagccgacaccaccaaccgggggcacggagggctgattcccgcgtccgtgttgaggtgacactctggacgaggaagcgccctccgttgcgtgggcagcacttcggtcattacgccggcgctcgatgctggtgcgggcgtccggccccccacgcagatctttctgggtcgaaggagtcgacgaaggagtggcggccgaatggcgaacagcggctgccgctcgtcgtgccctccgtcttgacgacgcggagccggtggtagcagcaccagaggccttggtggcggaggaccgcccaccagcatctaggcgctgccgtaccgtcatgactaatttggccacgtcgtccagccatcgttgggctggagactccgggtcagggacgacaggcgggtgacgtaagagcgcgcccgcagcttggagcgcgccctggggcgtgctgccgtcgccgtagacgaggaggcgacgctccccatctcgccgttcttctccatcgcccgcgatcggtgaagtcgcggatctttcgaccctctcgagcgcctccccccgcttaggactttggcgtggagggagcggtggagtacgagctcgacggcgtgggttcggctccccgtcgtcgccactcacactcggagagaggtcgtgcgcctttgcttgctcggccatcaggctgaacaggaaaagcttggcgcacacggaagagtacgagagctcagaaaaacacacactgagtcccctacctggcgcgccagatgacggagcgtggggctcctcaccgggagaccgcgcaggcccccctttgccggttcggccggggactcagggtgaaattctaagctctctgtatgtggaaggttcgcgaccgtcgaaagagcataagacacgggcgatgtatacaggttcgggccgctgagaagcgtaataccctactcctgtgttttggggggatctgtgtatgaaggagctacaaagtatgagccagcctctcccttgttctgggttccgaatctggaaaagtccagtccagtccccccctctaagtgggcaaggtcctccttttatatcttaaggggataccacatgcaccatctccctcctttctgtggggacttaccctaccttttcataaatggacggagatttgtatagttgccgtccgaatgaccttctgataggacggcccatacctacctccacttccgccgaaagcaggtgcgacgtgggattatggctgtctgctgacgacatgaccagtgtcagactggtcacaaattgctcattcctgtccaccacgcgtcagtttagcaatctacatgttggcccttcttcacacaacatcttgcctgtaatggttaggatgaagcctggcatatatctaaccaggactaacgtgccatctctaggaggtaacacgctagctccagctggggacgagcgcctagaagccctcgtcctgacgggatggggcgaggcgtgcgtcagatcgcctgtcgccacctaacccgcgatctgaccggtctgtgactggtcacagaccggataaacgagtgcactgcacttcgttacatgcggcgtgacacgctcagccaaaccgcaataaatgtggttaggtgagccccgctgtgctcacctaacccatacacgcggagcaaaaacccacgaggggtcggggcgcctcggccctcggggccgaggcgggtgcggtccgaccccctcggggggactaagaggagggcgaacacatcaccctcgggcccgacgtcccccgagggtgccaggccacgtgggcgattgtgtctgcctcaaacctctagtcatgatactcctgatcccatgtcatcgacaaggccatccgaatgtattaaggagtaaaagttacaagaaaaaacaccacaatgcaccaaggtgcatgaccacacaccatacactacccccaagcacaaaccactgagggtgaagcctagcaccaaacgaccgccactaagtgtgaccaaacgccgctaggcctacggcagcaacacatagatgagacttcgaaaacgatgccaccaaggtggtcacgacatgtaggatgctgccatcgtccatctaaaaagatgtggttttcacccagagaaactcatcaagaaggggagagggtaacccttgacagcgccccaaggaggttacgacgcccgaaggcgtagccgctgccggtccggtgaaccaccggactaggcttccgcctaggaccctatagccttgatcgcagatcaccgtccaccactcagaaccaccacacagacaaaaggtagcacgtagcttccaccgcaccgcaccgacgccccttcgtcggccgactccatcgaaccaccatccctgagagctggcccaggacccctccgttccaccacccgccggccgccttgccagttttggccaaaggagaacccgggactgggtgacattgcttcggcagcctgagcttcccccgctggcgagctgctgtctcaatccaacctagaaactccccgcaaaagaaggggatgagctctaggaagggcgagggtgccgaccggcaacgaggaagacaacccatcgactccagctccctttgcactaccatctgggcctgcgccaatgccggatacgctgtcgctccggctccggcgccacccacctgcaccccctttgcctggtctccgcgcccctcctggctgcgtcgcgccgcccagctggccgctaagggcaccacgacggccgcccggctaccgaggcctggccgcgccatgggacagctcgcgctggcaccagcgagccacggccgtcgcgctgttgccggcgccagcgagcacaaccgccagctccaagggccgagcatgccactgagccgccgccgctgccgcccgggccggctgcacgtcaccggcgcacacgaccgcacgccgccacgctccgcctccgcgcccgaggcagccccatgccattgccgcgcacctcgcccgcccgctgccgagccgccaccgcgcaccttgctgagccgccaccgccgtccctagccgcctcgtgccgccgccacgccagatccaggcgcgggatggccggatccggccttgggggcgccggatccgccgcctccccacaccgccacggcgtcaccacctccgaccgcagtgagggcttcgtcgtttgccccatcctcatcgcgtcgaggaggaagacgccaagaaaaaagggcctcgccgctgccttccttgctcgctgccggcttcgccgccggcgagctccggcggcggcgaggtgggggagaagaagtggggagtgggcagctagggttttttcgccccccaagccgcccgtgcgagagcgacggtggggggggggggactttccaacctcttccagtgttctagttctccacgttatgtaactcaatttgtttaaccatagaaagtaagaaacctaccagcgtgttaagctctctttcattccctttcttcttcctggttttgcttccatcacatgtcaagtgaagggttcttaactaccattactcctacacatctaatttttttctcagatctttcgcaggtatatattgatgctacattttatgatcttaagataatctccttcacattaccctctgctgaaactttagcttgaaccgtcatcttcaccacaatttgagcccaatttgcacagagcacaacgagcaatagcttgcccttacgttcattatttagcatgaactactactaactacccaagaatcaatacaccggtttaataacgccattttatcacgttaatatatgtttcattcaacacaccggttttggcacagttgcaaacttgcaataaattctttcctacttctccatcccataatataacaaattggtatgtctcgtctggtactaagttgctatattatgagatggagggagcacttcttttcttccaaaatataagaatatagtattggattagatattatctagattcacgaattcgattaggttgtctagatttatagttgtatgtaatgtataattcggtaataggttattacctctcgggatggagggagtagttttgacttttttttttcttataaatcgctttgatttttatattagtcaaattttatcgagtttaactaagtttatagaaaaaaattagcaacatttaagcaccacactagtttcattaaatttagcatggaatatattttgataatatatttgttctgtgttaaaaatgctgctatatttttctataaacgtagtcaaatttaaataagttagactaaaaaaaatcaaaacgacttataatatgaaatggaggaagtagtagactataacaaatttaaaccgtgctttgattttagagcatcactaatatgttagcaataatctatccctaaaatttattttttttcctaaactgaaaataggaagtggaaatactcctccatctaagagagagcctaaattcaataaaaaactaaaaaactaaaggtggatccctctattaaactaccgcaaaaaatttatgttttttttctcttccacgcgcgcagaacagatatctcgatcaagttagcatgtaaaatttttaaagagataccttatacgactccttccgtatttccaaaagcaaacggatttaaaatctgactcaaataaagatctatatatccaatttacatgacacatgtttcgccgaatttttatattaataataattaatatttttaaaattaaattattagcaatttgtttggaggatttatcaaaacaggatggacgttgtttataacagcgtctagacctagacgcgcttgcaaactgcggccacccttttatcacacaaatttttgacaatttgacactttccaaaaattaattttataaattaaccgtgaccaaaacttatttaaaaataatctttttgttgagcgcaaaatcgtatacttcagcgccaaatagcacggcgccgacctcccccttcccctcccctctatcctccactgctgccgcccacctctccgtatcagctgcgtcgcgttggtttccgccggcgctgctgctgctgcaccagtccgctagggcgggcgggcatggcgcgccgcgccgcttcccgcgtccgcgccggcgctgttggcgcccttcgctcggagggctcgacccaagggcgagggggccgcacggggggcagtggcgccgaggacgcacgccacgtgttcgacgaattgctccggcgtggcaggggcgcctcgatctacggcttgaactgcgccctcgccgacgtcgcgcgtcacagccccgcggccgccgtgtcccgctacaaccgcatggcccgagccggcgccgacgaggtaactcccaacttgtgcacctacggcattctcatcggttcctgctgctgcgcgggccgcttggacctcggtttcgcggccttgggcaatgtcattaagaagggatttagagtggacgccatcgccttcactcctctgctcaagggcctctgtgctgacaagaggacgagcgacgcaatggacatagtgctccgcagaatgacccagcttggctgcataccaaatgtcttctcctacaatattcttctcaaggggctgtgtgatgagaacagaagccaagaagctctcgagctgctccaaatgatgcctgatgatggaggtgactgcccacctgatgtggtgtcgtataccactgtcatcaatggcttcttcaaggagggggatctggacaaagcttacggtacataccatgaaatgctggaccgggggattttaccaaatgttgttacctacagctctattattgctgcgttatgcaaggctcaagctatggacaaagccatggaggtacttaccagcatggttaagaatggtgtcatgcctaattgcaggacgtataatagtatcgtgcatgggtattgctcttcagggcagccgaaagaggctattggatttctcaaaaagatgcacagtgatggtgtcgaaccagatgttgttacttataactcgctcatggattatctttgcaagaacggaagatgcacggaagctagaaagatgttcgattctatgaccaagaggggcctaaagcctgaaattactacctatggtaccctgcttcaggggtatgctaccaaaggagcccttgttgagatgcatggtctcttggatttgatggtacgaaacggtatccaccctaatcattatgttttcagcattctaatatgtgcatacgctaaacaagggaaagtagatcaggcaatgcttgtgttcagcaaaatgaggcagcaaggattgaatccggatacagtgacctatggaacagttataggcatactttgcaagtcaggcagagtagaagatgctatgcgttattttgagcagatgatcgatgaaagactaagccctggcaacattgtttataactccctaattcatagtctctgtatctttgacaaatgggacaaggctaaagagttaattcttgaaatgttggatcgaggcatctgtctggacactattttctttaattcaataattgacagtcattgcaaagaagggagggttatagaatctgaaaaactctttgacctgatggtacgtattggtgtgaagcccgatatcattacgtacagtactctcatcgatggatattgcttggcaggtaagatggatgaagcaacgaagttacttgccagcatggtctcagttggaatgaaacctgattgtgttacatataatactttgattaatggctactgtaaaattagcaggatggaagatgcgttagttctttttagggagatggagagcagtggtgttagtcctgatattattacgtataatataattctgcaaggtttatttcaaaccagaagaactgctgctgcaaaagaactctatgtcgggattaccgaaagtggaacgcagcttgaacttagcacatacaacataatccttcatgggctttgcaaaaacaatctcactgacgaggcacttcgaatgtttcagaacctatgtttgacggatttacagctggagactaggacttttaacattatgattggtgcattgcttaaagttggcagaaatgatgaagccaaggatttgtttgcagctctctcggctaacggtttagtgccagatgttaggacctacagtttaatggcagaaaatcttatagagcaggggttgctagaagaattggatgatctatttctttcaatggaggagaatggctgtactgccaactcccgcatgctaaattccattgttaggaaactgttacagaggggtgatataaccagggctggcacttacctgttcatgattgatgagaagcacttctccctcgaagcatccactgcttccttgtttttagatcttttgtctgggggaaaatatcaagaatatcataggtttctccctgaaaaatataagtcctttatagaatctttgagctgctgaagccttttgcagctttgaaattctgtgttggagttcttttctcctacagttgtattagaggagggatcttctctttatgtgtaaatagcgaggtatgtatgtcacctctccgaattatttttactctggttcctagacggtaaacaagcaattatgttctgcctttgatgccagaaaaaacacaaaagtttgtcgttatctctactaacggatcataaaggaatttgtaactggagtttcaaacttaatttgtctaggcagtagttttggcattagatccaacattgtgtaggattcatttgtgtgtatcaatctatagggtttcattaaatttcgttaatgtgtactgtttaggtgttgaatagtttgacttgttttttaactgaacaaaagatactgaaatcgttccattcaacaaacacatgttccgttaatgaaattattgtacgttaccttttgttttcttactcacaagtgtcctcttttcttatatcctatagattggtacaacaaattattgattcaattttggttttgaacattgatgatcctccctgcactattggtgcagctgctcttctattcattttgtgaagtgatgtgagtacctctcaatcccatccttatgcttctgtgcatgcttcattccaattttttacgcatatcgattgttttcttttatataacagtccataaagataatcacatcatgacaaagttatttatttctacagtatagttatataagtattcaccagttttccatgaatattttggcatgtgattacaaagaagattatttgagaaaatccatgcttttatttcatctttttgtttgaagttgaactttaatttatggtgtaaatttcagttattattgctagcagctcgtactctttaatggtataacttcacttgtgcttattctccaatatctcccttcttgttgttcaggttcaagaaaatcatttgttggattcagaatctggtgtccattttcttcttaaattattaaatcctccagtgaatcttgttgattccaaagcaccatcgataggttccaaacttcttggaatcagtaaagttcaaatgcttaatggatcaaataaggattctgactgcatttcagaggaaatcctttcaaaagttgaagagattctcttaagctgtcaagtgatcaagtcgctcgacaaagatgacaagaaaacaacaaggccagaactgtgtccaaagtggcttgctttgttgacaatggaaaatgcatgcttgtctgctgtttcagtagagggtaagttttaatcaaatttcttggtcatgatttccctttatgaccattatatttatttatatgagccaaataagcagttgtcaacttgtcataagttacatagcacctatttgcaatattcatgggtggtttgcttagcccttttcttcacctgcttttgattgatgacttccatctgtgttgcagaattgaattggagtagtggactgcactagaagcacctatggccattgtcatactaggaaggttttcccttatcaaatatttgattgttacagagacttctgacacagtgtccagagttggaggaaattttaaagagacattaagggagatgggaggtcttgatagtatttttgacgttatggtggattttcattcaacattggaggtgagatctcgctaacatcgcatattttacatttcctttgttcaactctaatggattgtgcaggcttgttccttttcgccattttagctttaatgtgcttgaagccacatgaaagtaatgcttgtccagatacatagccaaaggttgttatattttggggcatggaaaatgcttgaggtagtaactattttcatcaggacatggaaaattggctgcaacacaaattatgttgttttatgttgcaaaaatagttttttaatacttttttattctgcatgtggtgttagtatcttacagttcctctgatgattatatcccccacgataataacacttgaaacgataataacacttgacatatctacaccaagtgaacattattcatttggatgttacttttccagctatacttgctgttcttgcatgtgtaagcaagtttggagtaaattgcgcattaatttaaatgcttggtgttcctatctgtgtactttttattccccaactaataatgcaatcatattacgctgataaactgaataaataaattaacaatatacttctggtggcaaaccttgtgtatcagaatctcataaaggatacatccacttcagctttggaccgaaatgaaggaacatctttgcaaagtgctgctctcctcttgaaatgtttgaaaatattggaaaatgccatatttctaagcgatgataacaaggtaatgctccttatatgttctgtttcagtttagtacccatttccttcttctgtactatcttctctcctgatttgttctgtgcaaaatgtgcaaacagtgcgactttgtatgtctgcttaacaattttcttttcttcctgaaaaagcaatatgaactcttacattcattttgcttcttgcagacccatttgcttaatatgagtagaaaattgaacccgaaacgctccttgctttcttttgttggtgtcattatcaatactattgagttattatcaggtatttttcttaataatacaatgtgttcgctaacacaataaaatgttttaaacatccagtatgttaaagttgcagtctgacgcctattttgttttgctgcagctctttcaatacttcagaattcttctgttgtttccagctctacatatccgaaatcgtctaaagtctctcaacagagttactctggtaataacaaacaccaattttgtttgatcagttgatctcgttggcttttctatgcactgtctcaatatagtttggtcgccattcaagtctcactacagatgttgaacttggcctgacaccaaatatttataaaatgctacctgatatttttaatatttcatgtttcctgacccagattatcttgttggttcctcgtataagtttaattagtgacattcttgaagctttgttatgcagcagatgtcatggggggaacttcatttaatgatggaaagagcaagaactcgaaaaaaaaaaacttttgtcgaaccagacacgtcattgttgcttatcttcaaaatcagaagtttctcatattactatatcttctggtagtgatgctggtctgtcacagaaggcattcaattgttctccatttatatcaagcaatggggcatcaagtggttcattaggcgagaggcacagcaatggtagtggtttgaagttgaatataaaaaaggatcgtggcaatgcaaatccaattagaggctcaactggatggatttcaataagagcgcacagttctgatgggaactccagagaaatggcaaaaagactccgtctatcttaaaatgtaatcaccgacagtggtggtggtgatgacccttttgcatttgaccgccgcgtcggcgtcgccaccacgtaatcgcccacgtcgctgcccccgctgccacgtcgtcgaccgcgcacggtaatcacacgcatctcgaggccgccgctagctgatatcttctcatccggttgatttgtgattttggcgtttttgcagtggtgatggcggggggcgaccgtggccgaggcgtggagtgccatccgcatcagggtgtatcggccgcgctgctccgccctggtccgcaggctttggcggcgagctggcggcggagggagactgtggtgagatcggatttcgccgctggtggtgtcgctaccatgggggattcgccgcaggcgctctcaggtttgcagcctcctccactctcttcccttttttatttttttttctcgcaaaatgtgttgtgatgttcgtctcgctgggcaggcctcatagccattaatgtagtttgctggaacatttacatttggaacgttgttggcaattgcttgacaaaatgtggaattgtggaggggagaaaaatcatttgaacctgcagtgacaaaattgccatctctaattttaaaactgaaggtgtggaaatcaaacataatcattgccagcgcatcattcttgttaaccaccatgatatattgttggttataacagttagctccacaccaaccttgaaggtgtcaatagaatgtttagtataaattgaggagaacaggcagttgttaagactttctaaagaacttgtagcagctaatactagctattgtgcatttgtgtttcatggaatttgagcagcaatggatatttcttactaagatgtatgatgcaaaacaaaaaactatgtctatacagtttacatgtaatgtgcggatgcaaataaaatcatgtacatggacaaactcatgggattcataccgaattccagaattgcatttcttatgtggttacttttgttgttgatttggttaccagacatcgatgtgatttcaagggtcagaggggtttgcttctacgcggtggctgcagttgcagcaatctttttgtttgtcgccatggttgtggttcatccacttgtgctcctatttgaccgataccggaggagagttcaggaaaaaaatttgaaaatacccattttttgaaaaagatttacgtttatatacactagtatgaagaatttgcgaaaatataactaatccgcagatcggttatgcgggagcgcaacaaaagtatggcgtggcggcgcggagtggacggccgaggcgttcgcgcggaatggggctgcgggaccgagccagtctcgcttgccggtaacgcggaaccggtacgctcccgcagcgccagtgtgcggaaccgcggcgccaacatttttttactgcatggcactgtgtttaatactgtttgacactgtttctggtactgttttacacagttcccgggtcagttccgcacaatggaggcgcggcaccgaccatgaacaatgtgtgaacagtgctgcacagggttaaaacagtgtataaactgcgctgcacagtgctggagtcgctggccactgcggttccgcgttttggaaccgcgggaccgtcgcgattccgcgttttggagctgccggaccatgacggttccgcgcaggatcgtcggtcccgtattttgaatctgcggaaccgtcgctgtcccgcgtttccgtttcgcgggatgcgtatatttttataaaacctctccatgcatgtatataaacataaattattgaaaaaataagtatatttgcaaatttttttcgagagctcagcactacattgcaaagatttgggcaactctgacaatttccatgttctacaagcttgacgtcgagggaatggagaacctgccaccgaatagtagccctgctatctatgttgcgaaccatcagagttttttggatatctatacccttctaactctaggaaggtgtttcaagtttataagcaagacaagtatatttatgttccgaattatttgatgggcaatgtatctcttaggagtaattcctttgcggcgtatggacagcaggagccagctggtatggctgtagtctcatccctgctttcttaagtagacatatatgcaattacagaatttggtaaacaaacaagattttatgaatcatatatgattttggggaaaacaccaaactctctttggtggctgccttgaacatagttctattcacacagttatagcaccttctttaaaatgaagaactttgttgcatacacatatggccaaaccacataatgaattttgtttatttctatctttgaatgttagcaccttattttcatgcatatcatgctaatttgcttgcccacgttgagtgggaatttttttccatgttttataatttatatatgttctagacttctagtccacaatttatctacttcatgttcctgagcctctagtatggctggtagcagactaggtgctgagtgctgtccatttttgcagactgaagagaggagaaatacaggactgtccgttgttagtcagatttgtaaaaatagactctgatgtagtttattttagcccctattttatatttaacaatacaaatatataacgtatcctaagaacttatcgtaatttaggagaagttgctcgtttcattaaattaaactgtgaagtaaaaatgtgtgctcgagtctgtcaatgcaatcctgtgttcttgtttgaagatatggtgtagggcaggctaggatcgaacactgaatggtaagactgcttctgccttcatttgtgcacttggtgctgccacgccgattaagcagtagaacaaagtaattttgtcgtgcacaaatgagttatatttcattgaaaatcgaagtgaaaatgaaccaaaagatagaagaaaaggggaaacttggtaattatatactccacaaatttattggtaagatttgatattagacgctcgattacttggcttaagttaaggatatcaaatttggggaagcaccaaaggaattattgtgaaggagttgtgggtgcataacgttatctactaggttcaaatcctagtgactatgaatattaatgagtaaggtaagggatttattgttaattttagtttctttaagattgtgtccgggtacaccattcggtaagtgtaataatgttttgtattggattcacttgtgttacgtgcatgtgatttaccttttcatttgtttctgcgttctgggtatgaatttgacgagattccatggtcagctcaacatatcagttactgcgtgtcaagcgatcttatatggtatgcgcacaagcgattgtatacggatatgacagtataacgtgtgatattgatacgatgttcctttcctttataaaggaacaaagacttttttaaaaaaaagaaggggtattactaaaaaccaaaatgtcaaaaacaaaatatcagtgcacatggcaagtgtgcacgagcaatagcttgcccttacgttcattatttagcatgtactactactaactacgcaaaaatcaattcaccgattattaaactgttaacatcattttagcacgttaacatatgtttcattcaacacaccggttttggcacatttacaaacttgcaaagttgcaatactcccttcgttacatagcataagagattttaggtgaatgtgacacatctatccaaattcattatactagaatgtatcaccgcctccacgccgggagggagagcgccgccggtggagaaagggggagggagtggtcgaggggaaccagtagggtgccctccccgtcgccgcctccccgtggccgcgccggcgagacaggaggaagagggggatatggagcggcgccgccggtgagggcgcgcgcgcgggggggagcggcgacgccggtgaggaagggaaggggagtggtggctttgagagagataggggggaggaaaaatgattttagagttagggtttgggctgctgagtttttatatagatcgggatcaatcaggaccgtccatcagatcggacaactacggcttctcccgcgttgggccgggtgccactcctaggttgcccacactattgggccacatgtacgctccgcgtgaaataagttcactttaggtcctttaagttgcctctgaattgttcccaggccggccgcactattgggccaccccataggccatgtgtacgctccgcacagaataatttcgctttagctcccttaatttgtcccctcaaactcctaaaaccagtgcaaatctttaatttttagttcacccattgcaactcacgggcatatttgctagtgacatataatatgaaacgaaggatgtagcagactatagaatttaaactgtgctttcattttagagcatcactaactgttatttagatttttatttaaataaatgctgaaatgatgtttttattatgaaaattagcaataaagctcccaaaatttcaaaaaaaaattaaaagagatttattaatcatggttaatttaattaaaaattaaatctaaccatatcatattatttcacggtccgtgatgaggaaatggcagctgctatcacttacggtgggagagaaggggcattgtttatttttataactatctcttataactcccatgaaactataaaataaatataatcattatcataacattagttttttttccattgcaacgcaagggtaatttttcagtacaataaaaaaaataaaagtgggccattctgaacggaaatttctggttttttttcccaagagcgccgcacacaactgcgcaagagatcgatcgcgatcaccctgctcgtcgccgatctcctacaccatccctgccatctccttcccctccactggctgctgctgcacctgtcagctagggcgggcatggcgcgccgcgccgcttcccgcgctgctggcgcccttcgctcggagggctcgatccaagggcgagggggccgcgcggggggcagtggcggtggcgcggaggacgcacgccacgtgttcgacgaattgctccgtcgtggcataccagatgtcttctcctacaatattcttctcaacgggctgtgtgatgagaacagaagccaagaagctctcgagttactgcacataatggctgatgatggaggtgactgcccacctgatgtggtgtcgtacagcaccgtcatcaatggcttcttcaaggagggggatctggacaaaatgcttgaccagaggatttcgccaaatgttgtgacctacaactctattattgctgcgctatgcaaggctcaaactgtggacaaggccatggaggtacttaccaccatggttaagagtggtgtcatgcctgattgcatgacatataatagtattgtgcatgggttttgctcttcagggcagccgaaagaggctattgtatttctcaaaaagatgcgcagtgatggtgtcgaaccagatgttgttacttataactcgctcatggattatctttgcaagaacggaagatgcacggaagcaagaaagatttttgattctatgaccaagaggggcctaaagcctgatattactacctatggtaccctgcttcaggggtatgctaccaaaggagcccttgttgagatgcatggtctcttggatttgatggtacgaaacggtatccaccctaatcattatgttttcagcattctagtatgtgcatacgctaaacaagagaaagtagaagaggcaatgcttgtattcagcaaaatgaggcagcaaggattgaatccgaatgcagtgacctatggaacagttatagatgtactttgcaagtcaggtagagtagaagatgctatgctttattttgagcagatgatcgatgaaggactaagacctgacagcattgtttataactccctaattcatagtctctgtatctttgacaaatgggagaaggctgaagagttatttcttgaaatgttggatcgaggcatctgtcttagcactattttctttaattcaataattgacagtcattgcaaagaagggagggttatagaatctggaaaactctttgacttgatggtacgaattggtgtgaagcccgatatcattacccttggcaggtaagatggatgaagcaatgaagttactttctggcatggtctcagttgggttgaaacctaatactgttacttatagcactttgattaatggctactgcaaaattagtaggatggaagacgcgttagttctttttaaggagatggagagcagtggtgttagtcctgatattattacgtataacataattctgcaaggtttatttcaaaccagaagaactgctgctgcaaaagaactctatgtcaggattaccgaaagtggaatgcagattgaactttgttagatttaattggataattaatccatttaaatcaattaaatcaaataaattccaaggctcattatgctaggaattcatgtgaattcattcttctatgggatatcaatgggatgaagagttttgagaattaatccatttgattaaggaattggtaacttatatcaattaatcctaattgatggatggttgatggttgtgtagtggaggatggttcatggctagttgatgacaattagttgctctattcctcttcctattccattggtaacttacatcaattactcttaattgattgttggttgatggttgtgtagtggaggatggttcatggctagttgatgacaattagttgctccattcctcttcctattccatgactcttactcttcatcttccattcctcttataaaatgagaatggatttgatctcccgcgagaagaagaagacacactttcatccattttcaaaagctgttgctgctacggtaatcccatcccgacgagtgtgtgcacacgcgttgggagagtaggcctccgaaaccacgcgttgctgcgacgtttgcacagacgggcgggcgatcaggtttttggggagcgcaaggcgcgactactcactgttcgtcaacatctacttcatcttcaccaacatgtcgaacactggagacaaggagaaggagactcccgtcaacaccaacggaggcaatactgcctcaaactccagcggaggaccattcttggggtataaccttattacattatttcaattagaagttttactgttaatgttcatcgcaatgtcaacattgtgtcattatgtgattgttgatgcttattcaacgttaagcatgctcatgttgattacattcaccactatcactggatcaaatcctattgtaaatatcatgtttattatcttgttattttggattaaaatatgccgaattatgaccaaatttccaacaaacttagcacatacaacataatccttcatggactttgcaaaaacaaactcactgatgatgcacttcgaatgtttcagaacctatgtttgatggatttgaagcttgaggctaggactttcaacattatgattgatgcattgcttaaagttggcagaaatgatgaagccaaggatttgtttgttgctttctcgtctaacggtttagtgccgaattattggacgtacagattgatggctgaaaatattataggacaggggttgctagaagaattggatcaactctttctttcaatggaggacaatggctgtactgttgactctggcatgctaaatttcattgttagggaactgttgcagagaggtgagataaccagggctggcacttacctttccatgattgatgagaagcacttttccctcgaagcatccactgcttccttgtttatagatcttttgtctgggggaaaatatcaagaatatcatatatttctccctgaaaaatacaagtcctttatagaatctttgagctgctgaagcattttgcagctttgaaattctgtgttggaattcttttctcctacagtccgattagaggagggatcttctctgtatgtgtaaatagcgaggtatgtatgtcacctctccgaattattttgactgtggttcctggactgtaaacaagctattatcttctggtgttgatgccagaaaaaacacaaaagtttgtcgttatctctactaacggatcataaaggggtttgtaactggagtttcaaacttaaggtatctaggcagtaggtatatattgatcctacatcttatgatcttaagatgatatccttctcattatcctctgctgaaactttagcttgaaccgtcatctacaccacaatttgagccccttagcacagagcacaacgagcaatagcttgcccttacgttcattatttagcatgcactactactaactacccaataatcaatacatcggttattaaactgtttgtacagtttaataatgtcattttatcacgttaacatatgtttcattcaacaccacaccggttttggcacagttgcaaacttgcaataacatttttactacttctccgccccataatataacaatctcgttccatactatattgctatattacgggacggatgaagtacttctttccttccaaaatataagaatctagtcctagattagatattatttggattcacgaatttgattaggctatctagatttgtagtcgtatgtaatgtctaattcggtaataggttattacctctttggatggagggagtagtttttatttcgtactccctctgtttcatattataagttgttttgacttttttcttagtcaaattttattgagtttgactaaatttatagaaaaaaaattagcaacatttaagcaccacattagtttcattaaatgtagcatggaatatatttttataatatgtttgtttttttattaaaatgctactatatttttctataaatgtagccaaatttaaagaagtttgattacgaaaaaaaatcaaaatgacatataatatgaaactgaggatgtagcagactatagcaaatttaaactatgcttttattttagagcatcaccaaaagagatagcctaaatcttatcttaactaattaaaatattcataattttcctttcgtcacattaaattttcgtccgtaaatccgattgaaatccaactagacaatccaaaaaatagagaaaaagaacagaaaaaataataaaaagcacacaaatcttatctcaatcccgcgggaagctgccgatgccgccgaatccgctcgagcgccgccgccgccgctcacggggaacgatgtcgctgctatcgcacgtggtatgggagggcgccgccgccgctgcttgggagataggatatggagagagaaggaaatgtgagggagggttaggtttttccccattcgtatcttcagcgacacggaggcgatccaagctgtccatcagatcagacggctcagaacgcctccatcttcaggccgcgcatgcttgatgggccgagggaaggccggagggtcgaacaaacgtagtcagaggaggagttggaggaggtaaagtagaatttatttgcgggctgagatagtaaatggactgaaaatggcccatagagaaattgggaattttatttaaataaatgttgaaaaggtgtttatattatcaaaattagaaattaagctccgaaaattttaaaaaatattcaaagagcattattaatcatgattaatttaataaaaattaaatccaaccatatcatattatttcacggcgcgcagtaggaaaatgcgcagctgttgtcgcttacggtgggagagaagggacattgtttattttcagaactatcttttataactcccatggaactttaaaataaatataatcattattatagcattagtttttttctgtcttttttttccccaagagcgccgcgcagaagagatcgatcgcgatctccctgccccgacgtcgccggccgatctctcattctctccacgccctgctcgtcgccgatctcctacaccatccctgccatctcctccttcccctcccctctatcctccactggtgccgcccacctctccgtataagacaaactgcgttgcggcgttggtttccgccggcgctgctgctgcacctgtcagctagggcgggcatggcgcgccgcgccgcttcccgcgctgttggcgcccttcgctcggacggctcgatccaagggcgaggaggccgcgcggggggcagtggcgccgaggacgcacgccacgtgttcgacgaattgctccggcgtggcaggggcgcctcgatctacggcttgaaccgcgccctcgccgacgtcgcgcgtcacagccccgcggccgccgtgtcccgctacaaccgcatggcccgagctggcgccgacgaggtaactcccgacttgtgcacctacggcattctcatcggttgctgctgccgcgcgggccgcttggacctcggtttcgcggccttgggcaatgtcattaagaagggatttagagtggaagccatcaccttcactcctctgctcaagggcctctgtgccgacaagaggacgagcgacgcaatggacatagtgctccgcagaatgaccgagctcggttgcataccaaatgtcttctcctacaataatcttctcaacgggctgtgtgatgagaacagaagccaagaagctctcgagttgctgcacatgatggctgatgatcgaggaggaggtagcccacctgatgtggtgtcgtataccactgtcatcaatggcttcttcaaagagggggattcagacaaagcttacagtacataccatgaaatgctggaccgggggattttacctgatgttgtgacctacagctctattattgctgcgttatgcaagggtcaagctatggacaagccatggaggtacttaccacgatggttaagaatggtgtcatgcctgattgcatgacatataatagttatttcttgaaatgttggatcgaggcatttgtctggacactattttctttaattcaataattgacagtcattgcaaagaagggagggttatagaatctgaaaaactctttgacctgatggtacgtattggtgtgaagcctgatatcattacatacagtacactcatcgatggatattgcttggcaggtaagatggatgaagcaatgaagttactttctggcatggtctcagttgggttgaaacctaatactgttacttatagcactttgattaatggctactgcaaaattagtaggatggaagacgcgttagttctttttaaggagatggagagcagtggtgttagtcctgatattattacgtataacataattctgcaaggtttatttcaaaccagaagaactgctgctgcaaaagaactctatgtcaggattaccgaaagtggaacgcagattgaacttagcacatacaacataatccttcatggactttgcaaaaacaaactcactgatgatgcacttcagatgtttcagaacctatgtttgatggatttgaagcttgaggctaggactttcaacattatgattgatgcattgcttaaagttggcagaaatgatgaagccaaggatttgtttgttgctttctcgtctaacggtttagtgccgaattattggacgtacaggttgatggctgaaaatattataggacaggggttgctagaagaattggatcaactctttctttcaatggaggacaatggctgtactgttgactctggcatgctaaatttcattgttagggaactgttgcagagaggtgagataaccagggctggcacttacctttccatgattgatgagaagcacttttccctcgaagcatccactgcttccttgtttatagatcttttgtctgggggaaaatatcaagaatattataggtttctccctgaaaaatacaagtcctttatagaatctttgagctgctgaagcattttgcagctttgaaattctgtgttggaattcttttctcctacagtcctattagaggagggatcttctctgtatgtgtaaatagcgaggtatgtatgccacctctccgaattatttttactgtggttcctagactgtaaacaagcaattatgttatgctgttgatgccagaaaaaacataaaagtttgtcgttatctctactaacggatcataaagggatttgtgactggagtttcaaacttaatgtgtctaggcagtaattttgacattagatccaaaacaatttatagggtttcattaaatttcatctatgtgtactgtttaggtgttgaatagtttgacttgttttttaactgaacaaaagatatgtctgaagctttgttctttaccaaatgcagtactgatcatcacaatatattttttatggaacaagattggattgtatagaatggtttccgatctgattatcttatctcaacgtattattatgcacatgtactaatcatgaaatatctgatggaatgatgtttctatttacctgtgtgaggcagcaaggagtgagatggataacaccacatactccctctatcccagaatataagaagttttagagttggacacgattattaagaaagtaggtagaagtgagtagtggagggttgtgattgcatgagtagtggaggtaggtgggaaaagtgaatggtggagggttgtgattggttgggaagagaatgttggtagagaagttgttatattttggggagtacattattattctagaacaatactgttgtgctcaagaagcgttccaaagatgtttcacaacctgtgctcgatgggttttgagcttaatcctgggacattcagtatcatgatctgtctcattcttaaacatggaataaaggatgacagcatgatttctttgtctctataatcttttggctacccacagataatagctgtaaatctatactactttaaaaggagtagtggtggtggtgagtggtgaatctgccaccaccccaccaccaactctcaaaattctgacatgtgggatcactgtcaatcccttctccaagacatgtgggatcactgtcaatcccttctccaaaccaattgtatgatagaacagtggaaatcacggacagaccatggagctctcaaccataatcatccttgcgagttaataacaaatggagcgtaaacttggcaagcaaaaaactcaaattaattctaaaattaagctctaggattcaaaatagatttcctctctgcattgtgctgttatgatttttaattccgtaacaacgcaaatgcattttgctagtcttataaagaagggttaatgcaaatattctgattaaatgattgtatctatgaagtttgaatgctagtggaagctcctttgaccatgttttgttgtgcgagcatttaagagagtgaagagaatgcttctttggtgctgttctggtatggaaggatccacagataaaattcaggttctactgcttctctgcttgtaattttcatgaagctgcagtgaataccttgttgaccacttgatctgttgctttgaaggagaatatagtagtggccaaggttggtgacggtgatggtggcatgtgatcccccagatcttcagtgacccagagaggaggggacggcgcgtggtgagctacaaggcatactcagtggagggcaagatcaaggcctcccgtccgtaggggactccgctgcatcaaggccaactgctccgaactgatcaatttctggtacggatcacttctcctttccttttttttttcaccttaagcactctcttgattcttcgctgctacctcccttaatttctttcaatatattgtggcacttgatcatggcggagacccaccttccagtgtgaatggattttgtcaaagaactaaatttattccattagcttattttctgattacatggaagacattcttttctggaataaatacagaactaaatcctgtttcctgaataaaagttgttagtgtgtggcatggtgcatttccgcgcttctaaattttataaaacctgttcattcaatttgaacctgcatccaatccaatattttaggtgcagacaggtgcttgcggtcaggttaaagaagttggcaaaaatgcttctgaagaaaggttaattgttgtttcatctcaggaggtaatatgcagatgattattccaattggcattgccttgccatttttatcacgagtctttacaattttatatcctcctacatattctttccagattccagatgatccagtgtctccaacaattgaggcgcttattttgctccatagtaaagcaagtacacttgctgagaaccaccagttgacaacacggcttgttgtaccatcaaacaaagttggttgtattcttggggaaggtggaaaggtaattactgaaatgagaagacggactggggctgaaatccgagtctactcaaaagcagataaacctaagtacctgtcttttgatgaggagcttgtgcaggtaatttatttggccatacctacaccagagatccatatattacttttataactgcagtttttacttgttaacatttcattgtgcttttacatttgttccaagctttcaggttgctgggcttccagctattgaaagaggagccctgacagagattgcttcgaggctttgaactaggacactcagagatggaagttcttccaataatccgacaccttttgcccctgttgatggtcctcctgttgatatcttgcctaacaaggaattcatgctatatggacgatctgctaatagtcccccatatggagggcctgctaatgatccaccatatggaagacctgccattgatccaccatatggaagaccaatatccacaatatggaagacctgccaatgatccaccatatagaagacctgtcaatgatacatcatattgagggttggacaatgatgggcctcgtgatcaggcccggtcctgaggggggtcgaatggggcgatcgctccgggcccccgattcccagggcccccacctatctgtgcaacgagtagtagcgatcttccagcgcgcaacgtgaggcgatgtttctccgtgatttcgccggcctgcaactgcgagatcgcgagtataacgatcagccgatcgatctcatctgccgactgccatgctgatgccacacgcaagcgcagcatatcagccttatcttggttgatcggcatgctggacgagcacatctgttgtcgcatcaactgctgactgctatatatgtgctggtgctgaatcgatcgattgtcgtcgcggaagtgaagaacaaccacggcactgctgcctgctgggctctagccgccatcagtaagtacgctatactgcctatctagatctagatcgagattacatagtggaattatctgtttataacaaaattacaaggtatcaattgataatttaaggttataaccgtacaaacttcagtgatttgctggtttcacattggttagatttgtttcaactaatttggtacttctgtagccttgtaatttacgaatctagtattaatattttcttaagtattagcctgttccttgatattatgctgttgagaaagtatgcaatagataacaaaaacaagtaggtgtgttgaggatgctcaagagtaatacagccacttcaataattctgatattatcaggacatcatcaataattctgcgcctacaaatcttcaaagaaaattttaatataatgcgtatgattttttaaatacgaatattgattgctatttaaagatatttatattatatggtaattattatttgaaggtttataataaaggcctccgtttttagtttcacgctgggccttcagaatctcaggaccggccctgctcatgatc

SEQ ID NO: 29 - DNA, 24 bp, Artificial Sequence
  Oligonucleotide primer for amplification
atcaggagccttcaaattgggaac

SEQ ID NO: 30 - DNA, 24 bp, Artificial Sequence
  Oligonucleotide primer for amplification
ctcgcaaattgcttaattttgacc

SEQ ID NO: 31 - DNA, 24 bp, Artificial Sequence
  Oligonucleotide primer for amplification
tgaaggagttatgggtgcgtgacg

SEQ ID NO: 32 - DNA, 24 bp, Artificial Sequence
  Oligonucleotide primer for amplification
ttgccgagcacacttgccatgtgc

SEQ ID NO: 33 - DNA, 24 bp, Artificial Sequence
  Oligonucleotide primer for amplification
gcgacgcaatggacatagtgctcc

SEQ ID NO: 34 - DNA, 24 bp, Artificial Sequence
  Oligonucleotide primer for amplification
ttacctgccaagcaatatccatcg

SEQ ID NO: 35 - DNA, 24 bp, Artificial Sequence
  Oligonucleotide primer for amplification
aaggcatactcagtggagggcaag

SEQ ID NO: 36 - DNA, 24 bp, Artificial Sequence
  Oligonucleotide primer for amplification
ttaacctgaccgcaagcacctgtc

SEQ ID NO: 37 - DNA, 24 bp, Artificial Sequence
  Oligonucleotide primer for amplification
tggatggactatgtggggtcagtc

SEQ ID NO: 38 - DNA, 24 bp, Artificial Sequence
  Oligonucleotide primer for amplification
agtggaagtggagagagtagggag

SEQ ID NO: 39 - DNA, 24 bp, Artificial Sequence
  Oligonucleotide primer for amplification
ccctccaacacataaatggttgag

SEQ ID NO: 40 - DNA, 24 bp, Artificial Sequence
  Oligonucleotide primer for amplification
tttctgccaggaaactgttagatg

SEQ ID NO: 41 - DNA, 24 bp, Artificial Sequence
  Oligonucleotide primer for amplification
gcgatcttatacgcatactatgcg

SEQ ID NO: 42 - DNA, 24 bp, Artificial Sequence
  Oligonucleotide primer for amplification
aaagtctttgttccttcaccaagg

SEQ ID NO: 43 - DNA, 26 bp, Artificial Sequence
  Oligonucleotide primer for amplification
gaggatttatcaaaacaggatggacg

SEQ ID NO: 44 - DNA, 24 bp, Artificial Sequence
  Oligonucleotide primer for amplification
tgggcggcagcagtggaggataga

SEQ ID NO: 45 - DNA, 24 bp, Artificial Sequence
  Oligonucleotide primer for amplification
aagaagggagggttatagaatctg

SEQ ID NO: 46 - DNA, 24 bp, Artificial Sequence
  Oligonucleotide primer for amplification
atatcaggactaacaccactgctc

SEQ ID NO: 47 - DNA, 24 bp, Artificial Sequence
  Oligonucleotide primer for amplification
acgagtagtagcgatcttccagcg

SEQ ID NO: 48 - DNA, 24 bp, Artificial Sequence
  Oligonucleotide primer for amplification
cagcgtgaaactaaaaacggaggc

SEQ ID NO: 49 - DNA, 24 bp, Artificial Sequence
  Oligonucleotide primer for amplification
atcccacatcatcataatccgacc

SEQ ID NO: 50 - DNA, 25 bp, Artificial Sequence
  Oligonucleotide primer for amplification
agcttctcccttggatacggtggcg

SEQ ID NO: 51 - DNA, 24 bp, Artificial Sequence
  Oligonucleotide primer for amplification
atttgttggttagttgcggctgag

SEQ ID NO: 52 - DNA, 24 bp, Artificial Sequence
  Oligonucleotide primer for amplification
gcccaaactcaaaaggagagaacc

SEQ ID NO: 53 - DNA, 24 bp, Artificial Sequence
  Oligonucleotide primer for amplification
cctcaagtctcccctaaagccact

SEQ ID NO: 54 - DNA, 24 bp, Artificial Sequence
  Oligonucleotide primer for amplification
gctctactgctgataaaccgtgag

SEQ ID NO: 55 - DNA, 24 bp, Artificial Sequence
  Oligonucleotide primer for amplification
tggatggactatgtggggtcagtc

SEQ ID NO: 56 - DNA, 24 bp, Artificial Sequence
  Oligonucleotide primer for amplification
agtggaagtggagagagtagggag

SEQ ID NO: 57 - DNA, 24 bp, Artificial Sequence
  Oligonucleotide primer for amplification
tacgacgccatttcactccattgc

SEQ ID NO: 58 - DNA, 24 bp, Artificial Sequence
  Oligonucleotide primer for amplification
catttctctatgggcgttgctctg

SEQ ID NO: 59 - DNA, 26 bp, Artificial Sequence
  Oligonucleotide primer for amplification
acctgtaggtatggcaccttcaacac

SEQ ID NO: 60 - DNA, 26 bp, Artificial Sequence
  Oligonucleotide primer for amplification
ccaaggaacgaagttcaaatgtatgg

SEQ ID NO: 61 - DNA, 24 bp, Artificial Sequence
  Oligonucleotide primer for amplification
tgatgtgtttgggcatccctttcg

SEQ ID NO: 62 - DNA, 24 bp, Artificial Sequence
  Oligonucleotide primer for amplification
gagataggggacgacagacacgac

SEQ ID NO: 63 - DNA, 26 bp, Artificial Sequence
  Oligonucleotide primer for amplification
tcctatggctgtttagaaactgcaca

SEQ ID NO: 64 - DNA, 24 bp, Artificial Sequence
  Oligonucleotide primer for amplification
caagttcaaacataactggcgttg

SEQ ID NO: 65 - DNA, 24 bp, Artificial Sequence
  Oligonucleotide primer for amplification
cactgtcctgtaagtgtgctgtgc

SEQ ID NO: 66 - DNA, 24 bp, Artificial Sequence
  Oligonucleotide primer for amplification
caagcgtgtgataaaatgtgacgc

SEQ ID NO: 67 - DNA, 24 bp, Artificial Sequence
  Oligonucleotide primer for amplification
tgcctactgccattactatgtgac

SEQ ID NO: 68 - DNA, 24 bp, Artificial Sequence
  Oligonucleotide primer for amplification
acatactaccgtaaatggtctctg

(19) United States
(12) Patent Application Publication
(10) Pub. No.: US 2006/0253931 A1
(43) Pub. Date: Nov. 9, 2006

(54) Method of imparting or controlling fertility with the use of fertility restoring gene for rice bt-male sterility cytoplasm and method of judging the existence of fertility restoring gene

(76) Inventors: Toshiyuki Komori, Shizuoka (JP); Shozo Ohta, Kanagawa (JP); Nobuhiko Murai, Tokyo (JP); Yukoh Hiei, Shizuoka (JP)

Correspondence Address: BIRCH STEWART KOLASCH & BIRCH, PO BOX 747, FALLS CHURCH, VA, 22040-0747, US

(21) Appl. No.: 10/489,915

(22) PCT Filed: Sep. 13, 2002
(86) PCT No.: PCT/JP02/09429

§ 371 Date: Mar. 18, 2004

(30) Foreign Application Priority Data
Sep. 19, 2001 (JP) 2001-285247
Oct. 4, 2001 (JP) 2001-309135
Jun. 26, 2002 (JP) 2002-185709

(51) Int. Cl. A01H 5/00; C12Q 1/68; C07H 21/04; C12N 15/82; A01H 1/00

(52) U.S. Cl. 800/287; 800/320.2; 435/468; 435/6; 536/23.6

(57) ABSTRACT

The purpose of the present invention is to provide a method for providing and inhibiting the rice fertility to the rice BT type cytoplasmic male sterility, and discerning the presence of the rice restorer gene. The present invention employs a nucleic acid having the base sequence of SEQ ID NO.27, or a nucleic acid having a base sequence which is identical to at least 70% of the base sequence of SEQ ID NO.27, and which functions to restore fertility. Alternatively, a nucleic acid having the base sequence of bases 38538-54123 of SEQ ID NO.27, or having a base sequence which is identical to at least 70% of the base sequence of bases 38538-54123 of SEQ ID NO.27, and which functions to restore fertility, is used.

### FIELD OF THE INVENTION

The present invention relates to a method for providing and inhibiting the rice fertility, and discerning the presence of the rice restorer gene by using the rice restorer gene to the rice BT type cytoplasmic male sterility.

The present application claims priority based on Japanese Patent Application No. 2001-285247 filed on Sep. 19, 2001, Japanese Patent Application No. 2001-309135 filed on Oct. 4, 2001 and Japanese Patent Application No. 2002-185709 filed on Jun. 26, 2002. The entire disclosures of the three patent applications are incorporated herein.

### PRIOR ART

Rice is a self-fertilizing plant, so in order to perform crossing between varieties, self-fertilization must first be avoided by removing all stamens in a glumaceous flower just before flowering and, then fertilization is effected with pollens from the parent variety with which it is to be crossed. However, this manual crossing method is entirely unsuitable for producing a large quantity of hybrid seeds on a commercial scale.

Accordingly, hybrid rice is produced by the triple-crossing system which makes use of cytoplasmic male sterility. In the triple-crossing system, the following three lines are employed, i.e., a sterile line having male sterile cytoplasm, a restorer line having Rf-1 gene and a maintainer line having the same nuclear gene as that of the sterile line but not having any sterile cytoplasm. By using these three lines, (i) hybrid seeds can be obtained through fertilization of the sterile line with the pollen of the restorer line whereas (ii) the sterile line can be maintained through its fertilization with the pollen of the maintainer line.

When employing the BT type male sterile cytoplasm in the triple-crossing system, it is important to breed rice of the restorer line and to this end, it is necessary to ensure that the rice at every stage of breeding maintains Rf-1 gene and that the Rf-1 gene is homozygous at the final stage. It also becomes necessary in the triple-crossing system to check to ensure that the variety used as the restorer line possesses Rf-1 gene, or to check for the presence of Rf-1 gene in order to ensure that the resulting hybrid seeds have restored fertility.

In order to genotype the locus of Rf-1 gene in a plant, it has been necessary that F1 plants be first formed from hybrid seeds obtained by crossing the plant to be genotyped to a standard line and then self-fertilized, followed by investigating the incidence of individuals that can produce seeds at a frequency higher than a certain level (e.g. 70-80% or more). The standard line refers to the maintainer line, the sterile line or a set of the two lines, and it is appropriately chosen depending upon whether the cytoplasm of the individual under test is of BT type or normal type or unknown. If the standard line is a sterile line, it is crossed to the individual under test as the female parent and if the standard line is a maintainer line, it is crossed as the male parent.

However, these techniques require a huge amount of labor and time to carry out. As a further problem, fertilization for seed production is sensitive to environmental factors and if an investigation is made in an unfavorable environment such as cold climate or insufficient daylight, sterility may be caused irrespective of the genotype constitution, with the result that genotyping of the locus of Rf-1 gene cannot be performed accurately.

With a view to solving these problems, it has recently been proposed that Rf-1 gene be checked for its presence by a technique of molecular biology. The technical idea of this technique lies in checking for the presence or absence of Rf-1 gene by detecting base sequences linked to Rf-1 gene (such sequences are hereunder referred to as DNA markers). Note that it is not possible to directly detect Rf-1 gene since the DNA sequence of Rf-1 gene has not been clarified so far.

For example, it has been reported that the locus of Rf-1 gene in rice is present on chromosome 10 and located between DNA marker (RFLP marker) loci G291 and G127 which can be used in restriction fragment length polymorphism analysis (RFLP) (Fukuta et al., 1992, Jpn J. Breed. 42 (supl. 1) 164-165). This is a known method of genotyping the locus of Rf-1 gene by investigating the genotypes of DNA marker loci G291 and G127 which are linked to Rf-1 gene.

However, the conventional molecular biology techniques have several problems. First, they use RFLP markers which need to be detected by Southern blot analysis. In order to perform Southern blot analysis, DNA at the microgram level needs to be purified from the individual under test and, in addition, there is a need to carry out a sequence of steps comprising treatment with restriction enzymes, electrophoresis, blotting, hybridization with a probe and signal detection; this not only involves considerable labor but it also takes about one week to obtain the test results.

The second problem is that since the gene map distance between RFLP marker loci G291 and G127 is as long as about 30 cM (corresponding to about 9000 kbp in rice DNA), the probability for the occurrence of double recombination in the region would be a few percent and hence, it is not always guaranteed that the genotype of the locus of Rf-1 gene can be estimated correctly by the markers.

Thirdly, when the presence of Rf-1 gene is estimated by detecting RFLP marker loci G291 and G127, not only Rf-1 gene but also the gene region between those loci are introduced into the fertility restorer line selected as the result of breeding. As a consequence, the introduced DNA sequence will have a chromosomal region of 30 cM or longer from the Rf-1 gene donor parent, and this presents the risk of introducing a deleterious gene that may potentially be present within that region.

In order to solve these problems, there have been developed a dominant DNA marker (Japanese Patent Public Disclosure No. 222588/1995) and a co-dominant DNA marker (Japanese Patent Public Disclosure No. 313187/1997), both of which are linked to the locus of Rf-1 gene. These markers are linked to the locus of Rf-1 gene, their genetic distances from Rf-1 gene respectively being 1.6±0.7 cM (corresponding to about 480 kbp in rice DNA) and 3.7±1.1 cM (corresponding to about 1110 kbp in rice DNA), and their loci being on opposite sides of the locus of Rf-1 gene. Hence, the presence of Rf-1 gene can be estimated by detecting the presence of both the locus of the dominant PCR marker and that of the co-dominant PCR marker. The use of the co-dominant PCR marker also enables us to estimate as to whether the locus of Rf-1 gene is homozygous or heterozygous.

However, the use of these PCR markers still involve several problems. The co-dominant marker has a genetic distance of 3.7±1.1 cM from the locus of Rf-1 gene, and the problem of potentially high frequency of recombination with the locus of Rf-1 gene has not been fully dissolved. As a result, speaking of the co-dominant marker itself, correct detection can be made as to whether it is homozygous or a heterozygous. However, if recombination occurs between the locus of the co-dominant marker and that of Rf-1 gene, the genotype of Rf-1 gene locus cannot be determined correctly, particularly as to whether it is homozygous or heterozygous. On the other hand, if the dominant marker is used to genotype the locus of Rf-1 gene, the marker will detect individuals indiscriminately irrespective of whether they are homozygous (Rf-1/Rf-1) or heterozygous (Rf-1/rf-1) with respect to Rf-1 gene. Therefore, even if the co-dominant marker is used in combination with the dominant marker in order to genotype the locus of Rf-1 gene, it is not possible to correctly distinguish individuals having Rf-1 gene homozygously from those having the gene heterozygously. Further, if no amplification product is obtained in PCR using the dominant marker, one cannot deny the possibility that this is due to some problems in the experimental procedure. As a further problem, since the genetic distance between the co-dominant marker and the dominant marker is as great as about 5.3 cM (around 1590 kbp), the size of the chromosomal region introduced from the Rf-1 gene donor parent cannot be limited to a sufficiently small value to prevent any concomitant introduction of a deleterious gene which may be contained in that region.

Japanese Patent Public Disclosure No. 139465/2000 describes co-dominant PCR markers that were developed on the basis of the base sequences of RFLP markers located in the neighborhood of Rf-1 gene on chromosome 10 of rice. However, most of those PCR markers are spaced from the Rf-1 gene by a genetic distance greater than about 1 cM.

### SUMMARY OF THE INVENTION

An object of the present invention is to provide methods for restoring rice fertility. A method of the present invention comprises introducing a nucleic acid into rice, wherein the nucleic acid has the base sequence of SEQ ID NO.27, or has a base sequence which is identical to at least 70% of the base sequence of SEQ ID NO.27, and which functions to restore fertility. Another method of the present invention comprises introducing a nucleic acid into rice, wherein the nucleic acid has the base sequence of bases 38538-54123 of SEQ ID NO.27, or has a base sequence which is identical to at least 70% of the base sequence of bases 38538-54123 of SEQ ID NO.27, and which functions to restore fertility. Still another method of the present invention comprises introducing a nucleic acid into rice, wherein the nucleic acid has the base sequence of bases 42357-53743, more preferably, bases 42132-48883 of SEQ ID NO.27, or has a base sequence which is identical to at least 70% of the base sequence of bases 42357-53743, more preferably, bases 42132-48883 of SEQ ID NO.27, and which functions to restore fertility. In an embodiment of the methods of the present invention, a base sequence which is identical to at least 70% of the base sequence of SEQ ID NO.27 or of the base sequence of bases 38538-54123 of SEQ ID NO.27 meets at least one of the following requirements 1) and 2):

1) a base corresponding to the base 45461 of SEQ ID NO.27 is A; and

2) a base corresponding to the base 49609 of SEQ ID NO.27 is A.

Another object of the present invention is to provide a method for discerning whether or not a subject rice individual or a seed thereof has the Rf-1 gene or not. The discerning method of the present invention utilizes a fact that a sequence determining the presence of the function of the rice restorer gene (the Rf-1 gene) positions between the polymorphism detection marker loci P4497 MboI and B56691 Xab I on rice chromosome 10.

In an embodiment of the methods of the present invention, the subject rice individual or the seed thereof is determined to have the Rf-1 gene, in the case that the nucleic acid having a base sequence which is identical to at least 70% of the base sequence of SEQ ID NO.27 or of the base sequence of bases 38538-54123 of SEQ ID NO.27, meets at least one of the following requirements 1) and 2):

1) a base corresponding to the base 45461 of SEQ ID NO.27 is A; and

2) a base corresponding to the base 49609 of SEQ ID NO.27 is A.

Another object of the present invention is to provide a method for inhibiting the function of the Rf-1 gene to restore fertility. The inhibition method of the present invention comprises, in an embodiment, introducing an antisense having at least 100 continuous bases in length, and having a base sequence complementary to a nucleic acid having the base sequence of SEQ ID NO.27, or to a nucleic acid having a base sequence which is identical to at least 70% of the base sequence of SEQ ID NO.27, and which functions to restore fertility. In another embodiment, the inhibition methods of the present invention comprise introducing an antisense having at least 100 continuous bases in length, and having a base sequence complementary to a nucleic acid having the base sequence of bases 38538-54123 of SEQ ID NO.27, or to a nucleic acid having a base sequence which is identical to at least 70% of the base sequence of bases 38538-54123 of SEQ ID NO.27, and which functions to restore fertility.

Another object of the present invention is to provide a nucleic acid having the base sequence of SEQ ID NO.27, or a nucleic acid having a base sequence which is identical to at least 70% of the base sequence of SEQ ID NO.27, and which functions to restore fertility. The present invention also provides a nucleic acid having the base sequence of bases 38538-54123 of SEQ ID NO.27, or a nuclei acid having a base sequence which is identical to at least 70% of the base sequence of bases 38538-54123 of SEQ ID NO.27, and which functions to restore fertility. The present invention also provides a nucleic acid having the base sequence of bases 42357-53743, more preferably, bases 42132-48883 of SEQ ID NO.27, or a nucleic acid having a base sequence which is identical to at least 70% of the base sequence of bases 42357-53743, more preferably, bases 42132-48883 of SEQ ID NO.27, and which functions to restore fertility.

### BRIEF DESCRIPTION OF THE DRAWING

FIG. 1 shows the results of chromosomal walking started from the RFLP marker locus S12564.

FIG. 2 shows an alignment of lambda clone contigs in relation to the BAC clone AC068923.

FIG. 3 shows the chromosomal organization of recombinant pollens proximal to the Rf-1 locus (all fertile) as mapped in close proximity to the Rf-1 locus based on the genotypes at the marker loci of 10 individuals (RS1, RS2, RC1-8) generated from the pollens. White bars represent japonica regions and black bars represent indica regions.

FIG. 4 is a gene map in which the locus of Rf-1 gene on chromosome 10 of rice is positioned on a linkage map in relation to various markers; the values of map distance were calculated from the segregation data from 1042 F1 individuals.

FIG. 5 shows fragments from 10 genomic clones used for the identification of the Rf-1 region by complementation assays. Lambda clones obtained by chromosomal walking (thin lines) were used for complementation assays of the chromosomal regions shown by bold lines. XSF18 was found to contain a deletion shown by dotted line.

FIG. 6 shows the results of complementation assays using a 15.7 kb fragment from XSG16 (Example 10) and a 16.2 kb fragment from XSF18 (Example 8). The plant transformed with the 15.7 kb fragment from XSG16 has restored fertility as proved by ears bowing.

### BEST MODES FOR PERFORMING THE INVENTION

We began by restricting the Rf-1 locus to a very small region on chromosome 10. On this basis, we developed PCR markers proximal to the Rf-1 locus and found a method for detecting the Rf-1 gene by utilizing on the linkage of these PCR markers to the Rf-1 locus. Specifically, the presence of the Rf-1 gene is tested and individuals homozygous for the Rf-1 gene are selected by genotyping at the novel PCR marker loci proximal to the Rf-1 locus on the basis that the Rf-1 locus is mapped between the PCR marker loci S12564 Tsp509I and C1361 MwoI on chromosome 10 of rice. We previously filed a patent application for the method for detecting the Rf-1 gene under Japanese Patent Application No. 2000-247204 on Aug. 17, 2000. The entire disclosure of the patent application is incorporated herein by reference.

I. Methods for Estimating the Genotype at the Rf-1 Locus Described in Japanese Patent Application No. 2000-247204

Japanese Patent Application No. 2000-247204 describes methods for determining whether or not a rice individual or seed under test has the Rf-1 gene on the basis that the Rf-1 locus is mapped between the PCR marker loci S12564 Tsp509I and C1361 MwoI on chromosome 10 of rice.

Markers

Primer pairs designed to be specific to particular regions near the locus of Rf-1 gene are used in PCR and the amplification products are treated with particular restriction enzymes; upon electrophoresis, rice of indica lines in some cases provide an observable band of a different size from that of rice of japonica lines. This band which is characteristic of indica lines is herein referred to as the Rf-1 linked band. Now that it has been made clear by the present inventors that the locus of Rf-1 gene is located between PCR markers S12564 Tsp509I and C1361 MwoI on chromosome 10 of rice, the skilled artisan can appropriately develop and employ PCR markers that are present in the neighborhood of Rf-1 gene.

For instance, according to the invention, a rice individual under test is checked to see if its genome contains at least one of the PCR markers listed below, thereby determining whether the individual under test has Rf-1 gene linked to those PCR markers:

(1) marker 1: PCR marker R1877 EcoRI which, when rice genomic DNA is subjected to PCR with DNA primers having the sequences of SEQ ID NO:1 and SEQ ID NO:2, can detect polymorphisms between rice individuals of the japonica and indica lines depending on whether the amplification products have a recognition site for restriction enzyme EcoRI;

(2) marker 2: PCR marker G4003 HindIII (SEQ ID NO:19) which, when rice genomic DNA is subjected to PCR with DNA primers having the sequences of SEQ ID NO:3 and SEQ ID NO:4, can detect polymorphisms between rice individuals of the japonica and indica lines depending on whether the amplification products have a recognition site for restriction enzyme HindIII;

(3) marker 3: PCR marker C1361 MwoI (SEQ ID NO:20) which, when rice genomic DNA is subjected to PCR employing DNA primers having the sequences of SEQ ID NO:5 and SEQ ID NO:6, can detect polymorphisms between rice individuals of the japonica and indica lines depending on whether the amplification products have a recognition site for restriction enzyme MwoI;

(4) marker 4: PCR marker G2155 MwoI (SEQ ID NO:21) which, when rice genomic DNA is subjected to PCR with DNA primers having the sequences of SEQ ID NO:7 and SEQ ID NO:8, can detect polymorphisms between rice individuals of the japonica and indica lines depending on whether the amplification products have a recognition site for restriction enzyme MwoI;

(5) marker 5: PCR marker G291 MspI (SEQ ID NO:22) which, when rice genomic DNA is subjected to PCR with DNA primers having the sequences of SEQ ID NO:9 and SEQ ID NO:10, can detect polymorphisms between rice individuals of the japonica and indica lines depending on whether the amplification products have a recognition site for restriction enzyme MspI;

(6) marker 6: PCR marker R2303 BslI (SEQ ID NO:23) which, when rice genomic DNA is subjected to PCR with DNA primers having the sequences of SEQ ID NO:11 and SEQ ID NO:12, can detect polymorphisms between rice individuals of the japonica and indica lines depending on whether the amplification products have a recognition site for restriction enzyme BslI;

(7) marker 7: PCR marker S10019 BstUI (SEQ ID NO:24) which, when rice genomic DNA is subjected to PCR with DNA primers having the sequences of SEQ ID NO:13 and SEQ ID NO:14, can detect polymorphisms between rice individuals of the japonica and indica lines depending on whether the amplification products have a recognition site for restriction enzyme BstUI;

(8) marker 8: PCR marker S10602 KpnI (SEQ ID NO:25) which, when rice genomic DNA is subjected to PCR with DNA primers having the sequences of SEQ ID NO:15 and SEQ ID NO:16, can detect polymorphisms between rice individuals of the japonica and indica lines depending on whether the amplification products have a recognition site for restriction enzyme KpnI; and

(9) marker 9: PCR marker S12564 Tsp509I (SEQ ID NO:26) which, when rice genomic DNA is subjected to PCR with DNA primers having the sequences of SEQ ID NO:17 and SEQ ID NO:18, can detect polymorphisms between rice individuals of the japonica and indica lines depending on whether the amplification products have a recognition site for restriction enzyme Tsp509I.

Assuming that the locus of Rf-1 gene was highly likely to be located near the nine RFLP marker regions R1877, G291, R2303, S12564, C1361, S10019, G4003, S10602 and G2155 on chromosome 10 of rice (see the results of RFLP linkage analysis described in Fukuta et al., 1992, Jpn. J. Breed. 42 (supl. 1) 164-165 and the RFLP linkage map of rice described in Harushima et al., 1998, Genetics, 148, 479-494), the present inventors converted those RFLP markers to co-dominant PCR markers such as CAPS markers or dCAPS markers as described below in Reference example 1 (Michaels and Amasino, 1998, The Plant Journal, 14(3), 381-385; Neff et al., 1998, The Plant Journal, 14(3), 387-392). As a result of this conversion, the PCR markers above have been obtained.

Among these PCR markers, one group consisting of PCR markers R1877 EcoRI, G291 MspI (SEQ ID NO:22), R2303 BslI (SEQ ID NO:23) and S12564 Tsp509I (SEQ ID NO:26) and the other group consisting of PCR markers C1361 MwoI (SEQ ID NO:20), S10019 BstUI (SEQ ID NO:24), G4003 HindIII (SEQ ID NO:19), S10602 KpnI (SEQ ID NO:25) and G2155 MwoI (SEQ ID NO:21) are on opposite sides of the locus of Rf-1 gene on chromosome 10 of rice.

Therefore, in one embodiment, the presence of the Rf-1 gene is detected by detecting Rf-1 linked bands by (a) at least one PCR marker selected from the group consisting of PCR markers R1877 EcoRI, G291 MspI, R2303 BslI and S12564 Tsp509I, and (b) at least one PCR marker selected from the group consisting of PCR markers C1361 MwoI, S10019 BstUI, G4003 HindIII, S10602 KpnI and G2155 MwoI. In this case, at least S12564 Tsp509I from group (a) and at least C1361 MwoI from group (b) are preferably used as the closest PCR markers to the Rf-1 gene. If Rf-1 linked bands are detected with PCR markers of both (a) and (b) in the genome of the rice under test, it can be estimated with a high probability that the rice contains Rf-1 gene.

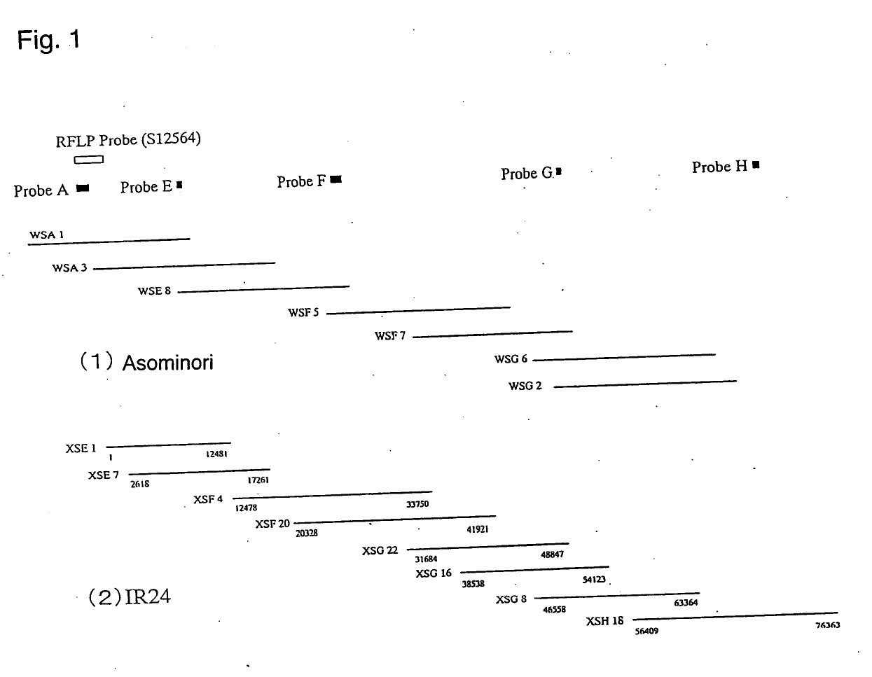

In another embodiment, Rf-1 linked bands are detected by at least two PCR markers of group (a) and at least two PCR markers of group (b) above. For example, a rice individual carrying the Rf-1 gene with a minimum of unwanted gene regions can be selected by picking up an individual in which Rf-1 linked bands are detected by markers of groups (a) and (b) more proximal to the Rf-1 gene but not detected by markers of groups (a) and (b) more distal from the Rf-1 gene on the gene map shown in FIG. 1. Again, it is preferred that at least one PCR marker of group (a) is S12564 Tsp509I and at least one PCR marker of group (b) is C1361 MwoI. Thus, the two PCR marker loci S12564 Tsp509I and C1361 MwoI are separated by a genetic distance of 0.3 cM. By utilizing this characteristic, the chromosomal region that is introduced from the Rf-1 gene donor parent can be narrowed down to a size of about 1 cM. This helps minimize the possibility of introducing into the restorer line a deleterious gene that may be present in the neighborhood of Rf-1 gene in the donor parent.

Detection of the Rf-1 Gene

In order to detect Rf-1 gene in the genome of a rice under test, any one of the above PCR markers is amplified from the genome of the rice by PCR using primers of SEQ ID NOS: 1-18 above and then detected by the polymerase chain reaction-restriction fragment length polymorphism method (PCR-RFLP). PCR-RFLP is a method that is applicable to the case where polymorphisms exist among variety lines at recognition sites of restriction enzymes in the sequences of PCR amplified DNA fragments and by which specific polymorphisms can conveniently be identified on the basis of cleavage patterns with those restriction enzymes (D. E. Harry et al., Theor. Appl. Genet. (1998), 97:327-336)

Restriction enzyme cleavage patterns show the bands as shown in Table 1 below on a visualized gel depending on the primer pair used.

TABLE 1

| | Approximate size |
|---|---|
| | (bp) of detected band |
| Detection of marker 1 (R1877 EcoRI) | |
| with primer pair 1 | |
| When the genome of test rice has | 1500 and 1700 |
| Rf-1 gene homozygously: | |
| When the genome of test rice has | 1500, 1700 and |
| Rf-1 gene heterozygously: | 3200 |
| When the genome of test rice has | 3200 |
| no Rf-1 gene: | |
| Detection of marker 2 (G4003 HindIII) | |
| with primer pair 2 | |
| When the genome of test rice has | 362 |
| Rf-1 gene homozygously: | |
| When the genome of test rice has | 95, 267 and 362 |
| Rf-1 gene heterozygously: | |
| When the genome of test rice has | 95 and 267 |
| no Rf-1 gene: | |
| Detection of marker 3 (C1361 MwoI) | |
| with primer pair 3 | |
| When the genome of test rice has | 50 and 107 |
| Rf-1 gene homozygously: | |
| When the genome of test rice has | 25, 50, 79 and 107 |
| Rf-1 gene heterozygously: | |
| When the genome of test rice has | 25, 50 and 79 |
| no Rf-1 gene: | |
| Detection of marker 4 (G2155 MwoI) | |
| with primer pair 4 | |
| When the genome of test rice has | 25, 27 and 78 |
| Rf-1 gene homozygously: | |
| When the genome of test rice has | 25, 27, 78 and 105 |
| Rf-1 gene heterozygously: | |
| When the genome of test rice has | 25 and 105 |
| no Rf-1 gene: | |
| Detection of marker 5 (G291 MspI) | |
| with primer pair 5 | |
| When the genome of test rice has | 25, 49 and 55 |
| Rf-1 gene homozygously: | |
| When the genome of test rice has | 25, 49, 55 and 104 |
| Rf-1 gene heterozygously: | |
| When the genome of test rice has | 25 and 104 |
| no Rf-1 gene: | |
| Detection of marker 6 (R2303 Bs1I) | |
| with primer pair 6 | |
| When the genome of test rice has | 238, 655 and 679 |
| Rf-1 gene homozygously: | |
| When the genome of test rice has | 238, 655, 679 and |
| Rf-1 gene heterozygously: | 1334 |
| When the genome of test rice has | 238 and 1334 |
| no Rf-1 gene: | |
| Detection of marker 7 (S10019 BstUI) | |
| with primer pair 7 | |
| When the genome of test rice has | 130, 218 and 244 |
| Rf-1 gene homozygously: | |
| When the genome of test rice has | 130, 218, 244 and |
| Rf-1 gene heterozygously: | 462 |
| When the genome of test rice has | 130 and 462 |
| no Rf-1 gene: | |
| Detection of marker 8 (S10602 KpnI) | |
| with primer pair 8 | |
| When the genome of test rice has | 724 |
| Rf-1 gene homozygously: | |
| When the genome of test rice has | 117, 607 and 724 |
| Rf-1 gene heterozygously: | |
| When the genome of test rice has | 117 and 607 |
| no Rf-1 gene: | |
| Detection of marker 9 (S12564 Tsp509I) | |
| with primer pair 9 | |
| When the genome of test rice has | 41 and 117 |
| Rf-1 gene homozygously: | |
| When the genome of test rice has | 26, 41, 91 and 117 |
| Rf-1 gene heterozygously: | |
| When the genome of test rice has | 26, 41 and 91 |
| no Rf-1 gene: | |

II. Identification of the Rf-1 Locus

As described above, Japanese Patent Application No. 2000-247204 discloses RFLP-PCR markers based on our finding that the Rf-1 locus is mapped between DNA marker loci S12564 Tsp509I and C1361 MwoI. Fertility-restoring lines are established by backcrossing the Rf-1 gene into a normal japonica variety not containing the Rf-1 gene. If the method for identifying the Rf-1 locus described in Japanese Patent Application No. 2000-247204 is used during this process, not only the restoring lines can be established efficiently (within 2-3 years) but also the length of insert fragments can be controlled.

However, introduction by crossing inevitably introduce regions proximal to Rf-1 at the same time. Japanese Patent Application No. 2000-247204 showed that the Rf-1 locus is mapped between DNA marker loci S12564 Tsp509I and C1361 MwoI, but the distance between both loci is about 0.3 cM, i.e. about 90 kbp. If a deleterious gene existed proximal to Rf-1, it would be undeniable that the deleterious gene might be inserted together with the Rf-1 gene.

Thus, we searched for regions linked to the Rf-1 gene between DNA marker loci S12564 Tsp509I and C1361 MwoI by chromosomal walking and genetic analysis based on the close linkage between the Rf-1 locus and the DNA marker locus S12564 Tsp509I. As a result, we successfully identified the region of the Rf-1 locus including the Rf-1 gene upto about 76 kb and determined the entire base sequence of said region. According to the present invention, it is possible to introduce the function of a fertility restorer gene into BT male sterile cytoplasms by genetic engineering techniques.

Specifically, in Japanese Patent Application No. 2000-247204, linkage analyses on a population of 1042 individuals prepared by pollinating MS Koshihikari with MS-FR Koshihikari (heterozygous at the Rf-1 locus) revealed one recombinant between the Rf-1 and S12564 Tsp509I loci and two recombinants between the Rf-1 and C1361 MwoI loci (Reference examples 1-2 herein). In the present invention, 4103 individuals were added to the population to analyze a total of 5145 individuals. As a result, one recombinant between the Rf-1 and S12564 Tsp509I loci and six recombinants between the Rf-1 and C1361 MwoI loci were newly found with a total of 2 and 8 recombinants. These 10 individuals were tested by the high-precision segregation analysis of the present invention as recombinants proximal to the Rf-1 locus (Example 1).

The frequency of 8 recombinants between the Rf-1 and C1361 MwoI loci as compared with 2 recombinants between the Rf-1 and S12564 Tsp509I loci means that the S12564 Tsp509I locus is genetically closer to the Rf-1 locus than the C1361 MwoI locus. Genetic distance (expressed in recombination frequency: cM) and physical distance (expressed in the number of base pairs: bp) are not always proportional to each other, but it can be normally expected that physical distance decreases with genetic distance.

Thus, we tried to isolate the Rf-1 locus by chromosomal walking started from the S12564 Tsp509I locus (Example 2). Chromosomal walking was performed on a genomic library prepared from λ DASH II vector using the genomic DNA of an indica variety IR24 and a japonica variety Asominori. IR24 is a variety carrying Rf-1, while Asominori is a variety not carrying Rf-1. As a result of chromosomal walking, contigs covering a chromosomal region of about 76 kb (ordered sets of overlapping clones on a chromosome) were able to be prepared from genomic clones of IR24, and the entire base sequence (76363 bp) thereof was determined.

Then, 12 markers were newly developed on the basis of the base sequence data or the like obtained and a high-precision segregation analysis was performed on the 10 recombinants proximal to Rf-1 locus described above (Example 3). As a result, a 65 kb sequence included in the chromosomal region of about 76 kb above was shown to contain a sequence determining the presence of the function of the Rf-1 gene. This region is covered by a contig consisting of 8 genomic clones. Each clone has a length of about 12-22 kb and has overlapping domains of at least 4.7 kb. Genes for rice are known to have a wide range of lengths (from short ones to large ones), but most of them seem to have a length of several kbs or less. Thus, at least one of these 8 genomic clones is expected to contain the full-length Rf-1 gene.

We further restricted the Rf-1 gene region in the chromosomal region of about 76 kb above and performed complementation assays to directly demonstrate the presence of a fertility restoring ability.

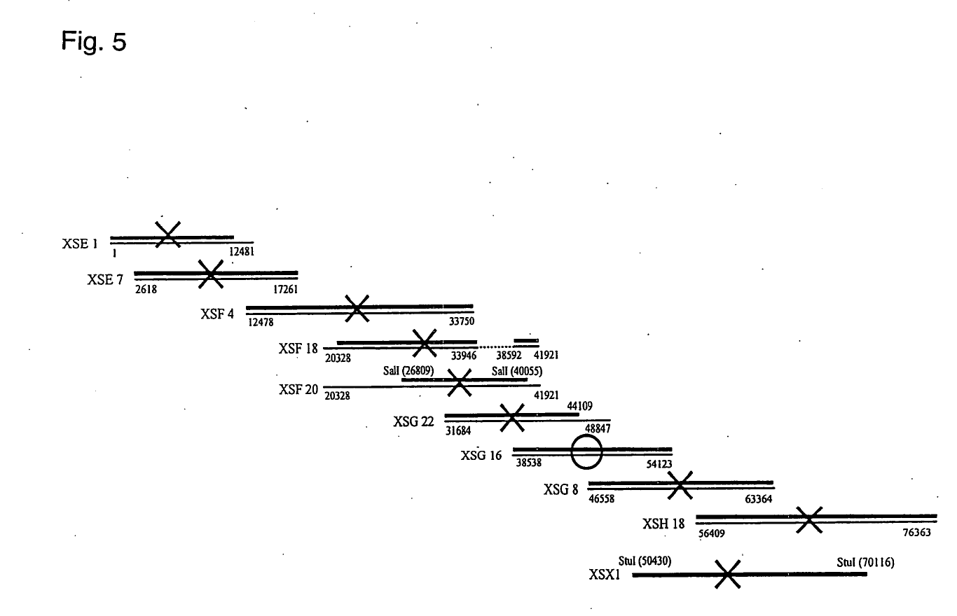

Specifically, 10 partial fragments (each 10-21 kb) in the above region of 76 kb were separately introduced into immature seeds of a male sterility line MS Koshihikari by genetic engineering techniques (FIG. 5). Of the 10 partial fragments used, 8 fragments are derived from 8 genomic clones previously obtained by chromosomal walking (XSE1, XSE7, XSF4, XSF20, XSG22, XSG16, XSG8 and XSH18 shown in FIG. 1 and described in Example 3). Additionally, fragments derived from 2 clones XSF18 and XSX1 were also analyzed by complementation assays. XSF18 is identical to XSF20 at the 5′ and 3′ ends (bases 20328 and 41921 of SEQ ID NO: 27, respectively), but lacks internal bases 33947-38591. This is because clone XSF18 was initially isolated but found to contain the above deletion during amplification after isolation, and therefore, the amplification step was freshly taken to isolate a complete clone designated XSF20 (Example 8). XSX1 is a clone freshly prepared from clones XSG8 and XSH18 by restriction enzyme treatment and ligation to contain sufficient overlapping domains because of the overlapping domains of both clones are relatively small (about 7 kb) (Example 13).

If the insert fragment completely contains the Rf-1 gene, transformed individuals at this generation restore fertility because Rf-1 is a dominant gene. In complementation assays plants transformed with each fragment were evaluated for seed fertility to find that those transformed with a 15.6 kb fragment (including bases 38538-54123 of SEQ ID NO: 27) derived from the λ phage clone XSG16 restored seed fertility (Example 10). Plants transformed with the other fragments were all sterile. These results showed that the above 15.6 kb fragment completely contains the Rf-1 gene. Moreover, a method for introducing the Rf-1 gene by genetic engineering techniques was provided by the present invention and demonstrated to be effective.

To further specify the region of the λ phage clone XSG16 in which the Rf-1 gene is contained, we evaluated seed fertility of shorter fragments than the 15.6 kb fragment (including bases 38538-54123 of SEQ ID NO: 27) by complementation assays. As a result, plants transformed with a 11.4 kb fragment derived from XSG16 (including bases 42357-53743 of SEQ ID NO: 27) were shown to restore seed fertility (Example 10(2)). Plants transformed with a further shorter 6.8 kb fragment (including bases 42132-48883 of SEQ ID NO: 27) also restored seed fertility (Example 10(3)). These results showed that the above 6.8 kb fragment contains the Rf-1 gene.

III. Nucleic Acids Containing the Rf-1 Locus

The present invention provides nucleic acids containing the locus of a fertility restorer gene (Rf-1). The nucleic acids containing the locus of a fertility restorer gene (Rf-1) of the present invention include a nucleic acid having the base sequence of SEQ ID NO.27, or a nucleic acid having a base sequence which is identical to at least 70% of the base sequence of SEQ ID NO.27, and which functions to restore fertility.

As described in Example 10, it was confirmed that the Rf-1 gene is completely contained in especially bases 38538-54123 of the base sequence of SEQ ID NO: 27. Accordingly, the present invention especially provides a nucleic acid having the base sequence of bases 38538-54123 of SEQ ID NO.27, or has a base sequence which is identical to at least 70% of the base sequence of bases 38538-54123 of SEQ ID NO.27, and which functions to restore fertility. As used herein, the term “the base sequence of SEQ ID NO: 27” refers to the entire sequence of SEQ ID NO: 27 or a part thereof participating in fertility restoring function, especially bases 38538-54123 according to the context. More preferably, it refers to bases 42357-53743, still more preferably bases 42132-48883.

In the examples below, a nucleic acid was isolated from a genomic library of indica rice IR24 containing the Rf-1 gene as a nucleic acid containing a fertility restorer gene (Rf-1) and determined to have the base sequence of SEQ ID NO: 27. However, the nucleic acid containing a fertility restorer gene (Rf-1) of the present invention can be derived from any indica variety carrying the RF-1 gene. The indica varieties carrying the Rf-1 gene include, but not specifically limited to, e.g. IR24, IR8, IR36, IR64, Chinsurah and BoroII. Known japonica varieties not carrying the Rf-1 gene include, but not limited to, Asominori, Koshihikari, Kirara 397, Akihikari, Akitakomachi, Sasanishiki, Kinuhikari, Nipponbare, Hatsuboshi, Koganebare, Hinohikari, Mineasahi, Aichinokaori, Hatsushimo, Akebono, Fujihikari, Minenoyukimochi, Kokonoemochi, Fukuhibiki, Dontokoi, Gohyakumangoku, Hanaechizen, Todorokiwase, Haenuki, Domannaka, Yamakikari, etc. The “indica” and “japonica” varieties are well known to those skilled in the art and the rice varieties encompassed by the present invention can be readily determined by those skilled in the art.

Nucleic acids of the present invention include DNA in both single-stranded and double-stranded forms, as well as the RNA complement thereof. DNA includes, for example, genomic DNA (including corresponding cDNA), chemically synthesized DNA, DNA amplified by PCR, and combinations thereof.

Nucleic acids containing the Rf-1 gene of the present invention preferably have the base sequence of SEQ ID NO: 27. More than one codon may encode the same amino acid, and this is called degeneracy of the genetic code. Thus, a DNA sequence not completely identical to SEQ ID NO: 27 may encode a protein having an amino acid sequence completely identical to SEQ ID NO: 27. Such a variant DNA sequence may result from silent mutation (e.g., occurring during PCR amplification), or can be a product of deliberate mutagenesis of a native sequence.

It is well known for those skilled in the art that even proteins having the same function may have different amino acid sequences depending on the varieties from which they are derived. The Rf-1 gene of the present invention includes such homologs and variants of the base sequence of SEQ ID NO: 27 so far as they functions to restore fertility. The expression “function to restore fertility” means that fertility is conferred on a rice individual or seed when such a DNA fragment is introduced. Fertility restoration may result from the expression of a protein by the Rf-1 gene or some function of the nucleic acid (DNA or RNA) per se of the Rf-1 gene in conferring fertility.

Whether or not a homolog or variant of the Rf-1 gene functions to restore fertility can be examined by, but not limited to, the following method, for example. A nucleic acid fragment under test is introduced into immature seeds obtained by pollinating MS Koshihikari (sterile line) with MS-FR Koshihikari according to the method of Hiei et al. (Plant Journal (1994), 6(2), p. 272-282). As the resulting transformants are cultured under normal conditions, the seeds mature only when the nucleic acid fragment under test functions to restore fertility.

The nucleic acid derived from a corresponding region of japonica Asominori not carrying the Rf-1 gene has the base sequence shown in SEQ ID NO: 28. Corresponding parts of SEQ ID NO: 28 and SEQ ID NO: 27 have an overall identity of about 98%. Thus, nucleic acids containing the locus of a fertility restorer gene (Rf-1) of the present invention are at least about 70%, preferably about 80% or more, more preferably 90% or more, still more preferably 95% or more, most preferably 98 or more % identical to SEQ ID NO: 27.

The percent identity may be determined by visual inspection and mathematical calculation. Alternatively, the percent identity of two nucleic acid sequences can be determined by comparing sequence information using the GAP computer program, version 6.0 described by Devereux et al., Nucl. Acids Res., 12:387 (1984) and available from the University of Wisconsin Genetics Computer Group (UWGCG). The preferred default parameters for the GAP program include: (1) a unary comparison matrix (containing a value of 1 for identities and 0 for non-identities) for bases, and the weighted comparison matrix of Gribskov and Burgess, Nucl. Acids Res., 14:6745 (1986), as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps. Other programs used by those skilled in the art of sequence comparison may also be used.

Nucleic acids of the present invention also include nucleic acids which are capable of hybridizing to the base sequence of SEQ ID NO: 27 under conditions of moderately stringent conditions and functions to restore fertility, and nucleic acids which are capable of hybridizing to the base sequence of SEQ ID NO: 27 under conditions of highly stringent conditions and functions to restore fertility.

As used herein, conditions of moderate stringency can be readily determined by those having ordinary skill in the art based on, for example, the length of the DNA. The basic conditions are set forth by Sambrook et al. Molecular Cloning: A Laboratory Manual, 2nd. Vol. 1, pp. 1.101-104, Cold Spring Harbor Laboratory Press, (1989), and include use of a prewashing solution for the nitrocellulose filters 5×SSC, 0.5% SDS, 1.0 mM EDTA (pH 8.0), hybridization conditions of about 1×SSC to 6×SSC at about 40° C. to 60° C. (or other similar hybridization solution, such as Stark's solution, in about 50% formamide at about 42° C.), and washing conditions of about 60° C., 0.5×SSC, 0.1% SDS. The hybridization temperature is about 15-20° C. lower when the hybridization solution contains about 50% formamide. Conditions of high stringency can also be readily determined by the skilled artisan based on, for example, the length of the DNA. Generally, conditions of high stringency include hybridization and/or washing conditions at higher temperatures and/or lower salt concentrations than in the conditions of moderate stringency described above. For example, such conditions include hybridization conditions of 0.1×SSC to 0.2×SSC at about 60-65° C. and/or washing conditions of 0.2×SSC, 0.1% SDS at about 65-68° C. The skilled artisan will recognize that the temperature and wash solution salt concentration can be adjusted as necessary according to factors such as the length of the probe.

DNAs of the present invention also include nucleic acids that differ from the base sequence of SEQ ID NO: 27 due to deletions, insertions or substitutions of one or more bases while retaining a fertility restoring function. So far as a fertility restoring function is retained, the number of bases to be deleted, inserted or substituted is not specifically limited, but preferably 1 to several thousands, more preferably 1-1000, still more preferably 1-500, even more preferably 1-200, most preferably 1-100.

Once the Rf-1 gene is further specified on the basis of the descriptions herein, it can be used by those skilled in the art after nucleic acids such as other regions than the Rf-1 gene or intron regions in the Rf-1 gene are removed. A given amino acid may be replaced, for example, by a residue having similar physiochemical characteristics. Examples of such conservative substitutions include changes from one aliphatic residue to another, such as changes from one to another of Ile, Val, Leu, or Ala; changes from one polar residue to another, such as changes between Lys and Arg, Glu and Asp, or Gln and Asn; or changes from one aromatic residue to another, such as changes from one to another of Phe, Trp, or Tyr. Other well-known conservative substitutions include e.g. changes between entire regions having similar hydrophobic characteristics. Those skilled in the art can introduce desired deletions, insertions or substitutions by well-known gene engineering techniques using e.g. site-specific mutagenesis as described in Sambrook et al., Molecular Cloning: A Laboratory Manual, 2nd edition, Cold Spring Harbor Laboratory Press, (1989).

We compared an indica variety IR24 carrying the Rf-1 gene (SEQ ID NO: 27) with japonica varieties not carrying it such as Asominori (SEQ ID NO: 28) and a Nipponbare BAC clone deposited with GenBank (Accession No. AC068923). As a result, we found that the Rf-1 region of the indica variety containing the Rf-1 gene has at least the following single bases polymorphisms (SNP).

1) a base corresponding to the base 1239 of SEQ ID NO: 27 is A;

2) a base corresponding to the base 6227 of SEQ ID NO: 27 is A;

3) a base corresponding to the base 20680 of SEQ ID NO: 27 is G;

4) a base corresponding to the base 45461 of SEQ ID NO: 27 is A;

5) a base corresponding to the base 49609 of SEQ ID NO: 27 is A;

6) a base corresponding to the base 56368 of SEQ ID NO: 27 is T;

7) a base corresponding to the base 57629 of SEQ ID NO: 27 is C; and

8) a base corresponding to the base 66267 of SEQ ID NO: 27 is G.

Thus, nucleic acids containing the Rf-1 region of the present invention preferably meet one to all of the requirements 1)-8) above.

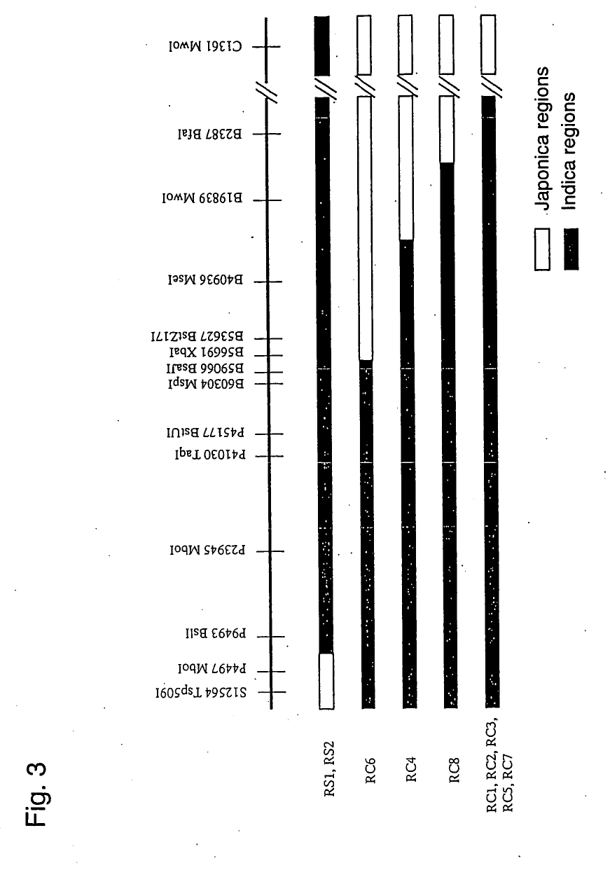

In Example 3 below, the chromosomal organizations of recombinants proximal to the Rf-1 gene (RS1-RS2, RC1-RC8) were tested in the Rf-1 region. The results showed that a sequence determining the presence of the function of the Rf-1 gene is contained in the base sequence of bases 1239-66267 of SEQ ID NO: 27, i.e. in a region from the P4497 MboI to B56691 XbaI loci (about 65 kb) as estimated at maximum (FIG. 3). However, there is a possibility that it is important for the expression of the genetic function of the Rf-1 gene that the Rf-1 gene is partially of the indica genotype, and that the genetic function may not be significantly changed whether the remaining regions are of the japonica or indica genotype. There may be an extreme case that the coding region is completely identical and only the promoter region is different between japonica and indica, and that the promoter region and the coding region are only partially included in the region from P4497 the MboI to B56691 XbaI loci (about 65 kb). Therefore, it cannot be concluded that the common indica region above (bases 1239-66267 of SEQ ID NO: 27) completely contains the entire Rf-1 gene. However, it is thought that at least SEQ ID NO: 27 completely contains the entire Rf-1 gene for the following reasons:

1) the size of a gene is normally several kilobases, and rarely exceeds 10 kb;

2) the genomic base sequence of IR24 determined by the present invention (SEQ ID NO: 27) completely contains the common indica region above;

3) the 5′ end of SEQ ID NO: 27 is located 1238 bp upstream of the 5′ end of the common indica region above and forms a part of another gene (S12564); and

4) the 3′ end of SEQ ID NO: 27 is located 10096 bp downstream of the 3′ end of the common indica region above.

In this way, we first succeeded in restricting the region of the Rf-1 gene to 76 kb. Thus, nucleic acids containing the region of the Rf-1 gene of the present invention are extremely less likely to contain other genes proximal to the Rf-1 gene as compared with those selected with the co-dominant marker locus at a genetic distance of about 1 cM (about 300 kb) from the Rf-1 gene described in a prior documents such as Japanese Patent Public Disclosure No. 2000-139465. Moreover, they are less likely to contain other genes than those selected with the DNA marker loci S12564 Tsp509I and C1361 MwoI (at a distance of about 0.3 cM between them) described in our prior Japanese Patent Application No. 2000-247204.

We further confirmed by complementation assays that the Rf-1 gene is completely contained in especially bases 38538-54123 of the base sequence of SEQ ID NO: 27. In an embodiment of the present invention, therefore, the base sequence at least 70% identical to the base sequence of SEQ ID NO: 27 or to the base sequence of bases 38538-54123 of SEQ ID NO: 27 meets at least one of the following requirements 1) and 2):

1) a base corresponding to the base 45461 of SEQ ID NO: 27 is A;

2) a base corresponding to the base 49609 of SEQ ID NO: 27 is A.

IV. Method for Restoring Rice Fertility

The present invention provides a method for restoring rice fertility comprising introducing a nucleic acid into rice, wherein the nucleic acid has the base sequence of SEQ ID NO.27, or has a base sequence which is identical to at least 70% of the base sequence of SEQ ID NO.27, and which functions to restore fertility. The methods of the present invention may comprise introducing a nucleic acid into rice, wherein the nuclei acid has a portion of SEQ ID NO: 27, especially bases 38538-54123, preferably bases 42357-53743, more preferably bases 42132-48883 of SEQ ID NO: 27 or has a base sequence which is identical to at least 70% identical of the base sequence of bases 38538-54123, preferably bases 42357-53743, more preferably bases 42132-48883 of SEQ ID NO: 27 and, which functions to restore fertility.

In the present invention, the nucleic acid containing the locus of a fertility restorer gene (Rf-1) that can be introduced into rice can be any one of the nucleic acids described above in “III. Nucleic acids containing the Rf-1 locus”. The method for introducing the nucleic acid into rice is not specifically limited but can be any known method. Nucleic acids of the present invention can be introduced by known genetic engineering techniques or crossing. Genetic engineering techniques are preferably used because inclusion of other neighboring genes can be prevented and the period for establishing a line can be shortened.

Any suitable expression system for transduction by genetic engineering techniques can be employed. Recombinant expression vectors comprise a nucleic acid containing a fertility restorer gene (Rf-1) of the invention that can be introduced into rice, operably linked to suitable transcriptional or translational regulatory base sequences, such as those derived from a mammalian, microbial, viral, or insect gene.

Examples of regulatory sequences include transcriptional promoters, operators, or enhancers, an mRNA ribosomal binding site, and appropriate sequences which control transcription and translation initiation and termination. Base sequences are operably linked to a regulatory sequence when the regulatory sequence is functionally associated with the DNA sequences. Thus, a promoter base sequence is operably linked to a DNA sequence if the promoter base sequence controls the transcription of the DNA sequence. An origin of replication that confers the ability to replicate in rice, and a selection gene by which transformants are identified, are generally incorporated into expression vectors. As for selectable markers, those commonly used can be used by standard methods. Examples are genes resistant to antibiotics such as tetracycline, ampicillin, kanamycin, neomycin, hygromycin or spectinomycin.

In addition, a sequence encoding an appropriate signal peptide (native or heterologous) can be incorporated into expression vectors. A DNA sequence for a signal peptide (secretory leader) may be fused in frame to a nucleic acid sequence of the invention so that the DNA is initially transcribed, and the mRNA translated into a fusion protein containing the signal peptide.

The present invention also provides recombinant vectors containing a gene of the present invention. Methods for integrating a DNA fragment of a gene of the present invention into a vector such as a plasmid are described in e.g. Sambrook, J. et al, Molecular Cloning, A Laboratory Manual (2nd edition), Cold Spring Harbor Laboratory, 1.53 (1989). Commercially available ligation kits (e.g. available from TAKARA) can be conveniently used. Thus obtained recombinant vectors (e.g. recombinant plasmids) are transferred into host rice cells.

Vectors can be conveniently prepared by linking a desired gene to a recombinant vector available in the art (e.g. plasmid DNA) by standard methods. Plant transforming vectors are especially useful for conferring fertility on rice using a nucleic acid fragment of the present invention. Vectors for plants are not specifically limited so far as they can express the gene of interest in plant cells to produce the protein, but preferably include pBI221, pBI121 (Clontech), and vectors derived therefrom. Especially, examples of vectors for transforming rice belonging to monocotyledons include pIG121Hm and pTOK233 (Hiei et al., Plant J., 6, 271-282 (1994)), and pSB424 (Komari et al., Plant J., 10, 165-174 (1996)).

Transgenic plants can be prepared by replacing the β-glucuronidase (GUS) gene in the above vectors with a nucleic acid fragment of the present invention to construct a plant transforming vector and transfecting it into a plant. The plant transforming vector preferably comprises at least a promoter, a start codon, a desired gene (a nucleic acid sequence of the present invention or a part thereof), a stop codon and a terminator. It may also contain a DNA encoding a signal peptide, an enhancer sequence, non-translated 5′ and 3′ regions of the desired gene, a selectable marker region, etc., as appropriate. Promoters and terminators are not specifically limited so far as they are functional in plant cells, among which constitutive expression promoters include the 35S promoter initially contained in the above vectors as well as promoters for actin and ubiquitin genes.

Suitable methods for introducing a plasmid into a host cell include the use of calcium phosphate or calcium chloride/rubidium chloride, electroporation, electroinjection, chemical treatment with PEG or the like, the use of a gene gun described in Sambrook, J. et al., Molecular Cloning, A Laboratory Manual (2nd edition), Cold Spring Harbor Laboratory, 1.74 (1989). Plant cells can be transformed by e.g. the leaf disc method [Science, 227, 129 (1985)] or electroporation [Nature, 319, 791 (1986)].

Methods for transferring a gene into a plant include the use of Agrobacterium (Horsch et al., Science, 227, 129 (1985); Hiei et al., Plant J., 6, 271-282 (1994)), electroporation (Fromm et al., Nature, 319, 791 (1986)), PEG (Paszkowski et al., EMBO J., 3, 2717 (1984)), microinjection (Crossway et al., Mol. Gen. Genet., 202, 179 (1986)), particle bombardment (McCabe et al., Bio/Technology, 6, 923 (1988)). Methods are not specifically limited so far as they are suitable for transfecting a nucleic acid into a desired plant.

Transduction by crossing can be performed as follows, for example. First, F₁ obtained by crossing an Rf-1 donor parent and a japonica variety is backcrossed with the japonica variety. The resulting individuals are screened for those homozygous for japonica at the S12564 Tsp509I locus and heterozygous at the P4497 MboI and B53627 BstZ17I loci and further backcrossed. The resulting individuals are screened for those heterozygous at the P4497 MboI and B56691 XbaI loci and homozygous for japonica at the B53627 BstZ17I locus and further backcrossed. Subsequently, about 10 cycles of screening each backcrossed generation for individuals heterozygous at the P4497 MboI and B56691 XbaI loci and subjecting them to the subsequent backcrossing are repeated. Finally, individuals heterozygous at the P4497 MboI and B56691 XbaI loci are self-fertilized and the resulting individuals are screened for those homozygous for indica at both loci, whereby a restorer line inheriting a limited chromosomal region from the P4497 MboI to B56691 XbaI loci from the Rf-1 donor parent can be obtained.

According to the present invention, nucleic acids containing a fertility restorer gene (Rf-1) were isolated, whereby the Rf-1 gene can be introduced into a rice variety using genetic engineering techniques to establish a restorer line. The present invention succeeded in restricting the Rf-1 region to 76 kb or less. Therefore, nucleic acids containing the Rf-1 locus of the present invention are extremely less likely to contain other genes neighboring the Rf-1 gene than those of the prior art. Moreover, the entire base sequence of the region containing the Rf-1 gene was determined by the present invention. Those skilled in the art can proceed with analysis of the Rf-1 gene itself on the basis of the description herein. Thus, only the Rf-1 gene can be introduced without including any neighboring gene. This is especially important when neighboring genes bring deleterious traits. Furthermore, restorer lines can be established in a shorter period such as 1-2 years than obtained by crossing.

In complementation assays described in Examples 4-13 herein, MS Koshihikari (having BT cytoplasm and a core gene substantially identical to Koshihikari) was actually transformed by an Agrobacterium-mediated method using fragments from 10 clones described in FIG. 5. The results demonstrated that fertility restorer lines are established from a nucleic acid containing the base sequence of bases 38538-54123, preferably bases 42357-53743, more preferably bases 42132-48883 of SEQ ID NO: 27.

Agrobacterium-mediated methods for establishing rice restorer lines are described in, but not limited to, Hiei et al., Plant J., 6, pp. 271-282 (1994), Komari et al., Plant J., 10, p. 165-174 (1996), Ditta et al., Proc. Natl. Acad. Sci. USA 77: pp. 7347-7351 (1980), etc.

First, a plasmid vector containing a nucleic acid fragment of interest to be inserted is prepared. Suitable plasmid vectors include e.g. pSB11, pSB22 and the like having a plasmid map described in Komari et al., Plant J., 10, pp. 165-174 (1996), supra. Alternatively, those skilled in the art can also construct an appropriate vector by themselves on the basis of plasmid vectors such as pSB11, pSB22 described above. In the examples herein below, an intermediate vector pSB200 having a hygromycin-resistant gene cassette was prepared on the basis of pSB11, and used. Specifically, a nopaline synthase terminator (Tnos) was first fused to a ubiquitin promoter and a ubiquitin intron (Pubi-ubiI). A hygromycin-resistant gene (HYG(R)) was inserted between ubiI and Tnos of the resulting Pubi-ubiI-Tnos complex to give a Pubi-ubiI-HYG(R)-Tnos assembly. This assembly was fused to a HindIII/EcoRI fragment of pSB11 (Komari et al., supra.) to give pKY205. Linker sequences for adding restriction enzyme sites NotI, NspV, EcoRV, KpnI, SacI, EcoRI were inserted into the Hind III site upstream of Pubi of this pKY205 to give pSB200 having a hygromycin-resistant gene cassette.

Then, E. coli cells (e.g. DH5a, JM109, MV1184, all commercially available from e.g. TAKARA) are transformed with the recombinant vector containing the nucleic acid inserted.

Thus transformed E. coli cells are used for triparental mating with an Agrobacterium strain preferably in combination with a helper E. coli strain according to e.g. the method of Ditta et al. (1980). Suitable Agrobacterium strains include Agrobacterium tumefaciens strains such as LBA4404/pSB1, LBA4404/pNB1, LBA4404/pSB3, etc. They all have a plasmid map described in Komari et al., Plant J., 10, pp. 165-174 (1996), supra. and can be used by those skilled in the art by constructing a vector by themselves. Suitable helper E. coli strains include, but not limited to, e.g. HB101/pRK2013 (available from Clontech). A report shows that E. coli cells carrying pRK2073 can also be used as helper E. coli though they are less common (Lemas et al., Plasmid 1992, 27, pp. 161-163).

Then, the Agrobacterium cells mated as intended are transformed into male sterility rice according to e.g. the method of Hiei et al (1994). Necessary immature rice seeds for transformation can be prepared by e.g. pollinating male sterility rice with a japonica variety.

Fertility restoration in transformed plants can be assessed by e.g. evaluating seed fertility in standing plants about one month after heading. Evaluation on standing plants means observation of plants grown in a field or the like. An alternative method is a laboratory study of grain ripening percentages in the ear.

V. Methods for Discerning the Presence of the Rf-1 Gene

According to the present invention, it was shown that a sequence determining the presence of the function of the Rf-1 gene is located between the polymorphism-detecting marker loci P4497 MboI and B56691 XbaI on rice chromosome 10. Moreover, complementation assays confirmed that the Rf-1 gene is completely contained in especially bases 38538-54123 of the base sequence of SEQ ID NO: 27.

Comparison of the base sequence of an indica variety carrying the Rf-1 gene (IR24) (SEQ ID NO: 27) with those of japonica varieties not carrying said gene (Asominori (SEQ ID NO: 28) and Nipponbare BAC clone AC068923) revealed the presence of polymorphisms between both varieties. As a result, it became possible to conveniently, rapidly and exactly discern whether or not a rice plant or seed under test carries the Rf-1 gene on the basis of polymorphisms in base sequence in regions neighboring the Rf-1 gene.

Therefore, the present invention also provides a method for discerning whether or not a subject rice individual or a seed thereof has the Rf-1 gene or not, wherein the method utilizing a fact that a sequence determining the presence of the function of the Rf-1 gene positions between the polymorphism detection marker loci P4497 MboI and B56691 Xba I on rice chromosome 10.

Polymorphisms can be detected by any known method. For example, known methods include assays for restriction fragment length polymorphisms (RFLPs); direct determination by sequencing; cutting a genomic DNA with a 8-base recognizing restriction enzyme, and then radioactivelly labeling the ends and further cutting the labeled digest with 6-base and 4-bases recognizing restriction enzyme and then developing the digest by two-dimensional electrophoresis (RLGS, Restriction Landmark Genome Scanning); etc. AFLP analysis (amplified fragment length polymorphism; P. Vos et al., Nucleic Acids Res. Vol. 23, pp. 4407-4414 (1995)) has also been developed wherein RFLP is amplified/detected by polymerase chain reaction (PCR).

For example, conventional methods involved detecting RFLPs via PCR amplification (conversion of RFLP markers into PCR markers) or detecting polymorphisms in microsatellites via PCR amplification (microsatellite markers) as illustrated below.

Conversion of RFLP Markers into PCR Markers

A. PCR markers based on polymorphisms in genomic regions corresponding to RFLP probes (D. E. Harry, B. Temesgen, D. B. Neale; Codominant PCR-based markers for Pinus taeda developed from mapped cDNA clones, Theor. Appl. Genet. (1998) 97: pp. 327-336). After performing genomic PCR using primers designed for an RFLP marker probe sequence (“RFLP” is a polymorphism observed by Southern analysis using a DNA fragment as a probe. The base sequence of the DNA fragment used as a probe is called “RFLP marker probe sequence”), a PCR marker can be prepared by either of the following two procedures. A first procedure involves treating the products with a series of restriction enzymes to search for a restriction enzyme causing a fragment length polymorphism, and a second procedure involves searching for a polymorphism by varietal comparison of the base sequences of the products and preparing a PCR marker based on the polymorphism.

B. PCR markers based on identification of RFLP-causing sites. A PCR marker can be obtained by identifying an RFLP-causing site (a restriction enzyme recognition site carried by only one of two varieties compared) present in or near (normally within several kbs) an RFLP marker probe sequence.

Microsatellite Markers

Microsatellites are repeat sequences of about 2 to 4 bases such as (CA)ₙ that are present in great numbers in genomes. If a varietal polymorphism occurs in repetition number, a polymorphism can be observed in PCR product length by PCR using primers designed in adjacent regions, whereby the DNA polymorphism can be detected. Markers for detecting polymorphisms using microsatellites are called microsatellite markers (O. Parnaud, X. Chen, S. R. McCouch, Mol. Gen. Genet. (1996) 252: pp. 597-607).

Methods for detecting polymorphisms in the present invention are not specifically limited. From the viewpoint of efficiency and convenience, PCR-RFLP is preferred, which is a combination of PCR and RFLP to identify polymorphisms from their restriction enzyme cleavage patterns in cases where they exist among variety lines at restriction enzyme recognition sites in the sequences of DNA fragments amplified by PCR. PCR-RFLP is also called CAPS (cleaved amplified polymorphic sequence). If any suitable restriction enzyme recognition site is not present in a region showing polymorphisms, a modified CAPS called dCAPS (derived cleaved amplified polymorphic sequence) can also be used wherein restriction enzyme sites are introduced during PCR (Michaels, S. D. and Amasino, R. M. (1998), The Plant Journal 14(3) 381-385; A. Konieczny et al., (1993), Plant J. 4(2) pp. 403-410; Neff, M. M., Neff, J. D., Chory, J. and Pepper, A. E. (1998), The Plant Journal 14(3) 387-392). These methods are explained in more detail below.

CAPS, dCAPS

The method for discerning of the present invention comprise, but not limited to:

i) preparing a pair of primers based on the base sequences of a site showing a polymorphism in the base sequences between indica and japonica varieties at the Rf-1 locus and its adjacent regions to amplify said base sequences;

ii) performing nucleic acid amplification reaction(s) using the genomic DNA of the subject rice individual or the seed thereof as a template; and

iii) discerning whether or not the subject rice individual or the seed thereof has the Rf-1 gene based on the polymorphism found in the nucleic acid amplification product.

The step of preparing a primer pair in i) preferably comprises any of the following means:

a) when a change containing a deleted region exists in the polymorphism in the nucleic acid amplification product, preparing a pair of primers for nucleic acid amplification to flank the deleted region to form a marker for detecting the polymorphism;

b) when a base change causing a difference in restriction enzyme recognition exists in the polymorphism in the nucleic acid amplification product, preparing a pair of primers for nucleic acid amplification to flank the base change site to form a marker for detecting the polymorphism; or

c) when a base change causing no difference in restriction enzyme recognition exists in the polymorphism in the nucleic acid amplification product, preparing a pair of primers for introducing a mismatch, wherein pair of primers contain the base change site and alters a region containing the base change site into a base sequence causing a difference in restriction enzyme recognition in the nucleic acid amplification product to form a marker for detecting the polymorphism.

Suitable polymorphic sites for discerning the presence of the Rf-1 gene in the present invention can be appropriately selected so that a polymorphism detecting marker can be prepared as described below on the basis of comparison of, but not limited to, the base sequence of an indica variety carrying the Rf-1 gene (IR24) (SEQ ID NO: 27) with those of japonica varieties not carrying said gene (Asominori (SEQ ID NO: 28) and Nipponbare BAC clone AC068923).

If the polymorphism found causes a difference in restriction enzyme recognition, for example, a pair of primers for nucleic acid amplification are prepared to flank the polymorphic site and used for detecting the polymorphism. Primers are preferably designed not to be specific for highly repeated sequences to avoid undesired products. If the polymorphism found does not cause a difference in restriction enzyme recognition, markers can be prepared by applying the dCAPS method described above. Primers for dCAPS markers are preferably designed not to be specific for repeat sequences and to provide a product length of preferably 50-300 bases, more preferably about 100 bases to ease identification of polymorphisms.

If the polymorphism found involves a microsatellite, nucleic acid amplification primers are prepared to flank the microsatellite and used to detect the polymorphism. Again, the primers are preferably designed not to be specific for repeat sequences.

1) Nucleic Acid Amplification

In the present invention, a pair of primers are preferably prepared for amplifying adjacent regions containing polymorphisms on the basis of the determined base sequence of the nucleic acid of a subject rice individual or seed at the Rf-1 locus. The primer pair is used to perform a nucleic acid amplification reaction with the genomic DNA of the subject rice individual or seed as a template. The nucleic acid amplification reaction is preferably polymerase chain reaction (PCR) (Saiki et al., 1985, Science 230, pp. 1350-1354).

The pair of primers for nucleic acid amplification can be prepared by any known method on the basis of the base sequence of a polymorphic site and adjacent regions thereto. Specifically, a primer pair can be prepared on the basis of the base sequence of a polymorphic site and adjacent regions thereto by a process comprising generating a single-stranded DNA having the same base sequence as the base sequence of the polymorphic site and adjacent regions thereto or a base sequence complementary to said regions or, if necessary, generating the single-stranded DNA containing a modification without affecting the binding specificity to the base sequence of the polymorphic site and adjacent regions thereto provided that the following conditions are satisfied:

1) the length of each primer should be 15-30 bases;

2) the proportion of G+C in the base sequence of each primer should be 30-70%;

3) the distribution of A, T, G and C in the base sequence of each primer should not be partially largely uneven;

4) the length of the nucleic acid amplification product amplified by the primer pair should be 50-3000 bases, preferably 50-300 bases; and

5) any complementary sequence segment should not occur with the base sequence of each primer or between the base sequences of the primers.

As used herein, the “adjacent regions” to a polymorphic site mean that an area containing both of a polymorphic site and adjacent regions thereto is within a distance suitable for nucleic acid amplification, preferably PCR. The adjacent regions amplified preferably have a length within the range of, but not limited to, about 50 bases to about 3000 bases, more preferably about 50 bases to about 2000 bases. To facilitate identification of polymorphisms, the product length is preferably 50-300 bases, more preferably about 100 bases. The adjacent regions preferably have a length within the range of, but not limited to, about 0 to about 3000 bases, more preferably about 0 to about 2000 bases, still more preferably about 0 to about 1000 bases on the 5′ or 3′ side of a polymorphic site.

Procedures and conditions for the nucleic acid amplification reaction are not specifically limited and are well known to those skilled in the art. Appropriate conditions can be applied by those skilled in the art depending on various factors such as the base sequence of the polymorphic site and adjacent regions thereto, the base sequence and length of the primer pair, etc. Generally, the nucleic acid amplification reaction can be performed under more stringent conditions (annealing reaction and nucleic acid elongation reaction at higher temperatures and less cycles) as the primer pair is longer or the proportion of G+C is higher or the distribution of A, T, G and C is evener. The use of more stringent conditions allows an amplification reaction with higher specificity.

The amplification reaction can be performed under conditions of, but not limited to, one cycle of 94° C. for 2 min, 30 cycles of 94° C. for 1 min, 58° C. for 1 min and 72° C. for 2 min, and finally one cycle of 72° C. for 2 min using 50 ng of a genomic DNA as a template, 200 μM of each dNTP and 5 U of ExTaq™ (TAKARA). The reaction can also be performed under conditions of one cycle of 94° C. for 2 min, 30 cycles of 94° C. for 1 min, 58° C. for 1 min and 72° C. for 1 min, and finally one cycle of 72° C. for 2 min. In another embodiment, the reaction can also be performed under conditions of one cycle of 94° C. for 2 min, 35 cycles of 94° C. for 30 sec, 58° C. for 30 sec and 72° C. for 30 sec, and finally one cycle of 72° C. for 2 min.

The subject rice (test rice) genomic DNA used as a template for PCR can be easily extracted from individuals or seeds by the method of Edwards et al. (Nucleic Acids Res. 8(6):1349, 1991). More preferably, DNA purified by standard techniques is used. An especially preferred extraction method is the CTAB method (Murray, M. G. et al., Nucleic Acids Res. 8(19):4321-5, 1980). The DNA is preferably used as a template for PCR at a final concentration of 0.5 ng/μL.

2) Preparation of Markers for Detecting Polymorphisms

After examining whether or not a polymorphism is detected in the amplification product by the nucleic acid amplification reaction with a pair of primers, a marker for detecting the polymorphism is prepared on the basis of the polymorphism found. Non-limiting examples of polymorphisms that can be detected in the amplification product are as follows.

a) A change containing a deleted region exists in the polymorphism in the nucleic acid amplification product.

In this case, a pair of primers for nucleic acid amplification are prepared to flank the deleted region to form a marker for detecting the polymorphism. If the deleted region has a sufficient size, the polymorphism can be detected from the difference in mobility by electrophoresing the amplification product on an agarose gel or an acrylamide gel, for example. The polymorphism can be detected when the difference in base pair numbers is about 5% or more in the case of agarose gel electrophoresis or when the difference in length is about 1 base or more in the case of sequencing acrylamide gel electrophoresis, for example. Alternatively, the polymorphism can be detected by hybridizing the nucleic acid amplification product using an oligobase or a DNA fragment having a complementary sequence to the base sequence excluding the deleted region as an analytical probe. Alternatively, the polymorphism can be confirmed by determining the base sequence of the amplification product, if desired. Known techniques for electrophoresis of nucleic acids, hybridization, sequencing and the like can be used as appropriate by those skilled in the art. In this case, the difference in the length of the amplification product directly reflects the polymorphism and markers for detecting polymorphisms on this basis are called ALP (amplicon length polymorphism) markers.

b) A base change causing a difference in restriction enzyme recognition exists in the polymorphism in the nucleic acid amplification product.

In this case, a pair of primers for nucleic acid amplification are prepared to flank the base change site to form a marker for detecting the polymorphism. In this case, a base change causing a difference in restriction enzyme recognition occurs in the polymorphism of the nucleic acid amplification product, i.e. the nucleic acid amplification product may be cleaved or not with one or more specific restriction enzymes. Thus, the amplification product can be treated with the restriction enzymes and electrophoresed on e.g. an agarose gel to detect the polymorphism from the difference in mobility. The polymorphism can be confirmed by determining the base sequence of the amplification product, if desired.

In this case, the difference in the length of the restriction fragment of the amplification product by PCR or the like reflects the polymorphism and markers for detecting polymorphisms on this basis are called CAPS markers or PCR-RFLP markers (A. Konieczny et al., supra.)

This is exemplified by primer pairs P4497 MboI, P23945 MboI, P41030 TaqI, P45177 BstUI, B59066 BsaJI and B56691 XbaI in Example 1 below. Even if the polymorphism can be detected by the length of the nucleic acid amplification product as described in a) above, the polymorphism can be more easily detected by combination with restriction enzyme treatment.

c) A base change causing no difference in restriction enzyme recognition exists in the polymorphism in the nucleic acid amplification product.

In this case, a pair of primers for introducing a mismatch are prepared that contains the base change site and alters a region containing the base change site into a base sequence causing a difference in restriction enzyme recognition in the nucleic acid amplification product to form a marker for detecting the polymorphism.

Specifically, a pair of primers based on the base sequences of regions naturally proximal to the Rf-1 gene cause a polymorphism in the nucleic acid amplification product but no difference in restriction enzyme recognition, and therefore, a mismatch is introduced into one or both of the primers to alter a region containing the base change site (polymorphism) into a base sequence causing a difference in restriction enzyme recognition in the nucleic acid amplification product. For example, the method described in Mikaelian et al., Nucl. Acids. Res. 20:376.1992 can be used as a standard technique for substituting, deleting or adding a specific base by PCR-mediated site-specific mutagenesis. The amplification product using the mismatch-introducing primers as a marker for detecting the polymorphism may be cleaved or not with one or more specific restriction enzymes because it has a difference in restriction enzyme recognition at the mismatch-introducing site. Therefore, the amplification product can be treated with the restriction enzymes and electrophoresed on e.g. an agarose gel to detect the polymorphism from the difference in mobility, as described in b) above.

The introduction of a mismatch must not affect not only the binding of the primers to a target plant genome but also the polymorphic base change. The polymorphic base change is used to introduce a mismatch near it so that a difference in restriction enzyme recognition occurs by a combination of both base change and mismatch. Methods for introducing such a mismatch are known to those skilled in the art and described in detail in Michaels, S. D. and Amasino, R. M. (1998), Neff, M. M., Neff, J. D., Chory, J. and Pepper, A. E. (1998), for example.

Markers in this case are improved CAPS markers described in b) above and called dCAPS (derived CAPS) markers. This is exemplified by P9493 BslI in Example 3 below.

If there are many extra restriction sites unrelated to varietal polymorphisms in the case of b) or c) above, it may be difficult to discern any difference in restriction site recognition based on polymorphisms. In this case, a mismatch may be introduced into a primer as appropriate to abolish unnecessary restriction sites. For example, a mismatch was introduced into the R-primer to abolish the MspI site unrelated to polymorphisms in B60304 MspI in Example 3.

Although the invention is not limited to any specific method, CAPS or dCAPS methods have several advantages over other RFLP methods. Specifically, analyses can be made with smaller amounts of samples than in RFLP, for example. Another advantage is that the time and labor required for analyses can be greatly reduced. Polymorphisms detected with PCR markers can be visualized by agarose gel electrophoresis that is easier than acrylamide gel electrophoresis used for microsatellite markers.

Preferred Embodiments of the Discerning Method of the Present Invention

Preferred embodiments of the method for discerning whether or not a subject rice has the Rf-1 gene are described below for illustrative purposes. In the examples herein, it was found that the base sequence of an indica variety IR24 carrying the Rf-1 gene (SEQ ID NO: 27) has at least the following polymorphisms 1)-8) as compared with corresponding regions of japonica varieties:

1) a base corresponding to the base 1239 of SEQ ID NO: 27 is A;

2) a base corresponding to the base 6227 of SEQ ID NO: 27 is A;

3) a base corresponding to the base 20680 of SEQ ID NO: 27 is G;

4) a base corresponding to the base 45461 of SEQ ID NO: 27 is A;

5) a base corresponding to the base 49609 of SEQ ID NO: 27 is A;

6) a base corresponding to the base 56368 of SEQ ID NO: 27 is T;

7) a base corresponding to the base 57629 of SEQ ID NO: 27 is C; and

8) a base corresponding to the base 66267 of SEQ ID NO: 27 is G.

In preferred embodiments of the present invention, therefore, the subject rice individual or seed is judged as carrying the Rf-1 gene when one to all of the requirements 1)-8) above are met.

We further verified that a region essential for the expression of the function of the Rf-1 gene is contained in especially bases 38538-54123, preferably bases 42357-53743, more preferably bases 42132-48883 in the base sequence of SEQ ID NO: 27. In an embodiment of the present invention, therefore, the subject rice individual or seed is determined to have the Rf-1 gene in the case that the nucleic acid having a base sequence which is identical to at least 70% of the base sequence of SEQ ID NO.27 or of the base sequence of bases 38538-54123 of SEQ ID NO.27, meets at least one of the following requirements 1) and 2):

1) a base corresponding to the base 45461 of SEQ ID NO.27 is A; and

2) a base corresponding to the base 49609 of SEQ ID NO.27 is A.

Known polymorphism detecting methods can be used to determine whether or not the above requirements are met. The base sequence of adjacent regions containing said sequence can also be directly determined. However, CAPS or dCAPS methods described above are preferably used because they are rapid and convenient. CAPS or dCAPS methods can be performed by a protocol comprising, for example:

i) preparing a pair of primers based on a base sequence of adjacent regions including any one of the following base;

1) a base corresponding to the base 1239 of SEQ ID NO: 27;

2) a base corresponding to the base 6227 of SEQ ID NO: 27;

3) a base corresponding to the base 20680 of SEQ ID NO: 27;

4) a base corresponding to the base 45461 of SEQ ID NO: 27;

5) a base corresponding to the base 49609 of SEQ ID NO: 27;

6) a base corresponding to the base 56368 of SEQ ID NO: 27;

7) a base corresponding to the base 57629 of SEQ ID NO: 27; and

8) a base corresponding to the base 66267 of SEQ ID NO: 27 is G.

to amplify both the base of the above and adjacent regions thereto;

ii) performing nucleic acid amplification reaction(s) using the genome DNA of the subject rice individual or the seed thereof as a template; and

iii) discerning the presence of the Rf-1 in the subject rice individual or the seed thereof based on polymorphism found in said nucleic acid amplification product.

The detection of polymorphisms in the nucleic acid amplification product is performed by, but not limited to, discerning the subject rice individual or seed to have the Rf-1 gene when one to all of the requirements 1)-8) below are met:

1) a region including a base corresponding to the base 1239 of SEQ ID NO: 27 does not have any MboI recognition sequence;

2) a region including a base corresponding to the base 6227 of SEQ ID NO: 27 does not have any BslI recognition sequence;

3) a region including a base corresponding to the base 20680 of SEQ ID NO: 27 does not have any MboI recognition sequence;

4) a region including a base corresponding to the base 45461 of SEQ ID NO: 27 does not have any TaqI recognition sequence;

5) a region including a base corresponding to the base 49609 of SEQ ID NO: 27 does not have any BstUI recognition sequence;

6) a region including a base corresponding to the base 56368 of SEQ ID NO: 27 does not have any MspI recognition sequence;

7) a region including a base corresponding to the base 57629 of SEQ ID NO: 27 does not have any BsaJI recognition sequence; and

8) a region including a base corresponding to the base 66267 of SEQ ID NO: 27 does not have any XbaI recognition sequence.

However, the present invention is not limited to the restriction enzymes above so far as each polymorphism in the specific regions 1)-8) above can be detected.

Preferably, identification methods of the present invention comprise:

i) preparing a pair of primers based on a base sequence of adjacent regions including any one of the following base;

1) a base corresponding to the base 45461; or

2) a base corresponding to the base 49609; to amplify both the base of the above and adjacent regions thereto;

ii) performing nucleic acid amplification reaction(s) using the genome DNA of the subject rice individual or the seed thereof as a template; and

iii) discerning the presence of the Rf-1 in the subject rice individual or the seed thereof based on polymorphism found in said nucleic acid amplification product. The subject rice individual or seed thereof is determined to have the Rf-1 gene in step iii), although not limited to, when at least one of the following requirements 1) and 2) is met:

1) a region including a base corresponding to the base 45461 of SEQ ID NO: 27 does not have any TaqI recognition sequence;

2) a region including a base corresponding to the base 49609 of SEQ ID NO: 27 does not have any BstUI recognition sequence.

Primer pairs used for the amplification reaction can be appropriately selected by those skilled in the art to preferably satisfy the conditions above on the basis of the base sequence of SEQ ID NO: 27. Preferably, any primer pair having a base sequence selected from the group consisting of SEQ ID NOS: 39 and 40, SEQ ID NOS: 41 and 42, SEQ ID NOS: 43 and 44, SEQ ID NOS: 45 and 46, SEQ ID NOS: 47 and 48, SEQ ID NOS: 49 and 50, SEQ ID NOS: 51 and 52, and SEQ ID NOS: 53 and 54 is used. More preferably, the primer pair is selected from the group consisting of SEQ ID NOS: 45 and 46, and SEQ ID NOS: 47 and 48. If necessary, the sequences of the above primer pairs containing substitutions, deletions or additions while retaining the binding specificity for the base sequence of the polymorphic site and adjacent regions thereto can also be used as primers.

To examine the resulting PCR product for restriction fragment length polymorphisms, it is cleaved with restriction enzymes corresponding to the restriction sites present in PCR markers. Such cleavage is accomplished by incubation for several hours to a day at the recommended reaction temperature for the restriction enzymes used. The PCR amplified sample cleaved with the restriction enzymes can be analyzed by electrophoresis on an about 0.7%-2% agarose gel or an about 3% MetaPhor™ agarose gel. The gel is visualized under UV light in ethidium bromide, for example.

In the most preferred embodiments of the present invention, restriction enzyme cleavage patterns show the bands as shown in Table 2 below on the visualized gel depending on the primer pair used.

TABLE 2

| | Approximate size |
|---|---|
| | (bp) of detected band |
| Amplified with P4497 MobI | |
| (SEQ ID NOS: 39 and 40) | |
| Restriction enzyme MboI | |
| Test rice genome having | 730 |
| the Rf-1 gene (homozygous): | |
| no: | 385, 345 |
| Amplified with P9493 BslI | |
| (SEQ ID NOS: 41 and 42) | |
| Restriction enzyme BslI | |
| Test rice genome having | 126 |
| the Rf-1 gene (homozygous): | |
| no: | 100, 26 |
| Amplified with P23945 MboI | |
| (SEQ ID NOS: 43 and 44) | |
| Restriction enzyme MboI | |
| Test rice genome having | 160, 100 |
| the Rf-1 gene (homozygous): | |
| no: | 260 |
| Amplified with P41030 TaqI | |
| (SEQ ID NOS: 45 and 46) | |
| Restriction enzyme TaqI | |
| Test rice genome having | 280 |
| the Rf-1 gene (homozygous): | |
| no: | 90, 190 |
| Amplified with P45177 | |
| BstUI (SEQ ID NOS: 47 and 48) | |
| Restriction enzyme BstUI | |
| Test rice genome having | 20, 65, 730 |
| the Rf-1 gene (homozygous): | |
| no: | 20, 65, 175, 555 |
| Amplified with B60304 | |
| MspI (SEQ ID NOS: 49 and 50) | |
| Restriction enzyme MspI | |
| Test rice genome having | 330 |
| the Rf-1 gene (homozygous): | |
| no: | 220, 110 |
| Amplified with B59066 BsaJI | |
| (SEQ ID NOS: 51 and 52) | |
| Restriction enzyme BsaJI | |
| Test rice genome having | 420 |
| the Rf-1 gene (homozygous): | |
| no: | 65, 355 |
| Amplified with B56691 XbaI | |
| (SEQ ID NOS: 53 and 54) | |
| Restriction enzyme XbaI | |
| Test rice genome having | 670 |
| the Rf-1 gene (homozygous): | |
| no: | 140, 530 |

In Example 3 below, recombinants proximal to the Rf-1 gene having pollen fertility (RS1-RS2, RC1-RC8) were tested for the chromosomal organization of the Rf-1 region using 14 polymorphic markers including the 8 primer pairs described above. As a result, it was confirmed that all the plants carry the Rf-1 gene derived from the indica variety between P9493 BslI and 59066 BsaJI. This result showed that recombinant pollens having the chromosomal organization as shown in FIG. 3 have pollen fertility, i.e. the Rf-1 gene is functional in these pollens. This means that a sequence determining the presence of the function of the Rf-1 gene is included in the indica region common to these recombinant pollens, i.e. in a region from the P4497 MboI to B56691 XbaI loci (about 65 kb) as estimated at maximum.

In the present invention, chromosomal walking was started on the presumption that the S12564 Tsp509I locus should be vary proximal to the Rf-1 locus as judged from the frequency of appearance of individuals by crossing. In fact, the genetic distance between both loci has been calculated to be about 0.04 cM as the result of the high-precision segregation analysis of the present invention. Even one of markers known to be most closely linked to the Rf-1 locus as described in Japanese Patent Public Disclosure No. 2000-139465 is reported to have a genetic distance of 1 cM from the Rf-1 locus. Considering that 1 cM is estimated to be equivalent to 300 kb on average in rice, a considerable time should be required to restrict the Rf-1 gene region if chromosomal walking were started from the marker described in Japanese Patent Public Disclosure No. 2000-139465.

VI. Method for Inhibiting the Function of Rf-1 Gene to Restore Fertility

According to the present invention, the nucleic acid containing the locus of a fertility restorer gene (Rf-1) including the nucleic acids which function to restore fertility was isolated. The entire base sequence thereof was determined, whereby the fertility restoring function of the Rf-1 gene can be controlled by genetic engineering techniques. Thus, the present invention further provides a method for inhibiting the function of Rf-1 to restore fertility.

A method for inhibiting the function of the Rf-1 gene to restore fertility according to one embodiment of the present invention comprises introducing an antisense having at least 100 continuous bases in length, and having a base sequence complementary to a nucleic acid having the base sequence of SEQ ID NO.27, or to a nucleic acid having a base sequence which is identical to at least 70% of the base sequence of SEQ ID NO.27, and which functions to restore fertility.

In an embodiment, the method for inhibiting the function of the Rf-1 gene to restore fertility according to the present invention comprises introducing an antisense having at least 100 continuous bases in length, and being selected from base sequences complementary to a nucleic acid having the base sequence of bases 38538-54123, preferably bases 42357-53743, more preferably bases 42132-48883 of SEQ ID NO: 27, or to a nucleic acid having a base sequence which is identical to at least 70% of the base sequence of bases 38538-54123, preferably bases 42357-53743, more preferably bases 42132-48883 of SEQ ID NO: 27 and, which functions to restore fertility.

The antisense has a length of at least 100 bases or more, more preferably 500 bases or more, most preferably 1000 bases or more. From the viewpoint of technical convenience of introduction, it preferably has a length of 10000 bases or less, more preferably 5000 bases or less. The antisense can be synthesized by known methods. The antisense can be introduced into rice by known methods as described in e.g. Terada et al. (Plant Cell Physiol. 2000 July, 41(7), pp. 881-888).

It is also anticipated that Rf-1 disrupted lines can be established by screening variant lines containing a transposable element such as, but not limited to, Tos17 (Hirochika H. et al. 1996, Proc. Natl. Acad. Sci. USA 93, pp. 7783-7788) for a line containing the transposable element in the base sequence of SEQ ID NO: 27. In plants, gene disruption by homologous recombination has been studied. It may also be possible to inhibit fertility restoring function by establishing such a line in which the Rf-1 gene has been replaced by a variant Rf-1 gene using a nucleic acid having the base sequence of SEQ ID NO.27, or a nucleic acid having a base sequence which is identical to at least 70% of the base sequence of SEQ ID NO.27.

### REFERENCES

1. Fukuta et al. 1992, Jpn J. Breed. 42 (supl. 1) p. 164-165.
2. Japanese Patent Public Disclosure No. HEI7(1995)-222588.
3. Japanese Patent Public Disclosure No. HEI9(1997)-313187.
4. Japanese Patent Public Disclosure No. 2000-139465.
5. Harushima et al. 1998, Genetics 148 p. 479-494.
6. Michaels and Amasino 1998, The Plant Journal 14(3) p. 381-385.
7. Neff et al. 1998, The plant Journal 14(3) p. 387-392.
8. D. E. Harry, et al., Theor Appl Genet (1998) 97:p. 327-336.
9. Hiei et al., Plant Journal (1994), 6(2), p. 272-282.
10. Komari et al., Plant Journal (1996) 10, p. 165-174.
11. Ditta et al., Proc. Natl. Acad. Sci. USA (1980), 77: p. 7347-7351,
12. P. Vos et al., Nucleic Acids Res. Vol. 23, p. 4407-4414 (1995).
13. O. Parnaud, X. et al, Mol. Gen. Genet. (1996) 252:p. 597-607.
14. A. Konieczny et al., (1993), Plant J. 4(2) p. 403-410.
15. Edwards et al., Nucleic Acids Res. 8(6): 1349, 1991.
16. Murray M. G. et al., Nucleic Acids Res. 8(19):4321-5, 1980.
17. Terada et al., Plant Cell Physiol. 2000 July, 41(7), p. 881-888.
18. Hirochika H. et al. 1996, Proc. Natl. Acad. Sci. USA 93, p. 7783-7788.

### EXAMPLES

The following examples further illustrate the present invention but are not intended to limit the technical scope of the invention. Those skilled in the art can readily add modifications/changes to the present invention on the basis of the description of the specification, and those modifications/changes are included in the technical scope of the present invention.

### REFERENCE EXAMPLES

The following reference examples are based on the examples described in our prior application (Japanese Patent Application No. 2000-247204 filed Aug. 17, 2000).

### Reference Example 1

### Conversion of RFLP Markers Around Rf-1 Gene to PCR Markers

In this reference example, nine RFLP markers (i.e., R1877, G291, R2303, S12564, C1361, S10019, G4003, S10602 and G2155) around the locus of Rf-1 gene were converted to PCR markers.

(1) Materials and Methods

The following nine RFLP markers, R1877, G291, R2303, S12564, C1361, S10019, G4003, S10602 and G2155, were purchased from the National Institute of Agrobiological Sciences, the Ministry of Agriculture, Forestry and Fisheries of Japan. After determining the base sequences of the inserts in the vectors, experiments were conducted according to the following procedures. Among rice varieties herein, Asominori belongs to japonica, and IR24 belongs to indica.

(2) Preparation of Asominori Genomic Library

Total DNA was extracted from green leaves of Asominori by the CTAB method. After partial digestion with MboI, the DNA was fractionated according to size by NaCl density gradient centrifugation (6-20% linear gradient, 20° C., 37,000 rpm, 4 hr, total volume=12 mL). A portion of each fraction (about 0.5 mL) was subjected to electrophoresis and fractions containing 15-20 kb DNA were collected and purified. A library was constructed using Lambda DASH II (Stratagene) as a vector in accordance with the attached protocol. Giga Pack III Gold (Stratagene) was used for packaging. After packaging, 500 μL of SM Buffer and 20 μL of chloroform were added. After centrifugation, 20 μL of chloroform was added to the supernatant to make a library solution.

XL-1 Blue MRA (P2) was infected with 5 μL of a 50-fold dilution of the library solution, whereupon 83 plaques were formed. This corresponded to 4.15×10⁵ pfu per library, and hence, it was calculated that the plaques covered 8.3×10⁹ bp assuming that the average length of the inserted fragments was 20 kb. The library was therefore considered to have an adequate size for the rice genome (4×10⁸ bp).

(3) Isolation of Genomic Clones Containing R1877-, C1361- and G4003-Marker Regions.

As for C1361 and G4003, plasmids containing the RFLP marker probe were isolated and subjected to restriction enzyme treatment and electrophoresis to separate the RFLP marker probe portion; the desired DNA was recovered on a DNA recovery filter (Takara SUPREC-01). As for R1877, primers were designed that were specific to both ends of the marker probe and PCR was performed with the total DNA of Asominori used as a template; the amplification products were electrophoresed and recovered by the method described above. The recovered DNA was labelled with a Rediprime DNA Labelling System (Amersham Pharmacia) to prepare a probe for screening the library. PCR was performed in the usual manner (this also applies to the following description).

Screening of the library was performed in the usual manner after blotting the plaques onto Hybond-N+ (Amersham Pharmacia). After primary screening, areas of positive plaques were individually punched out, suspended in SM buffer and subjected to the second round of screening. After the second screening, the positive plaques were punched out and subjected to the third round of screening to isolate a single plaque.

The isolated plaque of interest was suspended in SM buffer and primary multiplication of the phage was performed by the plate lysate method. The resulting phage-enriched solution was subjected to secondary multiplication by shake culture and the phage DNA was purified with Lambda starter kit (QIAGEN).

For each marker, primary screening was conducted on eight plates. A 10 μL aliquot of the library solution was employed per plate. After the primary, second and third rounds of screening, four genomic clones in association with R1877 were isolated and three were isolated in association with each of C1361 and G4003.

(4) Conversion of R1877 to PCR Marker

The isolated genomic clones were analyzed to identify the causative site of RFLP, or the EcoRI site that exists in IR24 (indica rice) but not in Asominori (japonica rice), thereby converting R1877 to a PCR marker.

Specifically, the four isolated clones were subjected to the following analyses. First, T3 and T7 primers were used to determine the base sequences at both ends of the insert in each clone. Then, primers extending outwardly from both ends of the marker probe were designed. They were combined with T3 and T7 primers to give a combination of four primers in total, and employed in PCR with each clone used as the template.

In a separate step, each clone was digested with NotI and EcoRI, and electrophoresed to estimate the insert size and the length of each EcoRI fragment.

These analyses revealed the relative positions of the individual clones. In RFLP analysis, marker probe R1877 was reported to detect an EcoRI fragment of 20 kb in Nipponbare (japonica rice) and one of 6.4 kb in Kasalath (indica rice)

(ftp://ftp.staff.or.jp/pub/geneticmap98/parentsouthern/chr10/R1877.JPG). This fact, taken together with the results of analysis described above, gave a putative position for the EcoRI site that existed in IR24 but not in Asominori. Hence, a primer combination (SEQ ID NO:1×SEQ ID NO:2) that was designed to amplify the nearby region was employed to perform genomic PCR over 30 cycles, each cycle consisting of 94° C.×1 min, 58° C.×1 min and 72° C.×2 min. The PCR product was treated with EcoRI and subjected to electrophoresis on 0.7% agarose gel.

As a result, the expected polymorphisms were observed between Asominori and IR24. By treatment with EcoRI, the PCR product (˜3200 bp) was cleaved to yield 1500 bp and 1700 bp fragments in IR24 but not in Asominori. Mapping of the marker was made with an RIL (recombinant inbred line) of Asominori-IR24 with the results that the PCR marker was located in the same region as that of RFLP marker locus R1877, thereby confirming the conversion of RFLP marker R1877 to a PCR marker, which was named R1877 EcoRI in the present invention.

(5) Conversion of G4003 to PCR Marker

The isolated genomic clones were analyzed to identify the causative site of RFLP, or the HindIII site that existed in Asominori but not in IR24, thereby converting G4003 to a PCR marker.

By performing analyses similar to those employed for R1877, the relative positions of the three isolated clones were revealed. In RFLP analysis, marker probe G4003 was reported to detect a HindIII fragment of 3 kb in Nipponbare (japonica rice) and one of 10 kb in Kasalath (indica rice) (ftp://ftp.staff.or.jp/pub/geneticmap98/parentsouthern/chr10/R1877.JPG). This report, taken together with the analyses described above, led to a temporary conclusion that the HindIII site that existed in Asominori but not in IR24 would be at either one of two candidate sites. Hence, a primer combination (SEQ ID NOS: 3 and 4) that was designed to amplify the area in the neighborhood of each HindIII site was employed to perform genomic PCR over 35 cycles, each cycle consisting of 94° C.×30 sec, 58° C.×30 sec and 72° C.×30 sec. The PCR product was treated with HindIII and subjected to electrophoresis on 2% agarose gel. As a result, the HindIII site within the marker probe was found to have polymorphisms. By treatment with HindIII, the PCR product (362 bp) was cleaved to yield a 95 bp fragment and a 267 bp fragment in Asominori but not in IR24. Mapping of the site demonstrated the conversion of RFLP marker G4003 to a PCR marker, which was named G4003 HindIII (SEQ ID NO:19) in the present invention.

(6) Conversion of C1361 to PCR Marker

Primers were designed on the basis of the base sequence information of the isolated genomic clones. PCR was performed with the total DNAs of Asominori and IR24 being used as a template and the PCR product was recovered by known methods after electrophoresis. Using the recovered DNA as a template, the inventors analyzed the base sequence of each of the rice varieties with ABI Model 310 in search of mutations that would cause polymorphisms.

By performing analyses similar to those employed for R1877, approximate relative positions of the three isolated clones could be established. As it turned out, however, regions around the C1361 marker would be difficult to amplify by PCR or determine their base sequences, and hence, it would not be easy to identify the causative site of RFLP. Hence, the inventors took notice of the region capable of yielding a comparatively long PCR product (2.7 kb) and made an attempt to create a dCAPS marker.

Specifically, upon comparing the base sequences of the genomic PCR products of said region using Asominori and Koshihikari (both japonica rice) and Kasalath and IR24 (both indica rice), the inventors found six sites of polymorphism between japonica and indica. One of these six sites was used to create a dCAPS marker. To this end, with SEQ ID NO:5 and SEQ ID NO:6 used as primers, PCR was performed over 35 cycles, each cycle consisting of 94° C.×30 sec, 58° C.×30 sec and 72° C.×30 sec. The PCR product was treated with MwoI and analyzed by electrophoresis on 3% MetaPhor™ agarose gel. In Asominori, cleavage occurred at two sites to give three observable bands of about 25 bp, 50 bp and 79 bp, but in IR24 cleavage occurred at one site to give two observable bands of about 50 bp and 107 bp. Mapping demonstrated the conversion of RFLP marker C1361 to a PCR marker, which was named C1361 MwoI (SEQ ID NO:20) in the present invention.

(7) Conversion of G2155 to PCR Marker

Primers specific to both ends of the marker probe were designed and PCR was performed with the total DNA of Asominori, Koshihikari, IR24 or IL216 (a line produced by introducing Rf-1 gene into Koshihikari by back crossing; its genotype was Rf-1/Rf-1) being used as a template. Purification of the PCR product and searching for a mutation that would be useful for providing restriction fragment polymorphisms were performed by the methods already described above.

Specifically, as a result of comparing the base sequences of corresponding regions of the varieties under test, mutations were found at three sites between the variety/line (IR24 and IL216) having Rf-1 gene and the variety (Asominori and Koshihikari) not having Rf-1 gene. One of the three sites was utilized to create a dCAPS marker. To this end, SEQ ID NO:7 and SEQ ID NO:8 were used as primers to perform PCR over 35 cycles, each cycle consisting of 94° C.×30 sec, 58° C.×30 sec and 72° C.×30 sec. The PCR product was treated with MwoI and analyzed by electrophoresis on 3% MetaPhor™ agarose gel. In Asominori, cleavage occurred at one site to give two observable bands of about 25 bp and 105 bp, but in IR24, cleavage occurred at two sites to give three observable bands of about 25 bp, 27 bp and 78 bp. Mapping demonstrated the conversion of RFLP marker G2155 to a PCR marker, which was named G2155 MwoI (SEQ ID NO:21) in the present invention.

(8) Conversion of G291 to PCR Marker

Primers specific to internal sequences of the marker probe were designed and used in various combinations to perform PCR to find a primer combination that could yield an amplification product of the expected size. Using the selected primer combination, the inventors performed PCR with the total DNA of Asominori, Koshihikari, IR24 and IL216 used as a template. Purification of the PCR product and searching for a mutation that could be utilized in providing restriction fragment polymorphisms were performed by the methods already described above.

Specifically, using the primers designed to be specific for the marker probe sequence, the inventors performed genomic PCR of each variety under test and compared the base sequences of the products. As a result, mutations were found at four sites between the variety/line having Rf-1 gene (IR24 and IL216) and the variety (Asominori and Koshihikari) not having Rf-1 gene. One of the four sites was used to create a dCAPS marker. To this end, SEQ ID NO:9 and SEQ ID NO:10 were used as primers to perform PCR over 35 cycles, each cycle consisting of 94° C.×30 sec, 58° C.×30 sec and 72° C.×30 sec. The PCR product was treated with MspI and analyzed by electrophoresis on 3% MetaPhor™ agarose gel. In the varieties/lines having Rf-1 gene, cleavage occurred at two sites to give three observable bands of about 25 bp, 49 bp and 55 bp, but in the varieties not having Rf-1 gene, cleavage occurred at one site to give two observable bands of about 25 bp and 104 bp. Mapping demonstrated the conversion of RFLP marker G291 to a PCR marker, which was named G291 MspI (SEQ ID NO:22) in the present invention.

(9) Conversion of R2303 to PCR Marker

Primers specific to internal sequences of the marker probe were designed and PCR was performed with the total DNA of Asominori (japonica rice) and IR24 and Kasalath (indica rice) used as a template. Purification of the PCR product and searching for a mutation that could be used for providing restriction fragment polymorphisms were performed by the methods already described above.

As a result of comparing the base sequences of corresponding regions of the varieties under test, a mutation was found between japonica rice and indica rice. Since the mutation occurred at the BslI recognition site, the site was directly used to create a CAPS marker. To this end, SEQ ID NO:11 and SEQ ID NO:12 were used as primers and PCR was performed over 30 cycles, each cycle consisting of 94° C.×1 min, 58° C.×1 min and 72° C.×2 min. The PCR product was treated with BslI and analyzed by electrophoresis on 2% agarose gel. In japonica rice, cleavage occurred at one site to give two observable bands of about 238 bp and 1334 bp, but in indica rice, cleavage occurred at two sites to give three observable bands of about 238 bp, 655 bp and 679 bp. Mapping demonstrated the conversion of RFLP marker R2303 to a PCR marker, which was named R2303 BslI (SEQ ID NO:23) in the present invention.

(10) Converting S10019 to PCR Marker

S10019 was converted to a PCR marker in accordance with the method (9) of converting R2303 to a PCR marker.

Specifically, as a result of comparing the base sequences of corresponding regions of the varieties under test, a mutation was found between japonica rice and indica rice. Since the mutation occurred at the BstUI recognition site, the site was directly used to create a CAPS marker. To this end, SEQ ID NO:13 and SEQ ID NO:14 were used as primers and PCR was performed over 30 cycles, each cycle consisting of 94° C.×1 min, 58° C.×1 min and 72° C.×1 min. The PCR product was treated with BstUI and analyzed by electrophoresis on 2% agarose gel. In japonica rice, cleavage occurred at one site to give two observable bands of about 130 bp and 462 bp, but in indica rice, cleavage occurred at two sites to give three observable bands of about 130 bp, 218 bp and 244 bp. Mapping demonstrated the conversion of RFLP marker S10019 to a PCR marker, which was named S10019 BstUI (SEQ ID NO:24) in the present invention.

(11) Conversion of S10602 to PCR Marker

S10602 was converted to a PCR marker in accordance with the method (9) of converting R2303 to a PCR marker.

Specifically, as a result of comparing the base sequences of corresponding regions of the varieties under test, a mutation was found between japonica rice and indica rice. The mutation was used to create a CAPS marker. To this end, SEQ ID NO:15 and SEQ ID NO:16 were used as primers and PCR was performed over 33 cycles, each cycle consisting of 94° C.×1 min, 58° C.×1 min and 72° C.×1 min. The PCR product was treated with KpnI and analyzed by electrophoresis on 2% agarose gel. In japonica rice, cleavage occurred at one site to give two observable bands of about 117 bp and 607 bp, but in indica rice, no cleavage occurred, giving only an observable band of 724 bp. Mapping demonstrated the conversion of RFLP marker S10602 to a PCR marker, which was named S10602 KpnI (SEQ ID NO:25) in the present invention.

(12) Conversion of S12564 to PCR Marker

S12564 was converted to a PCR marker in accordance with the method of converting R2303 to a PCR marker.

Specifically, as a result of comparing the base sequences of corresponding regions of the varieties under test, a mutation was found between japonica rice and indica rice. The mutation was used to create a dCAPS marker. To this end, SEQ ID NO:17 and SEQ ID NO:18 were used as primers and PCR was performed over 35 cycles, each cycle consisting of 94° C.×30 sec, 58° C.×30 sec and 72° C.×30 sec. The PCR product was treated with Tsp509I and analyzed by electrophoresis on 3% MetaPhor™ agarose gel. In japonica rice, cleavage occurred at two sites to give three observable bands of 26 bp, 41 bp and 91 bp, but in indica rice, cleavage occurred at one site to give two observable bands of 41 bp and 117 bp. Mapping demonstrated the conversion of RFLP marker S12564 to a PCR marker, which was named S12564 Tsp509I (SEQ ID NO:26) in the present invention.

### Reference Example 2

### Mapping of Rf-1 Gene Locus

DNA was extracted from 1042 seedlings of the F1 population produced by pollinating MS Koshihikari with MS-FR Koshihikari, and the DNA extract was used in the analysis. MS Koshihikari (generation: BC10F1) was created by replacing the cytoplasm of Koshihikari with BT type male sterility cytoplasm. MS-FR Koshihikari was a line created by introducing Rf-1 gene from IR8 (supplied from National Institute of Agrobiological Sciences) into MS Koshihikari (the locus of Rf-1 gene being heterozygous).

First, each individual was investigated for the genotype at two marker loci R1877 EcoRI and G2155 MwoI described in Reference example 1 that would presumably be located on opposite sides of the locus of Rf-1 gene. Japonica type homozygotes with respect to either locus R1877 EcRI or G2155 MwoI were regarded as recombinants between these two marker loci. Then, each of such recombinants was investigated for the genotypes of G291 MspI, R2303 BslI, S12564 Tsp 509I, C1361 MwoI, S10019 BstUI, G4003 HindIII and S10602 KpnI loci, and the positions of recombination were identified.

The genotype investigation with respect to R1877 EcoRI and G2155 MwoI loci revealed that 46 individuals were recombinants around the locus of Rf-1 gene. Genotypes of the marker loci around the locus of Rf-1 gene were investigated and the results are shown in Table 3.

TABLE 3
Genotypes of Marker Loci in Recombinant Individuals Around Rf-1 Locus

| | Locus | | | | | | | | | | | | | | | | | | | | | | |
|---|---|---|---|---|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 |
| R1877 EcoRI | J | J | J | J | J | J | J | J | H | H | H | H | H | H | H | H | H | H | H | H | H | H | H |
| G291 MspI | H | J | J | J | J | J | J | J | H | H | H | H | H | H | H | H | H | H | H | H | H | H | H |
| R2303 BsiI | H | H | J | J | J | J | J | J | H | H | H | H | H | H | H | H | H | H | H | H | H | H | H |
| S12564 Tsp509I | H | H | H | H | H | H | H | J | H | H | H | H | H | H | H | H | H | H | H | H | H | H | H |
| C1361 MwoI | H | H | H | H | H | H | H | H | J | J | H | H | H | H | H | H | H | H | H | H | H | H | H |
| S10019 BstUI | H | H | H | H | H | H | H | H | J | J | J | J | J | J | J | J | H | H | H | H | H | H | H |
| G4003 HindIII | H | H | H | H | H | H | H | H | J | J | J | J | J | J | J | J | J | J | J | J | J | J | J |
| S10602 KpnI | H | H | H | H | H | H | H | H | J | J | J | J | J | J | J | J | J | J | J | J | J | J | J |
| G2155 MwoI | H | H | H | H | H | H | H | H | J | J | J | J | J | J | J | J | J | J | J | J | J | J | J |

| 24 | 25 | 26 | 27 | 28 | 29 | 30 | 31 | 32 | 33 | 34 | 35 | 36 | 37 | 38 | 39 | 40 | 41 | 42 | 43 | 44 | 45 | 46 |
|---|---|---|---|---|---|---|---|---|---|---|---|---|---|---|---|---|---|---|---|---|---|---|
| H | H | H | H | H | H | H | H | H | H | H | H | H | H | H | H | H | H | H | H | H | H | H |
| H | H | H | H | H | H | H | H | H | H | H | H | H | H | H | H | H | H | H | H | H | H | H |
| H | H | H | H | H | H | H | H | H | H | H | H | H | H | H | M | H | H | H | H | H | H | H |
| H | H | H | H | H | H | H | H | H | H | H | H | H | H | H | H | H | H | H | H | H | H | H |
| H | H | H | H | H | H | H | H | H | H | H | H | H | H | H | H | H | H | H | H | H | H | H |
| H | H | H | H | H | H | H | H | H | H | H | H | H | H | H | H | H | H | H | H | H | H | H |
| J | J | J | J | J | J | J | J | J | H | H | H | H | H | H | H | H | H | H | H | H | M | H |
| J | J | J | J | J | J | J | J | J | J | J | J | J | J | J | J | J | J | J | J | J | H | M |
| J | J | J | J | J | J | J | J | J | J | J | J | J | J | J | J | J | J | J | J | J | J | J |

J: Homozygous Koshihikari type
H: Heterozygous Koshihikari type/MS-FR Koshihikari type

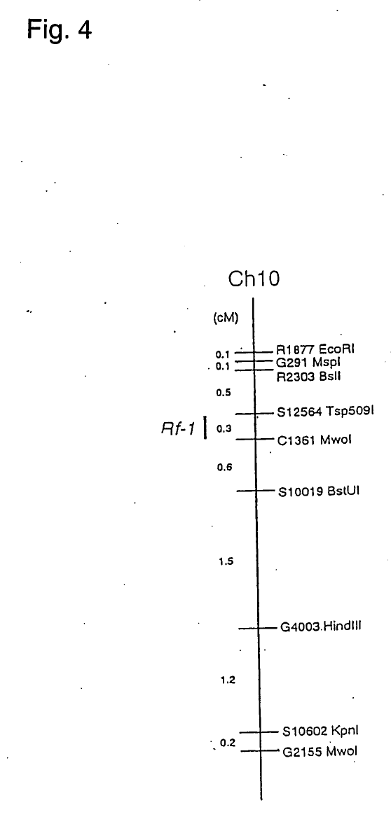

As shown in Table 3, recombinant 8 homozygous for japonica at the S12564 Tsp509I marker locus and recombinants 9 and 10 homozygous for japonica at the C1361 Mwo marker locus were obtained. As all of these recombinants restored fertility, the former was regarded as a recombinant between the Rf-1 and S12564 Tsp509I loci while the latter were regarded as recombinants between the Rf-1 and C1361 MwoI loci, showing that the Rf-1 gene is located between the S12564 Tsp509I and C1361 MwoI loci. Based on the report that only pollens carrying the Rf-1 gene have fertility in individuals having the BT type male sterile cytoplasm in the cross above (C. Shinjyo, JAPAN. J. GENETICS Vol. 44, No. 3:149-156 (1969)), the Rf-1 gene locus could be located on a detailed linkage map (FIG. 4).

### Example 1

### Acquisition of Recombinant Individuals Proximal to the Rf-1 Locus

(Materials and Methods)

DNA was extracted from each of 4103 individuals of BC10F1 population produced by pollinating MS Koshihikari (generation: BC10F1) with MS-FR Koshihikari (generation: BC9F1, heterozygous at the Rf-1 locus), and genotyped at the S12564 Tsp509I and C1361 MwoI loci in the same manner as described in Reference example 2 above. Individuals having a genotype homozygous for Koshihikari at the S12564 Tsp509I locus were regarded as those generated by recombination between the Rf-1 and S12564 Tsp509I loci, while individuals having a genotype homozygous for Koshihikari at the C1361 MwoI locus were regarded as those generated by recombination between the Rf-1 and C1361 MwoI loci.

(Results and Discussion)

A survey of 4103 individuals revealed one recombinant individual between the Rf-1 and S12564 Tsp509I loci and 6 recombinant individuals between the Rf-1 and C1361 MwoI loci. The previous survey of 1042 individuals obtained by crossing in Reference example 2 above had already revealed one recombinant individual between the Rf-1 and S12564 Tsp509I loci and 2 recombinant individuals between the Rf-1 and C1361 MwoI loci as shown in Table 3.

Thus, a total of 2 recombinant individuals between the Rf-1 and S12564 Tsp509I loci and 8 recombinant individuals between the Rf-1 and C1361 MwoI loci were able to be oabtained from 5145 individuals. These 10 individuals were tested by high-precision segregation analysis in the examples below.

### Example 2

### Chromosomal Walking

(1) First Chromosomal Walking

(Materials and Methods)

A genomic library was constructed from the genomic DNA of Asominori japonica (not carrying Rf-1) using Lambda DASH II vector as described in Reference example 1 and tested by chromosomal walking.

PCR was routinely performed using total DNA of Asominori as a template in combination with the following primer pair:

| | |
|---|---|
| 5′-atcaggagccttcaaattgggaac-3′ | (SEQ ID NO:29) |
| and | |
| 5′-ctcgcaaattgcttaattttgacc-3′ | (SEQ ID NO:30) |

designed for a partial base sequence (Accession No. D47284) of RFLP probe S12564. The resulting amplification products of about 1200 bp were electrophoresed on an agarose gel and then purified by QIAEXII (QIAGEN). The purified DNA was labeled with a rediprime DNA labelling system (Amersham Pharmacia) to give a library screening probe (probe A, FIG. 1).

The library was routinely screened after plaques were blotted onto Hybond-N⁺ (Amersham Pharmacia). Single plaques were separated, after which phage DNA was purified by the plate lysate method using Lambda Midi kit (QIAGEN).

(Results and Discussion)

The results of terminal base sequence analysis and restriction enzyme fragment length analysis showed that two (WSA1 and WSA3) of 4 clones obtained by screening were in a relative position as shown in FIG. 1. The Asominori genomic base sequences corresponding to WSA1 and WSA3 were determined by primer walking (DNA Sequencer 377, ABI).

(2) Second Chromosomal Walking

(Materials and Methods)

In addition to the Asominori genomic library described above, an IR24 genomic library was similarly constructed from the genomic DNA of an indica variety IR24 (carrying Rf-1) and tested by chromosomal walking.

PCR was routinely performed using DNA of WSA3 as a template in combination with the following primer pair:

| | |
|---|---|
| 5′-tgaaggagttatgggtgcgtgacg-3′ | (SEQ ID NO:31) |
| and | |
| 5′-ttgccgagcacacttgccatgtgc-3′ | (SEQ ID NO:32) |

designed for the Asominori genomic base sequence determined in (1). The resulting amplification products of 524 bp were purified and labeled by the method described above to give a library screening probe (probe E, FIG. 1).

Library screening and phage DNA purification were performed by the method described above.

(Results and Discussion)

The results of terminal base sequence analysis and restriction enzyme fragment length analysis showed that one (WSE8) of 15 clones obtained by screening of the Asominori genomic library was in a relative position as shown in FIG. 1. The Asominori genomic base sequence corresponding to WSE8 was determined by primer walking.

The results of terminal base sequence analysis and restriction enzyme fragment length analysis showed that two (XSE1 and XSE7) of 7 clones obtained by screening of the IR24 genomic library were in a relative position as shown in FIG. 1. The IR24 genomic base sequences corresponding to XSE1 and XSE7 were determined by primer walking.

(3) Third Chromosomal Walking

(Materials and Methods)

The Asominori genomic library and IR24 genomic library described above were tested by chromosomal walking.

PCR was routinely performed using DNA of WSE8 as a template in combination with the following primer pair:

| | |
|---|---|
| 5′-gcgacgcaatggacatagtgctcc-3′ | (SEQ ID NO:33) |
| and | |
| 5′-ttacctgccaagcaatatccatcg-3′ | (SEQ ID NO:34) |

designed for the Asominori genomic base sequence determined in (2). The resulting amplification products of 1159 bp were purified and labeled by the method described above to give a library screening probe (probe F, FIG. 1).

Library screening and phage DNA purification were performed by the method described above.

(Results and Discussion)

The results of terminal base sequence analysis and restriction enzyme fragment length analysis showed that two (WSF5 and WSF7) of 8 clones obtained by screening of the Asominori genomic library were in a relative position as shown in FIG. 1. The Asominori genomic base sequences corresponding to WSF5 and WSF7 were determined by primer walking.

The results of terminal base sequence analysis and restriction enzyme fragment length analysis showed that two (XSF4 and XSF20) of 13 clones obtained by screening of the IR24 genomic library were in a relative position as shown in FIG. 1. The IR24 genomic base sequences corresponding to XSF4 and XSF20 were determined by primer walking.

(4) Fourth Chromosomal Walking

(Materials and Methods)

The Asominori genomic library and IR24 genomic library described above were tested by chromosomal walking.

PCR was routinely performed using DNA of WSF7 as a template in combination with the following primer pair:

| | |
|---|---|
| 5′-aaggcatactcagtggagggcaag-3′ | (SEQ ID NO:35) |
| and | |
| 5′-ttaacctgaccgcaagcacctgtc-3′ | (SEQ ID NO:36) |

designed for the Asominori genomic base sequence determined in (3). The resulting amplification products of 456 bp were purified and labeled by the method described above to give a library screening probe (probe G, FIG. 1).

Library screening and phage DNA purification were performed by the method described above.

(Results and Discussion)

The results of terminal base sequence analysis and restriction enzyme fragment length analysis showed that two (WSG2 and WSG6) of 6 clones obtained by screening of the Asominori genomic library were in a relative position as shown in FIG. 1. The Asominori genomic base sequences corresponding to WSG2 and WSG6 were determined by primer walking.

The results of terminal base sequence analysis and restriction enzyme fragment length analysis showed that three (XSG8, XSG16 and XSG22) of 14 clones obtained by screening of the IR24 genomic library were in a relative position as shown in FIG. 1. The IR24 genomic base sequences corresponding to XSG8, XSG16 and XSG22 were determined by primer walking.

(5) Fifth Chromosomal Walking

(Materials and Methods)

The IR24 genomic library described above was tested by chromosomal walking.

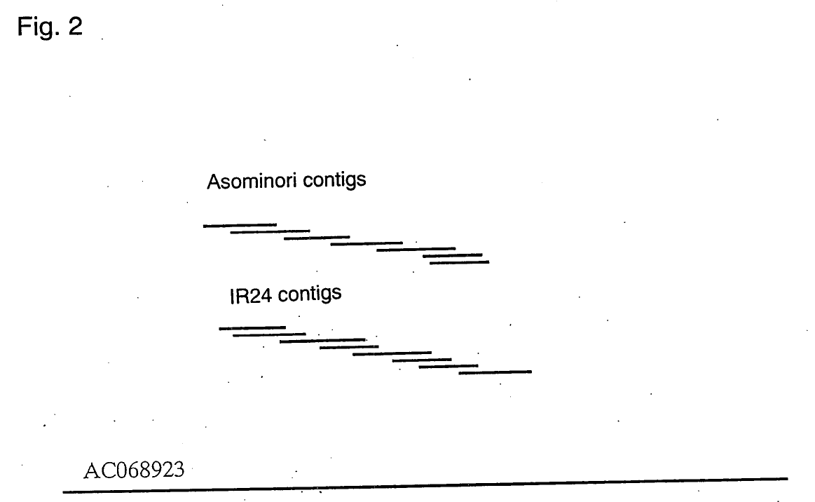

We perused the public website of TIGR (The Institute for Genomic Research) and found that a BAC (Bacterial Artificial Chromosome) clone (Accession No. AC068923) containing RFLP marker S12564 had been deposited with a public database (GenBank). This BAC clone contains the genomic DNA of Nipponbare japonica and it was shown from base sequence comparison to completely include the contig regions of Asominori and IR24 prepared in (1)-(4) (FIG. 2).

Thus, PCR was routinely performed using total DNA of IR24 as a template in combination with the following primer pair:

| | |
|---|---|
| 5′-tggatggactatgtggggtcagtc-3′ | (SEQ ID NO:37) |
| and | |
| 5′-agtggaagtggagagagtagggag-3′ | (SEQ ID NO:38) |

designed to amplify a part of this BAC clone. The resulting amplification products of about 600 bp were purified and labeled by the method described above to give a library screening probe (probe H, FIG. 1).

Library screening and phage DNA purification were performed by the method described above.

(Results and Discussion)

The results of terminal base sequence analysis and restriction enzyme fragment length analysis showed that one (XSH18) of 15 clones obtained by screening of the IR24 genomic library was in a relative position as shown in FIG. 1. The IR24 genomic base sequence corresponding to XSH18 was determined by primer walking.

### Example 3

### High Precision Segregation Analysis

(1) Development of PCR Marker P4497 MboI

Comparison between the genomic base sequence corresponding to the IR24 contig (SEQ ID NO: 27) and the genomic base sequence corresponding to the Asominori contig (SEQ ID NO: 28) determined in Example 2 revealed that the 1239th base of SEQ ID NO: 27 is A while the 12631st base of SEQ ID NO: 28 corresponding to said position is G.

For detecting this change, fragments of about 730 bp are first amplified by PCR from a region surrounding said position using the following primer pair:

| | |
|---|---|
| P4497 MboI F: | |
| 5′-ccctccaacacataaatggttgag-3′ | (SEQ ID NO:39) |

(corresponding to bases 853-876 of SEQ ID NO:27)
(corresponding to bases 12247-12270 of SEQ ID
NO:28)
and
P4497.MboI R:

| | |
|---|---|
| 5′-tttctgccaggaaactgttagatg-3′ | (SEQ ID NO:40) |

(corresponding to bases 1583-1560 of SEQ ID NO:27)
(corresponding to bases 12975-12952 of SEQ ID
NO:28).

The amplification products can be visualized by electrophoresis on an agarose gel after treatment with MboI. Thus, the change can be detected as a difference in mobility in the agarose gel due to the difference in the length of DNA after MboI treatment because the amplification products from Asominori DNA having an MboI recognition sequence (GATC) are cleaved with MboI while the amplification products from IR24 DNA are not cleaved with MboI for the lack of the MboI recognition sequence.
(2) Development of PCR Marker P9493 BslI

Comparison between the genomic base sequence corresponding to the IR24 contig (SEQ ID NO: 27) and the genomic base sequence corresponding to the Asominori contig (SEQ ID NO: 28) determined in Example 2 revealed that the 6227th base of SEQ ID NO: 27 is A while the 17627th base of SEQ ID NO: 28 corresponding to said position is C.

For detecting this change, fragments of 126 bp are first amplified by PCR from a region surrounding said position using the following primer pair:

| | |
|---|---|
| P9493 BslI F: | |
| 5′-gcgatcttatacgcatactatgcg-3′ | (SEQ ID NO:41) |

(corresponding to bases 6129-6152 of SEQ ID NO:27)
(corresponding to bases 17529-17552 of SEQ ID
NO:28)
and

| | |
|---|---|
| P9493 BslI R: | |
| 5′-aaagtctttgttccttcaccaagg-3′ | (SEQ ID NO:42) |

(corresponding to bases 6254-6231 of SEQ ID NO:27)
(corresponding to bases 17654-17631 of SEQ ID
NO:28).

The amplification products can be visualized by electrophoresis on an agarose gel after treatment with BslI. Thus, the change can be detected as a difference in mobility in the agarose gel due to the difference in the length of DNA after BslI treatment because the amplification products from Asominori DNA having a BslI recognition sequence (CCNNNNNNNGG) are cleaved with BslI while the amplification products from IR24 DNA are not cleaved with BslI for the lack of the BslI recognition sequence.

This marker was developed by applying the dCAPS method (Michaels and Amasino 1998, Neff et al., 1998). Specifically, g is substituted for a at the base 6236 of SEQ ID NO: 27 and the base 17636 of SEQ ID NO: 28 by the use of P9493 BslI R primer described above. Thus, the fragments from Asominori DNA come to have a sequence of CCtttccttG at 17626-17636 of SEQ ID NO: 28 so that they are cleaved with BslI.

(3) Development of PCR Marker P23945 MboI

Comparison between the genomic base sequence corresponding to the IR24 contig (SEQ ID NO: 27) and the genomic base sequence corresponding to the Asominori contig (SEQ ID NO: 28) determined in Example 2 revealed that the 20680th base of SEQ ID NO: 27 is G while the 32079th base of SEQ ID NO: 28 corresponding to said position is A.

For detecting this change, fragments of 260 bp are first amplified by PCR from a region surrounding said position using the following primer pair:

| | |
|---|---|
| P23945 MboI F: | |
| 5′-gaggatttatcaaaacaggatggacg-3′ | (SEQ ID NO:43) |

(corresponding to bases 20519-20544 of SEQ ID
NO:27)
(corresponding to bases 31918-31943 of SEQ ID
NO:28)
and

| | |
|---|---|
| P23945 MboI R: | |
| 5′-tgggcggcagcagtggaggataga-3′ | (SEQ ID NO:44) |

(corresponding to bases 20778-20755 of SEQ ID
NO:27)
(corresponding to bases 32177-32154 of SEQ ID
NO:28).

The amplification products can be visualized by electrophoresis on an agarose gel after treatment with MboI. Thus, the change can be detected as a difference in mobility in the agarose gel due to the difference in the length of DNA after MboI treatment because the amplification products from IR24 DNA having an MboI recognition sequence (GATC) are cleaved with MboI while the amplification products from Asominori DNA are not cleaved with MboI for the lack of the MboI recognition sequence.
(4) Development of PCR Marker P41030 TaqI

Comparison between the genomic base sequence corresponding to the IR24 contig (SEQ ID NO: 27) and the genomic base sequence corresponding to the Asominori contig (SEQ ID NO: 28) determined in Example 2 revealed that the 45461st base of SEQ ID NO: 27 is A while the 49164th base of SEQ ID NO: 28 corresponding to said position is G.

For detecting this change, fragments of 280 bp are first amplified by PCR from a region surrounding said position using the following primer pair:

| | |
|---|---|
| P41030 TaqI F: | |
| 5′-aagaagggagggttatagaatctg-3′ | (SEQ ID NO:45) |

(corresponding to bases 45369-45392 of SEQ ID
NO:27)
(corresponding to bases 49072-49095 of SEQ ID
NO:28)
and

| | |
|---|---|
| P41030 TaqI R: | |
| 5′-atatcaggactaacaccactgctc-3′ | (SEQ ID NO:46) |

(corresponding to bases 45648-45625 of SEQ ID
NO:27)
(corresponding to bases 49351-49328 of SEQ ID
NO:28).

The amplification products can be visualized by electrophoresis on an agarose gel after treatment with TaqI. Thus, the change can be detected as a difference in mobility in the agarose gel due to the difference in the length of DNA after TaqI treatment because the amplification products from Asominori DNA having a TaqI recognition sequence (TCGA) are cleaved with TaqI while the amplification products from IR24 DNA are not cleaved with TaqI for the lack of the TaqI recognition sequence.
(5) Development of PCR Marker P45177 BstUI

Comparison between the genomic base sequence corresponding to the IR24 contig (SEQ ID NO: 27) and the genomic base sequence corresponding to the Asominori contig (SEQ ID NO: 28) determined in Example 2 revealed that the 49609th base of SEQ ID NO: 27 is A while the 53311st base of SEQ ID NO: 28 corresponding to said position is G.

For detecting this change, fragments of 812 bp are first amplified by PCR from a region surrounding said position using the following primer pair:

| | |
|---|---|
| P45177 BstUI F: | |
| 5′-acgagtagtagcgatcttccagcg-3′ | (SEQ ID NO:47) |

(corresponding to bases 49355-49378 of SEQ ID
NO:27)
(corresponding to bases 53057-53080 of SEQ ID
NO:28)
and
P45177 BstUI R:

| | |
|---|---|
| 5′-cagcgtgaaactaaaaacggaggc-3′ | (SEQ ID NO:48) |

(corresponding to bases 50166-50143 of SEQ ID
NO:27)
(corresponding to bases 53868-53845 of SEQ ID
NO:28).

The amplification products can be visualized by electrophoresis on an agarose gel after treatment with BstUI. Thus, the change can be detected as a difference in mobility in the agarose gel due to the difference in the length of DNA after BstUI treatment because the amplification products from IR24 DNA having a BstUI recognition sequence (CGCG) at two positions are cleaved into 3 fragments with BstUI while the amplification products from Asominori DNA having the BstUI recognition sequence at three positions are cleaved with BstUI into four fragments.
(6) Development of PCR Marker B60304 MspI

Comparison between the genomic base sequence corresponding to the IR24 contig (SEQ ID NO: 27) determined in Example 2 and the base sequence of the BAC clone described above (Accession No. AC068923) revealed that the 56368th base of SEQ ID NO: 27 is T while the base of AC068923 corresponding to said position is C.

For detecting this change, fragments of about 330 bp are first amplified by PCR from a region surrounding said position using the following primer pair:

| | |
|---|---|
| B60304 MspI F: | |
| 5′-atcccacatcatcataatccgacc-3′ | (SEQ ID NO:49) |

(corresponding to bases 56149-56172 of SEQ ID
NO:27)
and

| | |
|---|---|
| B60304 MspI R: | |
| 5′-agcttctcccttggatacggtggcg-3′ | (SEQ ID NO:50) |

(corresponding to bases 56479-56455 of SEQ ID
NO:27).

The amplification products can be visualized by electrophoresis on an agarose gel after treatment with MspI. Thus, the change can be detected as a difference in mobility in the agarose gel due to the difference in the length of DNA after MspI treatment because the amplification products from Nipponbare DNA having an MspI recognition sequence (CCGG) are cleaved with MspI while the amplification products from IR24 DNA are not cleaved with MspI for the lack of the MspI recognition sequence.

This marker was developed by applying the dCAPS method. Specifically, t is substituted for g at base 56463 of SEQ ID NO: 27 by the use of B60304 MspI R primer. As a result, the MspI recognition sequence of bases 56460-56463 of SEQ ID NO: 27 changes from CCGG into ccgt so that the fragments from SEQ ID NO: 27 become unable to be cleaved with MspI. Thus, the fragments from IR24 have no MspI recognition sequence, while DNA from Nipponbare has the MspI recognition sequence at one position in a region corresponding to bases 56367-56370 of SEQ ID NO: 27.

(7) Development of PCR Marker B59066 BsaJI

Comparison between the genomic base sequence corresponding to the IR24 contig (SEQ ID NO: 27) determined in Example 2 and the base sequence of the BAC clone described above (Accession No. AC068923) revealed that the 57629th base of SEQ ID NO: 27 is C while the base of AC068923 corresponding to said position is CC.

For detecting this change, fragments of about 420 bp are first amplified by PCR from a region surrounding said position using the following primer pair:

| | |
|---|---|
| B59066 BsaJI F: | |
| 5′-atttgttggttagttgcggctgag-3′ | (SEQ ID NO:51) |

(corresponding to bases 57563-57586 of SEQ ID
NO:27)
and

| | |
|---|---|
| B59066 BsaJI R: | |
| 5′-gcccaaactcaaaaggagagaacc-3′ | (SEQ ID NO:52) |

(corresponding to bases 57983-57960 of SEQ ID
NO:27).

The amplification products can be visualized by electrophoresis on an agarose gel after treatment with BsaJI. Thus, the change can be detected as a difference in mobility in the agarose gel due to the difference in the length of DNA after BsaJI treatment because the amplification products from Nipponbare DNA having a BsaJI recognition sequence (CCNNGG) are cleaved with BsaJI while the amplification products from IR24 DNA are not cleaved with BsaJI for the lack of the BsaJI recognition sequence.
(8) Development of PCR Marker B56691 XbaI

Comparison between the genomic base sequence corresponding to the IR24 contig (SEQ ID NO: 27) determined in Example 2 and the base sequence of the BAC clone described above (Accession No. AC068923) revealed that the 66267th base of SEQ ID NO: 27 is G while the base of AC068923 corresponding to said position is C.

For detecting this change, fragments of about 670 bp are first amplified by PCR from a region surrounding said position using the following primer pair:

| | |
|---|---|
| B56691 XbaI F: | |
| 5′-cctcaagtctcccctaaagccact-3′ | (SEQ ID NO:53) |

(corresponding to bases 66129-66152 of SEQ ID
NO:27)
and

| | |
|---|---|
| B56691 XbaI R: | |
| 5′-gctctactgctgataaaccgtgag-3′ | (SEQ ID NO:54) |

(corresponding to bases 66799-66776 of SEQ ID
NO:27).

The amplification products can be visualized by electrophoresis on an agarose gel after treatment with XbaI. Thus, the change can be detected as a difference in mobility in the agarose gel due to the difference in the length of DNA after XbaI treatment because the amplification products from Nipponbare DNA having an XbaI recognition sequence (TCTAGA) are cleaved with XbaI while the amplification products from IR24 DNA are not cleaved with XbaI for the lack of the XbaI recognition sequence.
(9) Development of PCR Marker B53627 BstZ17I

Comparison between the genomic base sequence corresponding to the IR24 contig (SEQ ID NO: 27) determined in Example 2 and the base sequence of the BAC clone described above (Accession No. AC068923) revealed that the 69331st base of SEQ ID NO: 27 is T while the base of AC068923 corresponding to said position is C.

For detecting this change, fragments of about 620 bp are first amplified by PCR from a region surrounding said position using the following primer pair:

| | |
|---|---|
| B53627 BstZ17I F: | |
| 5′-tggatggactatgtggggtcagtc-3′ | (SEQ ID NO:55) |

(corresponding to bases 68965-68988 of SEQ ID
NO:27)
and

| | |
|---|---|
| B53627 BstZ17I R: | |
| 5′-agtggaagtggagagagtagggag-3′ | (SEQ ID NO:56) |

(corresponding to bases 69582-69559 of SEQ ID
NO:27).

The amplification products can be visualized by electrophoresis on an agarose gel after treatment with BstZ17I. Thus, the change can be detected as a difference in mobility in the agarose gel due to the difference in the length of DNA after BstZ17I treatment because the amplification products from IR24 DNA having a BstZ17I recognition sequence (GTATAC) are cleaved with BstZ17I while the amplification products from Nipponbare DNA are not cleaved with BstZ17I for the lack of the BstZ17I recognition sequence.
(10) Development of PCR Marker B40936 MseI

Development of all the following PCR markers (10)-(12) relates to a study of the base sequences corresponding to further downstream regions (3′) of base 76363 at the 3′end of SEQ ID NO: 27.

The following primer pair was designed for the base sequence of the BAC clone described above (Accession No. AC068923):

| | |
|---|---|
| 5′-tacgacgccatttcactccattgc-3′ | (SEQ ID NO:57) |
| and | |
| 5′-catttctctatgggcgttgctctg-3′. | (SEQ ID NO:58) |

PCR was routinely performed using this primer pair in combination with total DNAs of MS-FR Koshihikari (genotype of the Rf-1 locus: Rf-1 Rf-1) and Koshihikari as templates. The resulting amplification products of about 1300 bp were electrophoresed on an agarose gel and then purified by QIAEXII (QIAGEN). Analysis of the base sequence of the purified DNA by a DNA sequencer 377 (ABI) showed several polymorphisms.

One of them can be detected by PCR amplification of a region surrounding said position using the following primer pair:

| | |
|---|---|
| B40936 MseI F: | |
| 5′-acctgtaggtatggcaccttcaacac-3′ | (SEQ ID NO:59) |
| and | |
| B40936 MseI R: | |
| 5′-ccaaggaacgaagttcaaatgtatgg-3′. | (SEQ ID NO:60) |

The amplification products can be visualized by electrophoresis on an agarose gel after treatment with MseI. Thus, the change can be detected as a difference in mobility in the agarose gel due to the difference in the length of DNA after MseI treatment because the amplification products from MS-FR Koshihikari (Rf-1 Rf-1) DNA having an MseI recognition sequence (TTAA) are cleaved with MseI while the amplification products from Koshihikari DNA are not cleaved with MseI for the lack of the MseI recognition sequence.

This marker was developed by applying the dCAPS method.

(11) Development of PCR Marker B19839 MwoI

The following primer pair was designed for the base sequence of the BAC clone described above (Accession No. AC068923):

| | |
|---|---|
| 5′-tgatgtgtttgggcatccctttcg-3′ | (SEQ ID NO:61) |
| and | |
| 5′-gagataggggacgacagacacgac-3′. | (SEQ ID NO:62) |

PCR was routinely performed using this primer pair in combination with total DNAs of MS-FR Koshihikari (genotype of the Rf-1 locus: Rf-1 Rf-1) and Koshihikari as templates. The resulting amplification products of about 1200 bp were electrophoresed on an agarose gel and then purified by QIAEXII (QIAGEN). Analysis of the base sequence of the purified DNA by a DNA sequencer 377 (ABI) showed several polymorphisms.

One of them can be detected by PCR amplification of a region surrounding said position using the following primer pair:

| | |
|---|---|
| B19839 MwoI F: | |
| 5′-tcctatggctgtttagaaactgcaca-3′ | (SEQ ID NO:63) |
| and | |
| B19839 MwoI R: | |
| 5′-caagttcaaacataactggcgttg-3′. | (SEQ ID NO:64) |

The amplification products can be visualized by electrophoresis on an agarose gel after treatment with MwoI. Thus, the change can be detected as a difference in mobility in the agarose gel due to the difference in the length of DNA after MwoI treatment because the amplification products from Koshihikari DNA having an MwoI recognition sequence (GCNNNNNNNGC) are cleaved with MwoI while the amplification products from MS-FR Koshihikari (Rf-1 Rf-1) DNA are not cleaved with MwoI for the lack of the MwoI recognition sequence.

This marker was developed by applying the dCAPS method.

(12) Development of PCR Marker B2387 BfaI

The following primer pair was designed for the base sequence of the BAC clone described above (Accession No. AC068923):

| | |
|---|---|
| 5′-cactgtcctgtaagtgtgctgtgc-3′ | (SEQ ID NO:65) |
| and | |
| 5′-caagcgtgtgataaaatgtgacgc-3′. | (SEQ ID NO:66) |

PCR was routinely performed using this primer pair in combination with total DNAs of MS-FR Koshihikari (genotype of the Rf-1 locus: Rf-1 Rf-1) and Koshihikari as templates. The resulting amplification products of about 1300 bp were electrophoresed on an agarose gel and then purified by QIAEXII (QIAGEN). Analysis of the base sequence of the purified DNA by a DNA sequencer 377 (ABI) showed several polymorphisms.

One of them can be detected by PCR amplification of a region surrounding said position using the following primer pair:

| | |
|---|---|
| B2387 BfaI F: | |
| 5′-tgcctactgccattactatgtgac-3′ | (SEQ ID NO:67) |
| and | |
| B2387 BfaI R: | |
| 5′-acatactaccgtaaatggtctctg-3′. | (SEQ ID NO:68) |

The amplification products can be visualized by electrophoresis on an agarose gel after treatment with BfaI. Thus, the change can be detected as a difference in mobility in the agarose gel due to the difference in the length of DNA after BfaI treatment because the amplification products from Koshihikari DNA having an BfaI recognition sequence (CTAG) are cleaved with BfaI while the amplification products from MS-FR Koshihikari (Rf-1 Rf-1) DNA are not cleaved with BfaI for the lack of the BfaI recognition sequence.
(13) Segregation Analysis

Two recombinants between the Rf-1 and S12564 Tsp509I loci (RS1 and RS2) and 8 recombinants between the Rf-1 and C1361 MwoI loci (RC1 to RC8) obtained in Example 1 were genotyped at the 12 DNA marker loci developed in (1) to (12) above. The results are shown in Table 4 along with the genotypes of each recombinant at the S12564 Tsp509I and C1361 MwoI loci.

TABLE 4
Genotypes of recombinants proximal to the Rf-1 locus at various marker loci

| | Locus | | | | | | | | | |
|---|---|---|---|---|---|---|---|---|---|---|
| | RS1 | RS2 | RC1 | RC2 | RC3 | RC4 | RC5 | RC6 | RC7 | RC8 |
| S12564 Tsp509I | J | J | H | H | H | H | H | H | H | H |
| P4497 MboI | J | J | H | H | H | H | H | H | H | H |
| P9493 BsiI | H | H | H | H | H | H | H | H | H | H |
| P23945 MboI | H | H | H | H | H | H | H | H | H | H |
| P41030 TaqI | H | H | H | H | H | H | H | H | H | H |
| P45177 BstUI | H | H | H | H | H | H | H | H | H | H |
| B60304 MspI | H | H | H | H | H | H | H | H | H | H |
| B59066 BsaJI | H | H | H | H | H | H | H | H | H | H |
| B56691 XbaI | H | H | H | H | H | H | H | J | H | H |
| B53627 BstZ17I | H | H | H | H | H | H | H | J | H | H |
| B40936 MseI | H | H | H | H | H | H | H | J | H | H |
| B19839 MwoI | H | H | H | H | H | J | H | J | H | H |
| B2387 BfaI | H | H | H | H | H | J | H | J | H | J |
| C1361 MwoI | H | H | J | J | J | J | J | J | J | J |

J: Homozygous for Koshihikari
H: Heterozygous for Koshihikari/MS-FR Koshihikari

Table 4 shows that all the recombinants have an indica-derived Rf-1 chromosomal region between P9493 BslI and 59066 BsaJI. This result showed that recombinant pollens having the chromosomal organization as shown in FIG. 3 have pollen fertility, i.e. the Rf-1 gene is functional in these pollens. This means that a sequence determining the presence of the function of the Rf-1 gene is included in the indica region common to these recombinant pollens, i.e. in a region from the P4497 MboI to B56691 XbaI loci (about 65 kb) as estimated at maximum.

However, there is a possibility that it is important for the expression of the genetic function of the Rf-1 gene that the Rf-1 gene is partially of the indica genotype, and that the genetic function may not be significantly changed whether the remaining regions are of the japonica or indica genotype. Therefore, it cannot be concluded that the common indica region above (bases 1239-66267 of SEQ ID NO: 27) completely contains the entire Rf-1 gene. However, it is thought that at least SEQ ID NO: 27 completely contains the entire Rf-1 gene for the following reasons:

1) the size of a gene is normally several kilobases, and rarely exceeds 10 kb;

2) the genomic base sequence of IR24 determined by the present invention (SEQ ID NO: 27) completely contains the common indica region above;

3) the 5′ end of SEQ ID NO: 27 is located 1238 bp upstream of the 5′ end of the common indica region above and forms a part of another gene (S12564); and

4) the 3′ end of SEQ ID NO: 27 is located 10096 bp downstream of the 3′ end of the common indica region above.

### Example 4

### Complementation Assay for a 9.7 kb Fragment from XSE1

(Materials and Methods)

The λ phage clone XSE1 (FIGS. 1 and 5) was completely digested with NotI and electrophoresed on an agarose gel. The separated 9.7 kb fragment (including bases 1-9657 of SEQ ID NO: 27) was purified by QIAEXII (QIAGEN).

On the other hand, an intermediate vector pSB200 having a hygromycin-resistant gene cassette was prepared on the basis of pSB11 (Komari et al., supra.). Specifically, a nopaline synthase terminator (Tnos) was first fused to a ubiquitin promoter and a ubiquitin intron (Pubi-ubiI). A hygromycin-resistant gene (HYG(R)) was inserted between ubiI and Tnos of the resulting Pubi-ubiI-Tnos complex to give an assembly of Pubi-ubiI-HYG(R)-Tnos. This assembly was fused to a HindIII/EcoRI fragment of pSB11 to give pKY205. Linker sites for adding restriction enzyme sites NotI, NspV, EcoRV, KpnI, SacI, EcoRI were inserted into the Hind III site upstream of Pubi of this pKY205 to give pSB200 having a hygromycin-resistant gene cassette.

After the plasmid vector pSB200 was completely digested with NotI, DNA was recovered by ethanol precipitation. The recovered DNA was dissolved in TE solution and then dephosphorylated by CIAP (TAKARA). The reaction solution was electrophoresed on an agarose gel, and then a vector fragment was purified from the gel using QIAEXII (QIAGEN).

The two fragments prepared above, i.e. a 9.7 kb fragment from XSE1 and a vector fragment were subjected to a ligation reaction using DNA Ligation Kit Ver. 1 (TAKARA). After the reaction, DNA was recovered by ethanol precipitation. The recovered DNA was dissolved in pure water (prepared by a Millipore system) and then mixed with E. coli DH5a cells, and the mixture was electroporated. After electroporation, the solution was cultured with shaking in LB medium (37° C., 1 hr) and then plated on an LB plate containing spectinomycin and warmed (37° C., 16 hr). Plasmids were isolated from 24 of the resulting colonies. Their restriction enzyme fragment length patterns and boundary base sequences were analyzed to select desired E. coli cells transformed with recombinant plasmids.

The E. coli cells selected above were used for triparental mating with the Agrobacterium tumefaciens strain LBA4404/pSB1 (Komari et al., 1996) and the helper E. coli strain HB101/pRK2013 (Ditta et al., 1980) according to the method of Ditta et al. (1980). Plasmids were isolated from 6 of the colonies formed on an AB plate containing spectinomycin and their restriction enzyme fragment length patterns were analyzed to select desired Agrobacterium cells.

The Agrobacterium cells selected above were used to transform MS Koshihikari (having BT cytoplasm and a nucleus gene substantially identical to Koshihikari) according to the method of Hiei et al. (1994). Necessary immature seeds of MS Koshihikari for transformation can be prepared by pollinating MS Koshihikari with Koshihikari.

Transformed plants were transferred to a greenhouse under long-day conditions after acclimation. 48 individuals grown to a stage suitable for transplantation were transplanted into 1/5000a Wagner pots (4 individuals/pot), and transferred into a greenhouse under short-day conditions 3-4 weeks after transplantation. About one month after heading, seed fertility was tested on standing plants.

(Results and Discussion)

All of the 48 transformed individuals were sterile. This indicates that the 9.7 kb insert fragment does not contain at least the full-length Rf-1 gene.

### Example 5

### Complementation Assay for a 14.7 kb Fragment from XSE7

(Materials and Methods)

The λ phage clone XSE7 (FIGS. 1 and 5) was completely digested with EcoRI and then DNA was recovered by ethanol precipitation. The recovered DNA was dissolved in TE solution and then blunted by DNA Blunting Kit (TAKARA). The reaction solution was electrophoresed on an agarose gel to separate a 14.7 kb fragment (including bases 2618-17261 of SEQ ID NO: 27), which was purified by QIAEXII (QIAGEN).

On the other hand, the plasmid vector pSB200 was completely digested with SacI and then DNA was recovered by ethanol precipitation. The recovered DNA was dissolved in TE solution and then dephosphorylated by CIAP (TAKARA) and DNA was recovered by ethanol precipitation. The recovered DNA was dissolved in TE solution and then blunted by DNA Blunting Kit (TAKARA). The reaction solution was electrophoresed on an agarose gel, and then a vector fragment was purified from the gel using QIAEXII (QIAGEN).

The two fragments prepared above, i.e. the 14.7 kb fragment from XSE7 and the vector fragment were subjected to a ligation reaction using DNA Ligation Kit Ver. 1 (TAKARA). Subsequently, transformed plants were prepared and studied according to the method described in Example 4.

(Results and Discussion)

All of the 48 transformed individuals were sterile. This indicates that the 14.7 kb insert fragment does not contain at least the full-length Rf-1 gene.

### Example 6

### Complementation Assay for a 21.3 kb Fragment from XSF4

(Materials and Methods)

The λ phage clone XSF4 (FIGS. 1 and 5) was partially digested with NotI and electrophoresed on an agarose gel. The separated 21.3 kb fragment (including bases 12478-33750 of SEQ ID NO: 27) was purified by QIAEXII (QIAGEN).

On the other hand, the plasmid vector pSB200 was completely digested with NotI and then DNA was recovered by ethanol precipitation. The recovered DNA was dissolved in TE solution and then dephosphorylated by CIAP (TAKARA). The reaction solution was electrophoresed on an agarose gel, and then a vector fragment was purified from the gel using QIAEXII (QIAGEN).

The two fragments prepared above, i.e. the 21.3 kb fragment from XSF4 and the vector fragment were subjected to a ligation reaction using DNA Ligation Kit Ver. 1 (TAKARA). Subsequently, transformed plants were prepared and studied according to the method described in Example 4.

(Results and Discussion)

All of the 48 transformed individuals were sterile. This indicates that the 21.3 kb insert fragment does not contain at least the full-length Rf-1 gene.

### Example 7

### Complementation Assay for a 13.2 kb Fragment from XSF20

(Materials and Methods)

The λ phage clone XSF20 (FIGS. 1 and 5) was completely digested with SalI and then DNA was recovered by ethanol precipitation. The recovered DNA was dissolved in TE solution and then blunted by DNA Blunting Kit (TAKARA). The reaction solution was electrophoresed on an agarose gel to separate a 13.2 kb fragment (including bases 26809-40055 of SEQ ID NO: 27), which was purified by QIAEXII (QIAGEN).

On the other hand, the plasmid vector pSB200 was completely digested with EcoRV and then DNA was recovered by ethanol precipitation. The recovered DNA was dissolved in TE solution and then dephosphorylated by CIAP (TAKARA). The reaction solution was electrophoresed on an agarose gel, and then a vector fragment was purified from the gel using QIAEXII (QIAGEN).

The two fragments prepared above, i.e. the 13.2 kb fragment from XSF20 and the vector fragment were subjected to a ligation reaction using DNA Ligation Kit Ver. 1 (TAKARA). Subsequently, transformed plants were prepared and studied according to the method described in Example 4.

(Results and Discussion)

All of the 44 transformed individuals were sterile. This indicates that the 13.2 kb insert fragment does not contain at least the full-length Rf-1 gene.

### Example 8

### Complementation Assay for a 16.2 kb Fragment from XSF18

(Materials and Methods)

The λ phage clone XSF18 is identical to XSF20 at the 5′ and 3′ ends (bases 20328 and 41921 of SEQ ID NO: 27, respectively), but lacks internal bases 33947-38591. Thus, it comprises bases 20328-33946 and 38592-41921 of SEQ ID NO: 27. This is because clone XSF18 was initially isolated but found to contain the above deletion during amplification after isolation, and therefore, the amplification step was freshly taken to isolate a complete clone designated XSF20.

The λ phage clone XSF18 (FIG. 5) was completely digested with NotI and electrophoresed on an agarose gel. The separated 16.2 kb fragment (including bases 21065-33946 and 38592-41921 of SEQ ID NO: 27) was purified by QIAEXII (QIAGEN).

On the other hand, the plasmid vector pSB200 was completely digested with NotI and then DNA was recovered by ethanol precipitation. The recovered DNA was dissolved in TE solution and then dephosphorylated by CIAP (TAKARA). The reaction solution was electrophoresed on an agarose gel, and then a vector fragment was purified from the gel using QIAEXII (QIAGEN).

The two fragments prepared above, i.e. the 16.2 kb fragment from XSF18 and the vector fragment were subjected to a ligation reaction using DNA Ligation Kit Ver. 1 (TAKARA). Subsequently, transformed plants were prepared and studied according to the method described in Example 4.

(Results and Discussion)

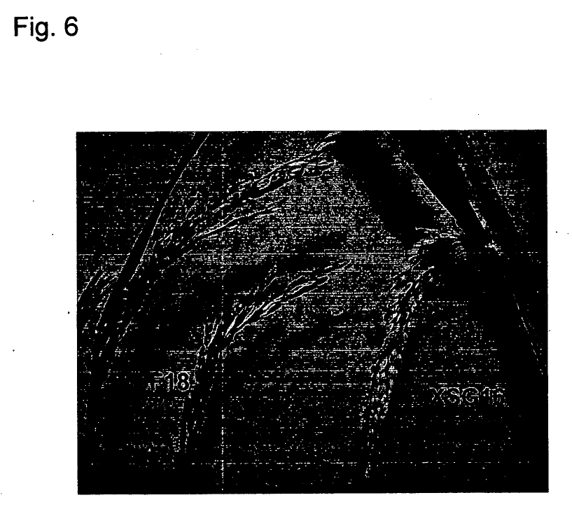

All of the 48 transformed individuals were sterile (FIG. 6). This indicates that the 16.2 kb insert fragment does not contain at least the full-length Rf-1 gene.

### Example 9

### Complementation Assay for a 12.6 kb Fragment from XSG22

(Materials and Methods)

The λ phage clone XSG22 (FIGS. 1 and 5) was partially digested with NotI and electrophoresed on an agarose gel. The separated 12.6 kb fragment (including bases 31684-44109 of SEQ ID NO: 27) was purified by QIAEXII (QIAGEN).

On the other hand, the plasmid vector pSB200 was completely digested with NotI and then DNA was recovered by ethanol precipitation. The recovered DNA was dissolved in TE solution and then dephosphorylated by CIAP (TAKARA). The reaction solution was electrophoresed on an agarose gel, and then a vector fragment was purified from the gel using QIAEXII (QIAGEN).

The two fragments prepared above, i.e. the 12.6 kb fragment from XSG22 and the vector fragment were subjected to a ligation reaction using DNA Ligation Kit Ver. 1 (TAKARA). Subsequently, transformed plants were prepared and studied according to the method described in Example 4.

(Results and Discussion)

All of the 48 transformed individuals were sterile. This indicates that the 12.6 kb insert fragment does not contain at least the full-length Rf-1 gene.

### Example 10

### (1) Complementation Assay for a 15.7 kb Fragment from XSG16

(Materials and Methods)

The λ phage clone XSG16 (FIGS. 1 and 5) was partially digested with NotI and electrophoresed on an agarose gel. The separated 15.7 kb fragment (including bases 38538-54123 of SEQ ID NO: 27) was purified by QIAEXII (QIAGEN).

On the other hand, the plasmid vector pSB200 was completely digested with NotI and then DNA was recovered by ethanol precipitation. The recovered DNA was dissolved in TE solution and then dephosphorylated by CIAP (TAKARA). The reaction solution was electrophoresed on an agarose gel, and then a vector fragment was purified from the gel using QIAEXII (QIAGEN).

The two fragments prepared above, i.e. the 15.7 kb fragment from XSG16 and the vector fragment were subjected to a ligation reaction using DNA Ligation Kit Ver. 1 (TAKARA). Subsequently, transformed plants were prepared and studied according to the method described in Example 4.

(Results and Discussion)

Of the 47 transformed individuals, at least 37 individuals clearly restored fertility (FIG. 6). This indicates that 15586 bases (bases 38538-54123 of SEQ ID NO: 27) derived from rice (IR24) in the 15.7 kb insert fragment include the full-length Rf-1 gene.

### (2) Complementation Assay for an Internal 11.4 kb Fragment in XSG16

(Materials and Methods)

The λ phage clone XSG16 was completely digested with AlwNI and BsiWI and then DNA was recovered by ethanol precipitation. The recovered DNA was dissolved in TE solution and then blunted by DNA Blunting Kit (TAKARA). The reaction solution was electrophoresed on an agarose gel to separate a 11.4 kb fragment, which was purified by QIAEXII (QIAGEN).

The plasmid vector pSB11 (Komari et al. Plant Journal, 1996) was completely digested with SmaI and then DNA was recovered by ethanol precipitation. The recovered DNA was dissolved in TE solution and then dephosphorylated by CIAP (TAKARA). The reaction solution was electrophoresed on an agarose gel, and then a vector fragment was purified from the gel using QIAEXII (QIAGEN).

The two fragments prepared above were subjected to a ligation reaction using DNA Ligation Kit Ver. 1 (TAKARA). After the reaction, DNA was recovered by ethanol precipitation. The recovered DNA was dissolved in pure water (prepared by a Millipore system) and then mixed with E. coli DH5a cells, and the mixture was electroporated. After electroporation, the solution was cultured with shaking in LB medium (37° C., 1 hr) and then plated on an LB plate containing spectinomycin and warmed (37° C., 16 hr). Plasmids were isolated from 14 of the resulting colonies, and their restriction enzyme fragment length patterns and boundary base sequences were analyzed to select desired E. coli cells.

The E. coli cells selected above were used for triparental mating with the Agrobacterium tumefaciens strain LBA4404/pSB4U (Takakura et al., Japanese Patent Application No. 2001-269982 (WO02/019803 A1)) and the helper E. coli strain HB101/pRK2013 (Ditta et al., 1980) according to the method of Ditta et al. (1980). Plasmids were isolated from 12 of the colonies formed on an AB plate containing spectinomycin and their restriction enzyme fragment length patterns were analyzed to select desired Agrobacterium cells.

The Agrobacterium cells selected above were used to transform MS Koshihikari (having BT cytoplasm and a nucleus gene substantially identical to Koshihikari) according to the method of Hiei et al. (1994). Necessary immature seeds of MS Koshihikari for transformation can be prepared by pollinating MS Koshihikari with Koshihikari.

Transformed plants were transferred to a greenhouse under long-day conditions after acclimation. 120 individuals grown to a stage suitable for transplantation were transplanted into 1/5000a Wagner pots (4 individuals/pot), and transferred into a greenhouse under short-day conditions about one month after transplantation. About one month after heading, one typical ear was sampled from each plant to evaluate seed fertility (the percentage of fertile paddies to total paddies).

(Results and Discussion)

Of the 120 transformed individuals, 59 individuals showed seed fertility of 10% or more, among which 19 individuals showed seed fertility of 70% or more. This indicates that the 11.4 kb insert fragment (bases 42357-53743 of SEQ ID NO: 27) contains an essential Rf-1 gene region for expressing a fertility restoring function.

### (3) Complementation Assay for an Internal 6.8 kb Fragment in XSG16

(Materials and Methods)

The λ phage clone XSG16 was completely digested with HpaI and AlwNI and electrophoresed on an agarose gel. The separated 6.8 kb fragment was purified by QIAEXII (QIAGEN).

The subsequent procedures including the preparation of the plasmid vector pSB11 were performed according to the method in (2) above.

(Results and Discussion)

Of the 120 transformed individuals, 67 individuals showed seed fertility of 10% or more, among which 26 individuals showed seed fertility of 70% or more. This indicates that the 6.8 kb insert fragment (bases 42132-48883 of SEQ ID NO: 27) contains an essential Rf-1 gene region for expressing a fertility restoring function.

### Example 11

### Complementation Assay for a 16.9 kb Fragment from XSG8

(Materials and Methods)

The λ phage clone XSG8 (FIGS. 1 and 5) was completely digested with NotI and electrophoresed on an agarose gel. The separated 16.9 kb fragment (including bases 46558-63364 of SEQ ID NO: 27) was purified by QIAEXII (QIAGEN).

On the other hand, the plasmid vector pSB200 was completely digested with NotI and then DNA was recovered by ethanol precipitation. The recovered DNA was dissolved in TE solution and then dephosphorylated by CIAP (TAKARA). The reaction solution was electrophoresed on an agarose gel, and then a vector fragment was purified from the gel using QIAEXII (QIAGEN).

The two fragments prepared above, i.e. the 16.9 kb fragment from XSG8 and the vector fragment were subjected to a ligation reaction using DNA Ligation Kit Ver. 1 (TAKARA). Subsequently, transformed individuals were prepared and studied according to the method described in Example 4.

(Results and Discussion)

All of the 48 transformed individuals were sterile. This indicates that the 16.9 kb insert fragment does not contain at least the full-length Rf-1 gene.

### Example 12

### Complementation Assay for a 20.0 kb Fragment from XSH18

(Materials and Methods)

The λ phage clone XSH18 (FIGS. 1 and 5) was completely digested with NotI and electrophoresed on an agarose gel. The separated 20.0 kb fragment (including bases 56409-76363 of SEQ ID NO: 27) was purified by QIAEXII (QIAGEN).

On the other hand, the plasmid vector pSB200 was completely digested with NotI and then DNA was recovered by ethanol precipitation. The recovered DNA was dissolved in TE solution and then dephosphorylated by CIAP (TAKARA). The reaction solution was electrophoresed on an agarose gel, and then a vector fragment was purified from the gel using QIAEXII (QIAGEN).

The two fragments prepared above, i.e. the 20.0 kb fragment from XSH18 and the vector fragment were subjected to a ligation reaction using DNA Ligation Kit Ver. 1 (TAKARA). Subsequently, transformed individuals were prepared and studied according to the method described in Example 4.

(Results and Discussion)

All of the 44 transformed individuals were sterile. This indicates that the 20.0 kb insert fragment does not contain at least the full-length Rf-1 gene.

### Example 13

### Complementation Assay for a 19.7 kb Fragment from an Overlapping Region of XSG8 and XSH18

(Materials and Methods)

A plasmid (XSG8SB200F) isolated from desired E. coli cells obtained by ligation in Example 11 was completely digested with SalI and StuI and electrophoresed on an agarose gel. The separated 12.8 kb fragment (including bases 50430-63197 of SEQ ID NO: 27) was purified by QIAEXII (QIAGEN).

On the other hand, a plasmid (XSH18SB200R) isolated from desired E. coli cells obtained by ligation in Example 12 was completely digested with SalI, StuI and XhoI and electrophoresed on an agarose gel to separate a 6.9 kb fragment (including bases 63194-70116 of SEQ ID NO: 27), which was purified by QIAEXII (QIAGEN).

Further, the plasmid vector pSB200 was completely digested with EcoRV and then DNA was recovered by ethanol precipitation. The recovered DNA was dissolved in TE solution and then dephosphorylated by CIAP (TAKARA). The reaction solution was electrophoresed on an agarose gel, and then a vector fragment was purified from the gel using QIAEXII (QIAGEN).

The three fragments prepared above, i.e. the 12.8 kb fragment from XSG8, the 6.9 kb fragment from XSH18 and the vector fragment were subjected to a ligation reaction using DNA Ligation Kit Ver. 1 (TAKARA). The ligation product contains a 19.7 kb fragment from an overlapping region of XSG8 and XSH18 (including 50430-70116 of SEQ ID NO: 27) (XSX1 in FIG. 5). Subsequently, transformed individuals were prepared and studied according to the method described in Example 4.

(Results and Discussion)

All of the 40 transformed individuals were sterile. This indicates that the 19.7 kb insert fragment does not contain at least the full-length Rf-1 gene.

1. A method for restoring rice fertility comprising introducing a nucleic acid into rice, wherein the nucleic acid has the base sequence of SEQ ID NO.27, or has a base sequence which is identical to at least 70% of the base sequence of SEQ ID NO.27, and which functions to restore fertility.

2. A method for restoring rice fertility comprising introducing a nucleic acid into rice, wherein the nucleic acid has the base sequence of bases 38538-54123 of SEQ ID NO.27, or has a base sequence which is identical to at least 70% of the base sequence of bases 38538-54123 of SEQ ID NO.27, and which functions to restore fertility.

3. A method for restoring rice fertility comprising introducing a nucleic acid into rice, wherein the nucleic acid has the base sequence of bases 42132-48883 of SEQ ID NO.27, or has a base sequence which is identical to at least 70% of the base sequence of bases 42132-48883 of SEQ ID NO.27, and which functions to restore fertility.

4. The method of any one of claims 1-3, wherein the nucleic acid having a base sequence which is identical to at least 70% of the base sequence of SEQ ID NO.27 or of the base sequence of bases 38538-54123 of SEQ ID NO.27, and which meets at least one of the following requirements 1) and 2):
1) a base corresponding to the base 45461 of SEQ ID NO.27 is A; and
2) a base corresponding to the base 49609 of SEQ ID NO.27 is A.

5. The method of claim 1, wherein the nucleic acid comprising the rice restorer gene locus introduced into rice, does not comprise any constitutional gene other than the rice restorer gene.

6. A method for discerning whether or not a subject rice individual or a seed thereof has the rice restorer gene (the Rf-1 gene) or not, wherein the method utilizing a fact that a sequence determining the presence of the function of the Rf-1 gene positions between the polymorphism detection marker loci P4497 MboI and B56691 Xba I on rice chromosome 10.

7. The method of claim 6 wherein the Rf-1 gene exists in a nucleic acid having the base sequence of SEQ ID NO.27, or a nucleic acid having a base sequence which is identical to at least 70% of the base sequence of SEQ ID NO.27.

8. The method of claim 6 wherein the Rf-1 gene exists in a nucleic acid having the base sequence of bases 38538-54123 of SEQ ID NO.27, or a nucleic acid having a base sequence which is identical to at least 70% of the base sequence of bases 38538-54123 of SEQ ID NO.27.

9. The method of claim 6 wherein the Rf-1 gene exists in a nucleic acid having the base sequence of bases 42132-48883 of SEQ ID NO.27, or a nucleic acid having a base sequence which is identical to at least 70% of the base sequence of bases 42132-48883 of SEQ ID NO.27.

10. The method of any one of claims 7-9 wherein the subject rice individual or the seed thereof is determined to have the Rf-1 gene, in the case that the nucleic acid having a base sequence which is identical to at least 70% of the base sequence of SEQ ID NO.27 or of the base sequence of bases 38538-54123 of SEQ ID NO.27, meets at least one of the following requirements 1) and 2):
1) a base corresponding to the base 45461 of SEQ ID NO.27 is A; and
2) a base corresponding to the base 49609 of SEQ ID NO.27 is A.

11. The method of claim 7, wherein the method comprises:
i) preparing a pair of primers based on a base sequence of adjacent regions including any one of the following base;
1) a base corresponding to the base 45461; or
2) a base corresponding to the base 49609;
to amplify both the base of the above and adjacent regions thereto;
ii) performing nucleic acid amplification reaction(s) using the genome DNA of the subject rice individual or the seed thereof as a template; and
iii) discerning the presence of the Rf-1 in the subject rice individual or the seed thereof based on polymorphism found in said nucleic acid amplification product.

12. The method of claim 11 wherein the subject rice individual or the seed thereof is determined to have the Rf-1 gene, in the case that the step iii) meets least one of the following requirements 1) and 2):
1) a region including a base corresponding to the base 45461 of SEQ ID NO.27 does not have any TaqI recognition sequence; and
2) a region including a base corresponding to the base 49609 of SEQ ID NO.27 does not have any BstU recognition sequence.

13. The method of claim 11 using a pair of primers having base sequences selected from the group consisting of SEQ ID NO:45 and 46, and SEQ ID NO:47 and 48.

14. A method for inhibiting the function of the Rf-1 gene to restore fertility by introducing an antisense having at least 100 continuous bases in length, and having a base sequence complementary to a nucleic acid having the base sequence of SEQ ID NO.27, or to a nucleic acid having a base sequence which is identical to at least 70% of the base sequence of SEQ ID NO.27, and which functions to restore fertility.

15. A method for inhibiting the function of the Rf-1 gene to restore fertility by introducing an antisense having at least 100 continuous bases in length, and being selected from base sequences complementary to a nucleic acid having the base sequence of bases 38538-54123 of SEQ ID NO.27, or to a nucleic acid having a base sequence which is identical to at least 70% of the base sequence of bases 38538-54123 of SEQ ID NO.27, and which functions to restore fertility.

16. A nucleic acid having the base sequence of SEQ ID NO.27, or a nucleic acid having a base sequence which is identical to at least 70% of the base sequence of SEQ ID NO.27, and which functions to restore fertility.

17. A nucleic acid having the base sequence of bases 38538-54123 of SEQ ID NO.27, or a nucleic acid having a base sequence which is identical to at least 70% of the base sequence of bases 38538-54123 of SEQ ID NO.27, and which functions to restore fertility.

18. A nucleic acid having the base sequence of bases 42132-48883 of SEQ ID NO.27, or a nucleic acid having a base sequence which is identical to at least 70% of the base sequence of bases 42132-48883 of SEQ ID NO.27, and which functions to restore fertility.
